(19) United States
(12) Patent Application Publication
(10) Pub. No.: US 2026/0193293 A1
(43) Pub. Date: Jul. 9, 2026

(54) PEPTIDES CAPABLE OF INHIBITING PROTEIN-PROTEIN INTERACTIONS AT GABAB1A SUBUNIT AND USES THEREOF

(71) Applicants: UNIVERSITY OF COPENHAGEN, Copenhagen K (DK); UNIVERSITÄT BASEL, Basel (CH)

(72) Inventors: Vita Sereikaite, Copenhagen K (DK); Ziyang Chen, Copenhagen K (DK); Pascal Rem, Basel (CH); Bernhard Bettler, Basel (CH); Kristian StrØmgaard, Copenhagen K (DK)

(21) Appl. No.: 19/132,926

(22) PCT Filed: Nov. 28, 2023
(86) PCT No.: PCT/EP2023/083377

§ 371 Date: May 27, 2025

(30) Foreign Application Priority Data
Nov. 28, 2022 (EP) 22209880.8

(51) Int. Cl. C07K 7/56; A61K 38/00; A61K 47/64; A61P 25/00; C07K 7/64

(52) U.S. Cl. CPC C07K 7/56; A61K 47/64; A61P 25/00; C07K 7/64; A61K 38/00; C07K 2319/10

(57) ABSTRACT

The present invention relates to peptides capable of inhibiting protein-protein interactions of GABA_{B}R1a receptors and uses thereof.

### TECHNICAL FIELD

The present invention relates to peptides capable of inhibiting protein-protein interactions at the GABA_{B1a} subunit of GABA_{B} receptors and uses thereof.

### BACKGROUND

Synaptic transmission is a process tightly regulated by release of neurotransmitters and it is dependent on a fine-tuned balance of neuronal excitation and inhibition. Neurotransmitters are released from a presynaptic neuron and bind to receptors on a postsynaptic neuron. Synaptic transmission at the excitatory synapses is mainly mediated and regulated by ionotropic and metabotropic glutamate receptors, respectively, which are activated by the excitatory neurotransmitter glutamate. In contrast, γ-amino butyric acid (GABA), the key inhibitory neurotransmitter in the central nervous system (CNS), activates ionotropic GABA_{A} and metabotropic GABA_{B} receptors (GBRs), which mediate and regulate inhibitory synaptic transmission, respectively. GBRs is involved in learning and memory, and their dysfunction has been implicated in neurological and psychiatric disorders such as epilepsy, spasticity, depression, anxiety, schizophrenia, Alzheimer's disease and cognitive deficits (Heaney et al. 2016). However, therapeutics targeting GBRs are very scarce as only two drugs, baclofen and γ-hydroxybutyrate (GHB), are currently used for the treatment of spasticity (Bowery et al. 2002), alcohol use disorders (Garbutt 2020) and narcolepsy (Wedin et al. 2006; Bay et al. 2014). Unfortunately, the use of both baclofen and GHB is associated with serious adverse side effects such as sedation and muscle relaxation, which has significantly hampered their therapeutic value in the treatment of mental health disorders (Gassmann et al. 2012; Fritzius et al. 2019).

The molecular diversity of functional GBR is defined by GABA_{B1a} (GB1a), GABA_{B1b} (GB1b) and GABA_{B2} (GB2) subunits, which can form either heterodimeric GB1a/2 or GB1b/2 receptor subtypes (Bettler et al. 2006; Bettler et al. 2004). Most importantly, GB1a and GB1b fulfil different physiological roles resulting from distinct cellular localizations (Vigot et al. 2006). GB1a and GB1b subunits are nearly identical except for a 129-residue extension at the extreme extracellular N-terminus of GB1a, referred to as the sushi domains (SDs) (Blein et al. 2004). Previous studies indicate that the major difference between the two isoforms is that they display very distinct subcellular distribution, where the GB1a/2 receptor localizes to presynaptic sites, whereas the GB1b/2 receptor localizes to postsynaptic sites (Ulrich and Bettler 2007; Perez-Garci et al. 2006). Consequently, GB1a/2 is involved in regulating vesicular neurotransmitter release (including glutamate) at presynaptic termini, while GB1b/2 activates potassium currents in the spines and dendrites.

Recent proteomic studies identified three transmembrane proteins interacting specifically with the GB1a/2 receptor: the amyloid precursor protein (APP), the adherence-junction associated protein 1 (AJAP1) and the PILRα-associated neural protein (PIANP) (Schwenk et al. 2016; Dinamarca et al. 2019; Rice et al. 2019). Sequence alignment of the APP, AJAP1 and PIANP sequences displayed no obvious similarity, except for a stretch of six amino acids, suggesting a potential central binding interface, which mediates the interaction of these proteins with the N-terminal Sushi domain 1 (SD1) of GBR1a/2. Interestingly, the affinity for SD1 binding is different within the three SD-interacting proteins and follows the order AJAP1>PIANP>>APP. Functionally, APP mediates the trafficking of GB1a/2 receptors to the presynaptic site, where GB1a/2 receptors are likely transferred to AJAP1 or PIANP that precisely localize the receptor at the cell surface. Activated presynaptic GBRs at glutamatergic terminals inhibit the release of neurotransmitters (glutamate) and thereby inhibit excitatory neuronal transmission.

Mild cognitive impairment (MCI) is a stage of cognitive decline between normal aging caused cognitive decline and dementia. Consequently, patients suffering from MCI have difficulties with memory, language, thinking or judgment. Worldwide, the annual prevalence of MCI in persons older than 60 years is estimated to be between 12-18%. Additionally, among MCI patient groups approx. 10-15% develop dementia annually, and dementia is reported as the seventh leading cause of death worldwide. Currently there is no approved treatment directed at MCI.

Cognitive functions like learning and memory depend on balanced-neuronal transmission. An increase in glutamate or acetylcholine release at excitatory synapses is expected to have cognition enhancing effects in Alzheimer's disease and patients with MCI (Fritzius et al. 2019). Therefore, increasing excitatory neurotransmission in patients with cognition decline may offer a new mode of action to target cognitive impairments, and there is a need for new agents that can modulate excitatory neurotransmission.

### SUMMARY

The current invention is directed to compounds that inhibit GBR-associated protein-protein interactions (PPIs), specifically those involving the N-terminal sushi domains (SDs), and in particular Sushi domain 1 (SD1), of the GABA_{B1a/2} (GB1a/2) receptor, which is present at excitatory terminals.

The compounds disclosed herein are capable of inhibiting protein-protein interactions such as interactions between any one of adherence-junction associated protein 1 (AJAP1), PILRα-associated neural protein (PIANP) and amyloid precursor protein (APP) and the N-terminal SD1 of the GB1a/2 receptor. Interactions with these proteins stabilize presynaptic GB1a/2 receptors at glutamatergic terminals, which controls inhibition of excitatory neurotransmitter release. The compounds of the present disclosure can thus upregulate excitatory neurotransmitter release, resulting in cognition enhancing effects. In particular, the compounds disclosed herein can uncouple presynaptic GB1a/2 from APP, AJAP1 and PIANP and therefore cause delocalization of the receptors and prevent inhibition of neurotransmitter release.

Consequently, this will lead to an increase in excitatory neurotransmission and an enhancement of cognitive functions.

The developed peptide-based modulators act specifically at presynaptic GB1a/2 receptors, resulting in increased efficacy and specificity, and reduced adverse effects.

Thus, one aspect of the present disclosure relates to a peptide comprising or consisting of the sequence 1 2 3 4 5 6 7 8 9 10 11 12 13 14 15

(SEQ ID NO: 64)
E X₁ X₂ X₃ X₄ X₅ X₆ W X₇ X₈ X₉ X₁₀ D X₁₁ D

wherein
X₁ is Aspartic acid (D) or Threonine (T),
X₂ is Aspartic acid (D) or Glutamic acid (E),
X₃ is S, Aspartic acid (D), Tyrosine (Y) or Phenylalanine (F),
X₄ is absent, Tryptophan (W), 3-(2-naphthyl)-L-alanine (2Nala), O-ethyl-L-tyrosine (Oetyr), O-benzyl-L-tyrosine (Obtyr), or L-Cysteic acid (Cya),
X₅ is Isoleucine (I) or Threonine (T),
X₆ is Aspartic acid (D), Tryptophan (W) or Alanine (A),
X₇ is Tyrosine (Y) or Glycine (G),
X₈ is Glycine (G), Proline (P) or Aspartic acid (D),
X₉ is Alanine (A), Threonine (T) or Serine(S),
X₁₀ is absent, Glycine (G), Serine(S) or Alanine (A),
X₁₁ is Aspartic acid (D), Threonine (T) or Glutamic acid (E),
or a functional variant thereof, wherein the functional variant comprises 1 or 2 individual amino acid substitution.

Another aspect of the present disclosure relates to a peptide defined herein for use as a medicament.

Another aspect of the present disclosure relates to a peptide defined herein for use in the treatment of neurological and/or psychiatric disorders, such as wherein the neurological disorder is epilepsy, spasticity, schizophrenia, dystonia, neuropathic pain, narcolepsy, spasticity, cognitive deficits such as Mild cognitive impairment (MCI), autism spectrum disorder and/or substance-use disorders; and/or wherein the psychiatric disorder is depression, anxiety and/or Attention deficit hyperactivity disorder (ADHD).

### DESCRIPTION OF DRAWINGS

FIG. 1: Ala scan of APP_{WT}, AJAP-1_{WT} and PIANP_{WT}. A-C) Affinity fold-change of Ala scan variants of APP_{WT} (A), AJAP-1_{WT} (B) and PIANP_{WT} (C). D) Alignment and positions numbering of APP_{WT}, AJAP-1_{WT} and PIANP_{WT}. All presented peptides have an acetylated N-terminal amino acid and a C-terminal amide.

FIG. 2: In silico ncAAs-based positional scanning of APP_{WT}, AJAP-1_{WT} and PIANP_{WT}. A-C) The heat maps of computationally predicted relative affinity of ncAAs deep mutational scanning arrays of APP_{WT} (A), AJAP-1_{WT} (B) and PIANP_{WT} (C) based on the obtained cAAs deep mutational scanning results. D) Overview of synthesized ncAAs incorporated APP_{WT} variants including the abbreviations of introduced ncAAs and calculated Kᵢ values. All synthesized peptides have an acetylated N-terminal amino acid and a C-terminal amide.

FIG. 3: Characterization of peptides in pull-down assay using the homogenized membrane fraction of wild type mouse brains. AJAP-1_{WT} and designed high affinity peptide 20 are capable of pulling down native GB1a while the control groups including beads alone and scrambled version peptide of 20 (Scr-20) exhibit no enrichment of this protein as shown by immunoblotting.

FIG. 4: Evaluation and optimization of cell membrane permeability and plasma stability of the designed peptides. A-B) Comparison of SD1/2 binding affinity (FP inhibition assay based calculated Kᵢ, mean±SEM, n≥3) of 16 (A) and 17 (B) tagged with poly R of different lengths from 8 R to 3 R. C-D) Comparison of cell membrane permeability (CAPA assay based calculated CP₅₀) of 16 (C) and 17 (D) tagged with poly R of different lengths from 8 R to 3 R and chloroalkane (CA) at the N-terminal. E-F) Comparison of in vitro human plasma stability (expressed as percentage of remaining peptides after 48 h endpoint incubation in human plasma) of 16 (E) and 17 (F) tagged with poly R of different lengths from 8 R to 3 R. G-H) Comparison of the effects of introduced ncAA (2Nala) on the cell membrane permeability (G) (CAPA assay based calculated CP50, mean±SEM, n≥3) and SD1/2 binding affinity (H) (FP inhibition assay based calculated Kᵢ, mean±SEM, n≥3) of 4 R tagged 16, 17, 20 and 21.

FIG. 5: High-affinity peptides efficiently abolish the negative allosteric properties of AJAP-1 at transcellular GB1a/2 receptors. A) Assay monitoring PLC dependent FLuc (firefly luciferase) expression under control of the serum response element (SRE). GB1a/2 receptors were artificially coupled to PLC by stably expressing the chimeric G protein subunit Gα_{q/i}. GB1a/2 and SRE-FLuc reporter were transiently expressed in HEK293T-Gαqi cells. B) Peptides (AJAP-1_{WT}, 16 and 20) at 10 μM significantly alter dose-response curves of GABA-induced FLuc activity in transfected cells in the presence of AJAP-1 in trans (AJAP-1 expressed in neighboring cells). C) The addition of 10 μM AJAP-1_{WT}, 16 or 20 elevate the basal activity of GB1a/2 receptors in presence of AJAP-1 in trans. D) The addition of 10 μM AJAP-1_{WT}, 16 or 20 decreased the EC₅₀ of GABA for activating GB1a/2 receptors in presence of AJAP-1 in trans. E) The addition of 10 μM AJAP-1_{WT}, 16 or 20 did not alter the maximum efficacy (Eₘₐₓ) of GABA for activating GB1a/2Rs in presence of AJAP-1 in trans. Note: the vehicle is PBS.

FIG. 6: High-affinity peptide efficiently abolishes AJAP1-mediated recruitment of GB1a/2 receptors at neuron's membrane. A) Confocal fluorescent imaging of AJAP-1 and GBRs using AJAP-1 transfected HEK293T/mouse hippocampal neuron co-cultures. B) The quantification of the fluorescence intensity of GBRs at the neuronal membrane reveals a significant reduction in membrane abundance when exposed to the high-affinity peptide 20 compared to the negative control scrambled peptide Scr-20. Furthermore, the inhibitory effect becomes increasingly significant with extended incubation time at 30 minutes and 60 minutes relative to at 10 min.

### DEFINITIONS

The term “affinity” refers to the strength of binding between a ligand and its receptor.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed herewith: Y, G, F, M, A, S, I, L, T, V, P, K, H, Q, E, W, R, D, N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” as applied herein, one amino acid may be substituted for another, in one embodiment, within the groups of amino acids indicated herein below:

Amino acids having polar side chains (Asp, Glu, Lys, Arg, His, Asn, Gln, Ser, Thr, Pro, and Cys); Amino acids having non-polar side chains (Gly, Ala, Val, Leu, Ile, Phe, Trp, Tyr and Met); Amino acids having aliphatic side chains (Gly, Ala Val, Leu, Ile); Amino acids having cyclic side chains (Trp, His, Pro); Amino acids having aromatic side chains (Phe, Tyr, Trp); Amino acids having acidic, such as negatively charged side chains (Asp, Glu); Amino acids having basic, such as positively charged side chains (Lys, Arg, His); Amino acids having amide side chains (Asn, Gln); Amino acids having hydroxy side chains (Ser, Thr); Amino acids having sulphur-containing side chains (Cys, Met); Neutral, weakly hydrophobic amino acids (Pro, Ala, Gly, Ser, Thr); Hydrophilic, acidic amino acids (Gln, Asn, Glu, Asp); and Hydrophobic amino acids (Leu, Ile, Val).

Where the L or D form (optical isomers) has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

A “functional variant” of a peptide is a peptide capable of performing essentially the same functions as the peptide it is a functional variant of. In particular, a functional variant can bind the same molecules, preferably with the same affinity, as the peptide it is a functional variant of.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention.

The term “individual” refers to vertebrates, particular members of the mammalian species, preferably primates including humans. As used herein, ‘subject’ and ‘individual’ may be used interchangeably. Treatment of animals, such as mice, rats, dogs, cats, cows, horses, sheep and pigs, is, however, also within the scope of the present invention.

An “individual in need thereof” refers to an individual who may benefit from treatment. In one embodiment, said individual in need thereof is a diseased individual, wherein said disease may be a neurological and/or a psychiatric disorder.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

A treatment according to the invention can be prophylactic, ameliorating and/or curative.

“Pharmacologically effective amount”, “pharmaceutically effective amount” or “physiologically effective amount” of a “bioactive agent” is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered.

### DETAILED DESCRIPTION

### Peptides

One aspect of the present disclosure relates to a peptide comprising or consisting of the sequence 1 2 3 4 5 6 7 8 9 10 11 12 13 14 15

(SEQ ID NO: 64)
E X₁ X₂ X₃ X₄ X₅ X₆ W X₇ X₈ X₉ X₁₀ D X₁₁ D

wherein
X₁ is Aspartic acid (D) or Threonine (T),
X₂ is Aspartic acid (D) or Glutamic acid (E),
X₃ is S, Aspartic acid (D), Tyrosine (Y) or Phenylalanine (F),
X₄ is absent, Tryptophan (W), 3-(2-naphthyl)-L-alanine (2Nala), O-ethyl-L-tyrosine (Oetyr), O-benzyl-L-tyrosine (Obtyr), or L-Cysteic acid (Cya),
X₅ is Isoleucine (I) or Threonine (T),
X₆ is Aspartic acid (D), Tryptophan (W) or Alanine (A),
X₇ is Tyrosine (Y) or Glycine (G),
X₈ is Glycine (G), Proline (P) or Aspartic acid (D),
X₉ is Alanine (A), Threonine (T) or Serine(S),
X₁₀ is absent, Glycine (G), Serine(S) or Alanine (A),
X₁₁ is Aspartic acid (D), Threonine (T) or Glutamic acid (E),
or a functional variant thereof, wherein the functional variant comprises 1 or 2 individual amino acid substitutions.

One aspect of the present disclosure relates to a peptide comprising or consisting of an amino acid sequence disclosed in any one of Table 3, Table 4, Table 5, Table 6, Table 7, Table 8, Table 9, Table 10, Table 11, Table 12, Table 13, Table 14, Table 15 or Table 16, as well as functional variant thereof, wherein the functional variant comprises 1 or 2 individual amino acid substitutions.

In one embodiment of the present disclosure, the functional variant of the peptides disclosed herein comprises 2 individual amino acid substitutions.

In one embodiment of the present disclosure, the functional variant of the peptides disclosed herein comprises 1 individual amino acid substitution.

In one embodiment of the present disclosure, the peptides or functional variants thereof comprises at least 8 amino acid residues, such as at least 9 amino acid residues, such as at least 10 amino acid residues, such as at least 11 amino acid residues, such as at least 12 amino acid residues, such as at least 13 amino acid residues, such as at least 14 amino acid residues, such as at least 15 amino acid residues, such as between 8 and 35 amino acid residues, such as between 8 and 30 amino acid residues, such as between 8 and 27 amino acid residues, such as between 8 and 23 amino acid residues.

In one embodiment of the present disclosure, the peptides or functional variants thereof comprises no more than 35 amino acid residues, such as no more than 34 amino acid residues, such as no more than 33 amino acid residues, such as no more than 32 amino acid residues, such as no more than 31 amino acid residues, such as no more than 30 amino acid residues, such as no more than 29 amino acid residues, such as no more than 28 amino acid residues, such as no more than 27 amino acid residues, such as no more than 26 amino acid residues, such as no more than 25 amino acid residues, such as no more than 24 amino acid residues, such as no more than 23 amino acid residues, such as no more than 22 amino acid residues, such as no more than 21 amino acid residues, such as no more than 20 amino acid residues.

In one embodiment of the present disclosure, the peptides or functional variants thereof consists of at least 8 amino acid residues. In one embodiment of the present disclosure, the peptides or functional variants thereof consists of 8 amino acid residues, such as 9 amino acid residues, such as 10 amino acid residues, such as 11 amino acid residues, such as 12 amino acid residues, such as 13 amino acid residues, such as 14 amino acid residues, such as 15 amino acid residues, such as 16 amino acid residues, such as 17 amino acid residues, such as 18 amino acid residues, such as 19 amino acid residues, such as 20 amino acid residues, such as 21 amino acid residues, such as 22 amino acid residues, such as 23 amino acid residues, such as 24 amino acid residues, such as 25 amino acid residues, such as 26 amino acid residues, such as 27 amino acid residues.

In one embodiment of the present disclosure, X₁ of the peptides disclosed herein is D.

In one embodiment of the present disclosure, X₂ of the peptides disclosed herein is D.

In one embodiment of the present disclosure, X₃ of the peptides disclosed herein is S, D or Y. In one embodiment of the present disclosure, X₃ of the peptides disclosed herein is D or Y. In one embodiment of the present disclosure, X₃ of the peptides disclosed herein is X₃ is D.

The present inventors have found that residues at positions 5 to 8, corresponding to X₄ X₅ X₆ W, of the peptides disclosed herein form most interactions with SD1, and these interactions are facilitated by the presence of hydrophobic amino acid residues at one or more of these positions. Hence, presence of hydrophobic amino acid residues at one or more of these positions makes the peptides of the present disclosure have high affinity to SD1 and to GB1a/2Rs.

Thus, in one embodiment one or more of the amino acid residues at positions 5 to 8 of the peptides of the present disclosure, or functional variants thereof, are hydrophobic residues.

In one embodiments one or both of the amino acid residues at positions 6 and 8 of the peptides of the present disclosure, or functional variants thereof, are hydrophobic residues.

In one embodiment of the present disclosure, X₄ of the peptides disclosed herein is absent, or it is W, 2-Nala, Oetyr or Obtyr. Presence of an unnatural amino acid at position 5, such as at X₄ may improve affinity. Presence of an unnatural amino acid at position 5, such as at X₄ may improve stability of the peptide to proteases.

In one embodiment of the present disclosure, X₄ of the peptides disclosed herein is absent.

In one embodiment of the present disclosure, X₄ of the peptides disclosed herein is W, 2-Nala, Oetyr or Obtyr. In one embodiment of the present disclosure, X₄ of the peptides disclosed herein is W or 2-Nala.

In one embodiment of the present disclosure, X₅ of the peptides disclosed herein is I.

In one embodiment of the present disclosure, X₅ of the peptides disclosed herein is T.

In one embodiment of the present disclosure, X₆ of the peptides disclosed herein is D.

The present inventors have also found that the amino acid residue at position 10, corresponding to X₈, is involved in pi-pi interactions with certain residues of SD1, thus, presence of suitable residues at this position may also be important for the affinity of the peptides of the present disclosure to SD1 and to GB1a/2Rs.

Thus, in one embodiment of the present disclosure, X₈ of the peptides disclosed herein is P or D.

In one embodiment of the present disclosure, X₉ of the peptides disclosed herein is A or S, and X₁₀ is absent.

In one embodiment of the present disclosure, X₉ of the peptides disclosed herein is T and X₁₀ is G or S.

In one embodiment of the present disclosure, X₁₀ of the peptides disclosed herein is G, S or absent. In one embodiment of the present disclosure, X₁₀ of the peptides disclosed herein is G or S. In one embodiment of the present disclosure, X₁₀ is absent.

In one embodiment of the present disclosure, X₁₀ of the peptides disclosed herein is absent and X₁₁ is D or T. In one embodiment of the present disclosure, X₁₀ of the peptides disclosed herein is absent and X₁₁ is D.

In one embodiment of the present disclosure, X₁₁ of the peptides disclosed herein is D or E and X₁₀ is G or S.

In one embodiment of the present disclosure, the peptide is selected from the group consisting of:

SEQ ID NO: 15:
TETEFIAWGPTGDED
SEQ ID NO: 16:
EDDDWIDWYDSDDD
SEQ ID NO: 17:
EDDDWIDWGPTSDDD
SEQ ID NO: 18:
EDDYIDWYDSDDD
SEQ ID NO: 19:
EDDYIDWGPTSDDD
SEQ ID NO: 20:
EDDD(2Nala)IDWYDSDDD
SEQ ID NO: 21:
EDDD(2Nala)IDWGPTSDDD
SEQ ID NO: 22:
EDDYTDWYDSDDD
SEQ ID NO: 23:
EDDYTDWGPTSDDD
SEQ ID NO: 24:
ETEFIAWGPTADED
SEQ ID NO: 25:
ETEYIAWGPTGDED
SEQ ID NO: 26:
ETEFIDWGPTGDED
SEQ ID NO: 27:
EDDSWIWWGPTGDED
SEQ ID NO: 28:
EDDSWIWWYGADTD
SEQ ID NO: 29:
EDDSWIDWGPTGDED
SEQ ID NO: 30:
EDDSWIDWYGADTD
SEQ ID NO: 31:
ETEFI(Cya)WGPTGDED
SEQ ID NO: 32:
EDDD(Oetyr)IDWYDSDDD
SEQ ID NO: 33:
EDDD(Obtyr)IDWYDSDDD
SEQ ID NO: 34:
EDDD(Oetyr)IDWGPTSDDD
SEQ ID NO: 35:
EDDD(Obtyr)IDWGPTSDDD

or a functional variant thereof, wherein the functional variant comprises 1 or 2 individual amino acid substitutions.

In one embodiment of the present disclosure, the peptide is N-terminally acetylated (Ac—N-terminus).

In one embodiment of the present disclosure, the peptide is C-terminally amidated (—NH₂).

In one embodiment of the present disclosure, the C-terminus of any of the peptides disclosed herein is a carboxylic acid.

The trafficking process of GB1a-containing receptors along the axon localizes intracellularly, thus the cell membrane represents a significant barrier to be crossed by peptides that are intended to modulate GB1a-containing receptors and in particular to target the interaction between certain proteins and SD1/2, such as between any of APP, AJAP-1 and PIANP and SD1/2.

Thus, in one embodiment of the present disclosure, the peptide disclosed herein is conjugated to a cell-penetrating peptide (CPP). Presence of a CPP may facilitate both cell penetration and permeability through the blood brain barrier.

In one embodiment of the present disclosure, the CPP may be conjugated to the N-terminus or to the C-terminus of a peptide of the present disclosure. In one embodiment of the present disclosure, the CPP may be conjugated to the N-terminus of a peptide of the present disclosure.

In one embodiment of the present disclosure, the CPP may be any one of:

Tat,
(SEQ ID NO: 55)
YGRKKRRQRRR,
R8,
(SEQ ID NO: 56)
RRRRRRRR,
R7,
(SEQ ID NO: 57)
RRRRRRR,
R6,
(SEQ ID NO: 58)
RRRRRR,
R5,
(SEQ ID NO: 59)
RRRRR,
R4,
(SEQ ID NO: 60)
RRRR,
R3,
RRR,
or
R2,
RR.

Longer CPP may improve cell permeability of a peptide of the present disclosure. However, too long CPP may negatively affect affinity of a peptide of the present disclosure for SD1/2.

In one embodiment of the present disclosure, the CPP is

R8,
(SEQ ID NO: 56)
RRRRRRRR
or
R4,
(SEQ ID NO: 60)
RRRR

In one embodiment of the present disclosure, the CPP may conjugated to a peptide of the present disclosure via a linker, for example via a peptidic linker.

In one embodiment of the present disclosure, the peptide is selected from the group consisting of:

SEQ ID NO: 15:
Ac-TETEFIAWGPTGDED-NH₂
SEQ ID NO: 16:
Ac-EDDDWIDWYDSDDD-NH₂
SEQ ID NO: 17:
Ac-EDDDWIDWGPTSDDD-NH₂
SEQ ID NO: 18:
Ac-EDDYIDWYDSDDD-NH₂
SEQ ID NO: 19:
Ac-EDDYIDWGPTSDDD-NH₂
SEQ ID NO: 20:
Ac-EDDD(2Nala)IDWYDSDDD-NH₂
SEQ ID NO: 21:
Ac-EDDD(2Nala)IDWGPTSDDD-NH₂
SEQ ID NO: 22:
Ac-EDDYTDWYDSDDD-NH₂
SEQ ID NO: 23:
Ac-EDDYTDWGPTSDDD-NH₂
SEQ ID NO: 24:
Ac-ETEFIAWGPTADED-NH₂
SEQ ID NO: 25:
Ac-ETEYIAWGPTGDED-NH₂
SEQ ID NO: 26:
Ac-ETEFIDWGPTGDED-NH₂
SEQ ID NO: 27:
Ac-EDDSWIWWGPTGDED-NH₂
SEQ ID NO: 28:
Ac-EDDSWIWWYGADTD-NH₂
SEQ ID NO: 29:
Ac-EDDSWIDWGPTGDED-NH₂
SEQ ID NO: 30:
Ac-EDDSWIDWYGADTD-NH₂
SEQ ID NO: 31:
Ac-ETEFI(Cya)WGPTGDED-NH₂
SEQ ID NO: 32:
Ac-EDDD(Oetyr)IDWYDSDDD-NH₂
SEQ ID NO: 33:
Ac-EDDD(Obtyr)IDWYDSDDD-NH₂
SEQ ID NO: 34:
Ac-EDDD(Oetyr) IDWGPTSDDD-NH₂
SEQ ID NO: 35:
Ac-EDDD(Obtyr)IDWGPTSDDD-NH₂
SEQ ID NO: 36:
Ac-RRRRRRRREDDDWIDWYDSDDD-NH₂
SEQ ID NO: 37:
Ac-RRRREDDDWIDWYDSDDD-NH₂
SEQ ID NO: 38:
Ac-RRREDDDWIDWYDSDDD-NH₂
SEQ ID NO: 39:
Ac-RRRRRRRREDDDWIDWGPTSDDD-NH₂
SEQ ID NO: 40:
Ac-RRRREDDDWIDWGPTSDDD-NH₂
SEQ ID NO: 41:
Ac-RRREDDDWIDWGPTSDDD-NH₂
SEQ ID NO: 42:
Ac-RRRREDDD(2Nala)IDWYDSDDD-NH₂
SEQ ID NO: 43:
Ac-RRRREDDD(2Nala)IDWGPTSDDD-NH₂

or a functional variant thereof, wherein the functional variant comprises 1 or 2 individual amino acid substitutions.

In one embodiment of the present disclosure, the peptide is conjugated to an additional moiety.

### Activities of the Peptides

In one embodiment, the present disclosure relates to peptides that are inhibitors of GB1a/2Rs (heterodimeric GABA_{B1a}/GABA_{B2} receptor).

In one embodiment, the present disclosure relates to peptides that are capable of modulating excitatory neurotransmission.

GB1a/2Rs display very distinct subcellular distribution compared to GB1b/2Rs, and in particular GB1a/2Rs are localized at presynaptic sites, whereas GB1b/2Rs accumulate at postsynaptic terminals. Consequently, the peptides disclosed herein inhibit GB1a/2Rs at presynaptic sites and in particular inhibit activation of GB1a/2Rs at presynaptic sites. Consequently, the peptides of the present disclosure may enhance excitatory neuronal transmission by preventing and/or stopping GB1a/2Rs from inhibiting the release of neurotransmitters such as glutamate.

In particular, in one embodiment, the peptides of the present disclosure, as well as functional variants thereof, are capable of inhibiting an interaction between a protein and a sushi domain 1/2 (SD1/2) of GB1a, such as an interaction between SD1 of GB1a at presynaptic neurons.

In one embodiment, the peptides of the present disclosure, as well as functional variants thereof, are capable of inhibiting an interaction between SD1 of GB1a, such as an interaction between SD1 of GB1a at presynaptic neurons.

In one embodiment, the peptides of the present disclosure, as well as functional variants thereof, are capable of inhibiting an interaction between:
SD and adherence-junction associated protein 1 (AJAP1),
SD and PILRα-associated neural protein (PIANP), and/or
SD and amyloid precursor protein (APP).

In one embodiment, the peptides of the present disclosure, as well as functional variants thereof, have binding affinity (Kᵢ) to a sushi domain 1/2 (SD1/2) protein of 20 nM or less.

A definition of SD1/2 is found in Dinamarca et al. 2019.

For example, the peptides of the present disclosure, as well as functional variants thereof, have higher binding affinity (Kᵢ) to SD1/2 than wild-type AJAP-1 (SEQ ID NO: 15).

In one embodiment, the peptides of the present disclosure, as well as functional variants thereof, have binding affinity (Kᵢ) to a sushi domain 1/2 (SD1/2) protein of 19 nM or less, such as of 18 nM or less, such as of 17 nM or less, such as of 16 nM or less, such as of 15 nM or less, such as of 14 nM or less, such as of 13 nM or less, such as of 12 nM or less, such as of 11 nM or less, such as of 10 nM or less, such as of 9 nM or less, such as of 8 nM or less, such as of 7 nM or less, such as of 6 nM or less, such as of 5 nM or less, such as of 4 nM or less, such as of 3 nM or less, such as of 2 nM or less, such as of about 1 nM.

Binding affinity can be measured according to any conventional methods for determining affinity of a peptide or ligand to its receptor known by the skilled person, e.g. those described in the working examples.

As provided herein above, trafficking process of GB1a-containing receptors along the axon localizes intracellularly, and therefore a peptide needs to penetrate into a cell in order to bind to those receptors.

Thus, in one embodiment, the peptides of the present disclosure, as well as functional variants thereof, have membrane permeability (CP₅₀) of 30 μM or less, such as 25 μM or less, such as 20 μM or less, such as 15 μM or less, such as 10 μM or less. Permeability of a peptide can be measured according to any conventional methods of for determining permeability of a peptide into a mammalian cell known by the skilled person, e.g. those described in the working examples.

In one embodiment, the peptides of the present disclosure, as well as functional variants thereof, have membrane permeability (CP₅₀) of 28 μM or less, such as of 25 μM or less, such as of 20 μM or less, such as of 15 μM or less, such as of 10 μM or less, such as of 5 μM or less, such as about 1 μM.

In one embodiment, the peptides of the present disclosure, as well as functional variants thereof, have plasma stability (T_{1/2}) of 12 hours or more, such as of 14 hours or more, such as of 16 hours or more, such as of 18 hours or more, such as of 20 hours or more, such as of 22 hours or more, such as of 24 hours or more, such as of 26 hours or more, such as of 28 hours or more, such as of 30 hours or more, such as of 32 hours or more, such as of 34 hours or more, such as of 38 hours or more, such as of 40 hours or more, such as of 42 hours or more, such as of 44 hours or more, such as of 46 hours or more, 48 hours or more.

Stability of a peptide, e.g. metabolic stability and/or plasma stability of a peptide, can be measured according to any conventional methods of for determining stability of a peptide in a mammalian system known by the skilled person, e.g. those described in the working examples.

In one embodiment, the peptides of the present disclosure, as well as functional variants thereof, cause an increase in the constitutive activity and a decrease in the EC₅₀ of GABA towards GB1a/2R, thus reducing the negative allosteric modulation effects of any one of AJAP-1 and/or PIANP at GB1a/2Rs.

### Cyclic Peptides

In one embodiment, the peptide is cyclized to form a cyclic polypeptide. For example, a peptide may be cyclized by side chain-to-side chain, tail-to-side chain, side chain-to-head and head-to-tail. It will be evident to the skilled person that the peptides of the present disclosure can be made cyclic by any method known in the art. Common cyclization strategies include, but are not limited to, disulfide bridge between two cysteines (side chain-to-side chain), thioether bridge with e.g. a bromoacetic addition on the N-terminus and a cysteine (head-to-side chain) and lactamization either using coupling between a basic residue (Lys) and acid residues (Asp or Glu), or via native chemical ligation (NCL).

In one embodiment, the peptide is back-bone cyclized. In one embodiment, the peptide is “head-to-tail” cyclized, i.e. the N-terminal amino acid residue is linked to the C-terminal amino acid residue. In one embodiment, the N-terminal amino acid residue is linked to the C-terminal amino acid residue via a peptide bond. In one embodiment, the N-terminal amino acid residue is linked to the C-terminal amino acid residue via a non-peptide chemical linker.

Thus, in one embodiment, the peptides of the present disclosure further comprise:
a. two cysteines linked via a non-peptide chemical linker; and/or
b. a cysteine linked to the N-terminal amino acid residue of the peptide.

In one embodiment, the peptides of the present disclosure further comprise two cysteines.

In one embodiment, the peptides of the present disclosure further comprise a cysteine at the N-terminal. In one embodiment, the peptides of the present disclosure further comprise a cysteine at the C-terminal. In one embodiment, the peptides of the present disclosure further comprise a cysteine at the N-terminal and a cysteine at the C-terminal.

In one embodiment, a linker connects two cysteines within the peptide of the present disclosure.

In one embodiment, a linker connects a cysteine at the N-terminal amino acid residue and a cysteine at the C-terminal amino acid residue of the peptides of the present disclosure.

In one embodiment, the non-peptide chemical linker is:

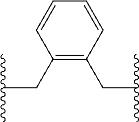 [SMILES: *Cc1ccccc1C*]

Thus, in one embodiment, the peptide of the present disclosure is selected from the group consisting of:

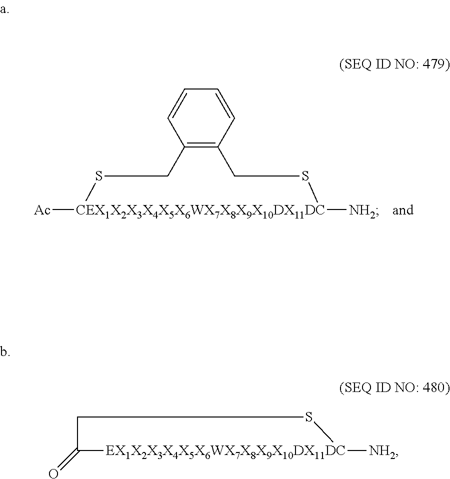

wherein X₁ to X₁₁ are defined as in the section “Peptides” of the present disclosure.

Thus, in one embodiment, the peptide of the present disclosure is

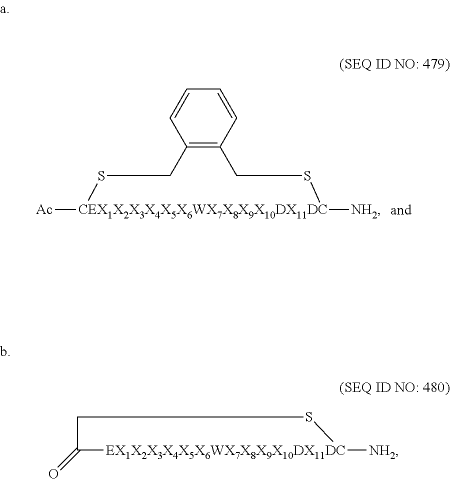

wherein X₁ to X₁₁ are defined as in the section “Peptides” of the present disclosure.

In one embodiment, the peptide of the present disclosure is

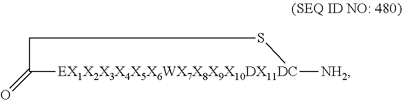

wherein X₁ to X₁₁ are defined as in the section “Peptides” of the present disclosure.

Thus, in one embodiment, the peptide of the present disclosure is selected from the group consisting of:

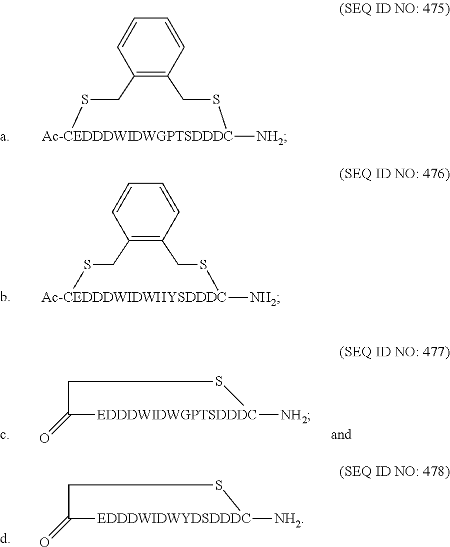

Thus, in one embodiment, the peptide of the present disclosure is selected from the group consisting of:

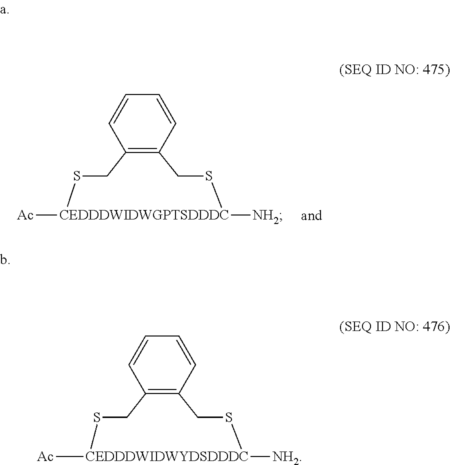

Thus, in one embodiment, the peptide of the present disclosure is selected from the group consisting of:

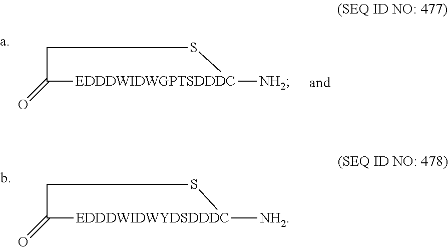

In one embodiment, the peptide of the present disclosure is

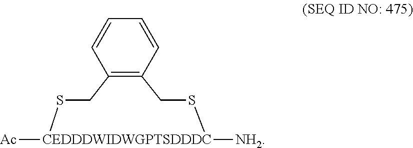

In one embodiment, the peptide of the present disclosure is

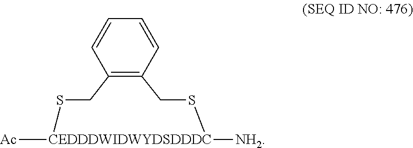

In one embodiment, the peptide of the present disclosure is

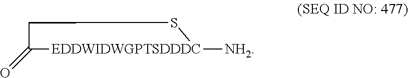

In one embodiment, the peptide of the present disclosure is

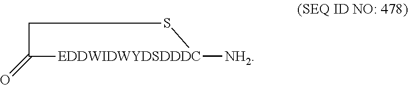

### Medicament/Medical Use

One aspect of the present disclosure relates to a peptide disclosed herein, as well as functional variants thereof, for use as a medicament.

The present inventors have found that the peptides of the present disclosure, as well as functional variants thereof, can act directly at presynaptic site and modulate the release of several excitatory neurotransmitters including glutamate and acetylcholine. This finds application in treatment of multiple diseases and disturbs.

Another aspect of the present disclosure relates to a peptide disclosed herein, as well as functional variants thereof, for use in the treatment of neurological and/or psychiatric disorders.

In one embodiment, the neurological disorder is epilepsy, spasticity, schizophrenia, dystonia, neuropathic pain, narcolepsy, spasticity, cognitive deficits such as Mild cognitive impairment (MCI), autism spectrum disorder and/or substance-use disorders.

In one embodiment, the neurological disorder is a cognitive deficit, for example MCI, in an individual suffering from Alzheimer's disease.

In one embodiment, the psychiatric disorder is depression, anxiety and/or Attention deficit hyperactivity disorder (ADHD).

One aspect of the present disclosure relates to a method for treatment of neurological and/or psychiatric disorders, and symptoms thereof, comprising administration of a peptide according to the present disclosure, as well as functional variants thereof, to an individual in need thereof.

One aspect of the present disclosure relates to a use of a peptide according to the present disclosure, as well as functional variants thereof, for the manufacture of a medicament for the treatment of neurological and/or psychiatric disorders, and symptoms thereof.

One aspect of the present disclosure relates to a medicament for use in the treatment or prevention of neurological and/or psychiatric disorders, and symptoms thereof comprising a peptide according to the present disclosure, as well as functional variants thereof.

Without being bound to theory, a peptide of the present disclosure, as well as a functional variant thereof, may be capable of increasing the release of dopamine and/or serotonin in an individual suffering from depressive disorders or ADHD.

### Nucleic Acid Construct Encoding a Peptide

In one embodiment there is provided a nucleic acid construct encoding a peptide as defined herein. In one embodiment said nucleic acid construct will be able to continuously express said peptide for a prolonged period of time; such as at least 1 month, for example at least 2 months, such as at least 3 months, for example at least 4 months, such as at least 5 months, for example at least 6 months, such as at least 7 months, for example at least 8 months, such as at least 9 months, for example at least 12 months.

It is thus an aspect to provide a nucleic acid construct encoding a peptide as defined herewith.

In one embodiment there is provided a nucleic acid construct encoding a peptide of SEQ ID NO: 64, or a functional variant thereof, or any peptide disclosed herein, such as any peptide disclosed in any one of Table 3, table 4, Table 5, Table 6, Table 7, Table 8, Table 9, Table 10, Table 11, Table 12, Table 13, Table 14, Table 15 and/or Table 16, or a functional variant thereof.

It is also an aspect to provide a nucleic acid construct encoding a peptide as defined herein for use in a method of treating neurological and/or psychiatric disorders as defined herein.

By nucleic acid construct is understood a genetically engineered nucleic acid. The nucleic acid construct may be a non-replicating and linear nucleic acid, a circular expression vector or an autonomously replicating plasmid. A nucleic acid construct may comprise several elements such as, but not limited to genes or fragments of same, promoters, enhancers, terminators, poly-A tails, linkers, polylinkers, operative linkers, multiple cloning sites (MCS), markers, STOP codons, internal ribosomal entry sites (IRES) and host homologous sequences for integration or other defined elements. It is to be understood that the nucleic acid construct according to the present invention may comprise all or a subset of any combination of the above-mentioned elements.

Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). Further, nucleic acid constructs according to the present invention may be synthesized without template, and may be obtained from various commercial suppliers (e.g. Genscript Corporation).

In one embodiment, the nucleic acid constructs are naked DNA constructs comprising sequences encoding the triple agonist peptide.

### Delivery Vehicles

It is also an aspect to provide the nucleic acid construct as described herein above comprised within a delivery vehicle. A delivery vehicle is an entity whereby a nucleotide sequence or polypeptide or both can be transported from at least one media to another. Delivery vehicles are generally used for expression of the sequences encoded within the nucleic acid construct and/or for the intracellular delivery of the construct or the polypeptide encoded therein.

In one embodiment, there is provided a delivery vehicle comprising the nucleic acid construct as defined herein. A delivery vehicle may be selected from the group consisting of: RNA based vehicles, DNA based vehicles/vectors, lipid based vehicles (such as a liposome), polymer based vehicles (such as a cationic polymer DNA carrier), colloidal gold particles (coating) and virally derived DNA or RNA vehicles or vectors.

Methods of non-viral delivery include physical (carrier-free delivery) and chemical approaches (synthetic vector-based delivery).

Physical approaches, including needle injection, gene gun, jet injection, electroporation, ultrasound, and hydrodynamic delivery, employ a physical force that permeates the cell membrane and facilitates intracellular gene transfer. Said physical force may be electrical or mechanical.

Examples of chemical delivery vehicles include, but are not limited to: biodegradable polymer microspheres, lipid based formulations such as liposome carriers, cationically charged molecules such as liposomes, calcium salts or dendrimers, lipopolysaccharides, polypeptides and polysaccharides.

### Recombinant Cell

Another aspect of relates to a cell comprising the nucleic acid construct as defined herein. Such a recombinant cell can be used a tool for in vitro research, as a delivery vehicle for the nucleic acid construct or as part of a gene-therapy regime. The nucleic acid construct can be introduced into cells by techniques well known in the art which include microinjection of DNA into the nucleus of a cell, transfection, electroporation, lipofection/liposome fusion and particle bombardment. Suitable cells include autologous and non-autologous cells, and may include xenogenic cells.

### Method of Preparation (Peptide)

In one aspect, the present disclosure relates to methods of manufacturing peptides according to SEQ ID NO: 64 or a functional variant thereof, or any peptide disclosed herein such as any peptide disclosed in any one of Table 3, table 4, Table 5, Table 6, Table 7, Table 8, Table 9, Table 10, Table 11, Table 12, Table 13, Table 14, Table 15 and/or Table 16, or a functional variant thereof.

Peptides according to the present invention may be prepared according to any conventional methods of peptide synthesis known by the skilled person, e.g. those described in the working examples. The starting materials for the processes described in the present application are known or may readily be prepared by conventional methods known by the skilled artisan from commercially available chemicals.

The peptides disclosed herein, as well as functional variants thereof, may be prepared by any methods known in the art; such as by standard peptide-preparation techniques including solution peptide synthesis or Merrifield-type solid phase peptide synthesis.

In one embodiment a peptide according to the disclosure is synthetically made or produced. The methods for synthetic production of peptides are well known in the art.

Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

In one embodiment the peptide or peptide sequences of the disclosure are produced synthetically, in particular, by the sequence assisted peptide synthesis (SAPS) method, by solution synthesis, by solid-phase peptide synthesis (SPPS) such as Merrifield-type solid phase synthesis, by recombinant techniques (production by host cells comprising a first nucleic acid sequence encoding the peptide operably associated with a second nucleic acid capable of directing expression in said host cells) or enzymatic synthesis. These are well-known to the skilled person.

Peptides may be synthesised either batch-wise on a fully automated peptide synthesiser using 9-fluorenylmethyloxycarbonyl (Fmoc) or tert-Butyloxycarbonyl (Boc) as N-alpha-amino protecting group and suitable common protection groups for side-chain functionalities.

After purification such as by reversed phase HPLC, peptides may be further processed to obtain for example C- or N-terminal modified isoforms. The methods for terminal modification are well-known in the art.

### Pharmaceutical Formulations

In one aspect, the present invention relates to a composition comprising the peptide as disclosed herein. Preferably, said composition is pharmaceutically acceptable. In one embodiment the composition as described herein is in the form of a pharmaceutical formulation. In one embodiment, the composition as described herein further comprises a pharmaceutically acceptable carrier. In one aspect, the present invention concerns a composition comprising the compound as defined herein and a pharmaceutically acceptable carrier.

### Items

1. A peptide comprising or consisting of the sequence
   1 2 3 4 5 6 7 8 9 10 11 12 13 14 15

(SEQ ID NO: 64)
E X₁ X₂ X₃ X₄ X₅ X₆ W X₇ X₈ X₉ X₁₀ D X₁₁ D

wherein
X₁ is Aspartic acid (D) or Threonine (T),
X₂ is Aspartic acid (D) or Glutamic acid (E),
X₃ is S, Aspartic acid (D), Tyrosine (Y) or Phenylalanine (F),
X₄ is absent, Tryptophan (W), 3-(2-naphthyl)-L-alanine (2Nala), O-ethyl-L-tyrosine (Oetyr), O-benzyl-L-tyrosine (Obtyr), or L-Cysteic acid (Cya),
X₅ is Isoleucine (I) or Threonine (T),
X₆ is Aspartic acid (D), Tryptophan (W) or Alanine (A),
X₇ is Tyrosine (Y) or Glycine (G),
X₈ is Glycine (G), Proline (P) or Aspartic acid (D),
X₉ is Alanine (A), Threonine (T) or Serine(S),
X₁₀ is absent, Glycine (G), Serine(S) or Alanine (A),
X₁₁ is Aspartic acid (D), Threonine (T) or Glutamic acid (E),
or a functional variant thereof, wherein the functional variant comprises 1 amino acid substitution.
2. The peptide according to item 1, wherein said peptide is an inhibitor of GB1a/2Rs (heterodimeric gamma-amino butyric acid (GABA)_{B1a}/GABA_{B2} receptor), such as wherein said peptide is an inhibitor of protein-protein interactions at GB1a/2Rs.
3. The peptide according to anyone of the preceding items, wherein:
   a. X₁ is D;
   b. X₂ is D;
   c. X₃ is S, D or Y, such as wherein X₃ is D or Y, such as wherein X₃ is D;
   d. X₄ is absent, W, 2-Nal, O-Ey-Y or Obtyr; wherein X₄ is absent; or wherein X₄ is W, 2-Nal, O-Ey-Y or Obtyr, such as wherein X₄ is W or 2-Nal;
   e. X₅ is I;
   f. X₆ is D; and/or
   g. X₈ is P or D,
or a functional variant thereof, wherein the functional variant comprises 1 amino acid substitution.
4. The peptide according to anyone of the preceding items, wherein X₉ is A or S, and X₁₀ is absent; or wherein X₉ is T and X₁₀ is G, S.
5. The peptide according to anyone of the preceding items, wherein X₁₀ is G, S or absent, such as wherein X₁₀ is G or S, such as wherein X₁₀ is absent.
6. The peptide according to anyone of the preceding items, wherein X₁₀ is absent and wherein X₁₁ is D or T, such as wherein X₁₀ is absent and wherein X₁₁ is D; or wherein X₁₁ is D or E and wherein X₁₀ is G, S.
7. The peptide according to anyone of the preceding items, wherein the peptide is selected from the group consisting of:

SEQ ID NO: 15:
TETEFIAWGPTGDED
SEQ ID NO: 16:
EDDDWIDWYDSDDD
SEQ ID NO: 17:
EDDDWIDWGPTSDDD
SEQ ID NO: 18:
EDDYIDWYDSDDD
SEQ ID NO: 19:
EDDYIDWGPTSDDD
SEQ ID NO: 20:
EDDD(2Nala)IDWYDSDDD
SEQ ID NO: 21:
EDDD(2Nala)IDWGPTSDDD
SEQ ID NO: 22:
EDDYTDWYDSDDD
SEQ ID NO: 23:
EDDYTDWGPTSDDD
SEQ ID NO: 24:
ETEFIAWGPTADED
SEQ ID NO: 25:
ETEYIAWGPTGDED
SEQ ID NO: 26:
ETEFIDWGPTGDED
SEQ ID NO: 27:
EDDSWIWWGPTGDED
SEQ ID NO: 28:
EDDSWIWWYGADTD
SEQ ID NO: 29:
EDDSWIDWGPTGDED
SEQ ID NO: 30:
EDDSWIDWYGADTD
SEQ ID NO: 31:
ETEFI(Cya)WGPTGDED
SEQ ID NO: 32:
EDDD(Oetyr)IDWYDSDDD
SEQ ID NO: 33:
EDDD(Obtyr)IDWYDSDDD
SEQ ID NO: 34:
EDDD(Oetyr)IDWGPTSDDD
SEQ ID NO: 35:
EDDD(Obtyr)IDWGPTSDDD

or a functional variant thereof, wherein the functional variant comprises 1 or 2 individual amino acid substitutions.
8. The peptide according to anyone of the preceding items, wherein the peptide is N-terminally acetylated (Ac—N-ter) and/or C-terminally amidated (—NH₂).
9. The peptide according to anyone of the preceding items, wherein the peptide is conjugated to a cell-penetrating peptides (CPP), such as wherein the CPP is conjugated to the N-terminus or to the C-terminus of the peptide, optionally via a linker, such as via a peptidic linker.
10. The peptide according to anyone of the preceding items, wherein the CPP is

Tat,
(SEQ ID NO: 55)
YGRKKRRQRRR,
R8,
(SEQ ID NO: 56)
RRRRRRRR,
R7,
(SEQ ID NO: 57)
RRRRRRR,
R6,
(SEQ ID NO: 58)
RRRRRR,
R5,
(SEQ ID NO: 59)
RRRRR,
R4,
(SEQ ID NO: 60)
RRRR,
R3,
RRR,
or
R2,
RR.

11. The peptide according to anyone of the preceding items, wherein the peptide is selected from the group consisting of:

SEQ ID NO: 15:
Ac-TETEFIAWGPTGDED-NH₂
SEQ ID NO: 16:
Ac-EDDDWIDWYDSDDD-NH₂
SEQ ID NO: 17:
Ac-EDDDWIDWGPTSDDD-NH₂
SEQ ID NO: 18:
Ac-EDDYIDWYDSDDD-NH₂
SEQ ID NO: 19:
Ac-EDDYIDWGPTSDDD-NH₂
SEQ ID NO: 20:
Ac-EDDD(2Nala)IDWYDSDDD-NH₂
SEQ ID NO: 21:
Ac-EDDD(2Nala)IDWGPTSDDD-NH₂
SEQ ID NO: 22:
Ac-EDDYTDWYDSDDD-NH₂
SEQ ID NO: 23:
Ac-EDDYTDWGPTSDDD-NH₂
SEQ ID NO: 24:
Ac-ETEFIAWGPTADED-NH₂
SEQ ID NO: 25:
Ac-ETEYIAWGPTGDED-NH₂
SEQ ID NO: 26:
Ac-ETEFIDWGPTGDED-NH₂
SEQ ID NO: 27:
Ac-EDDSWIWWGPTGDED-NH₂
SEQ ID NO: 28:
Ac-EDDSWIWWYGADTD-NH₂
SEQ ID NO: 29:
Ac-EDDSWIDWGPTGDED-NH₂
SEQ ID NO: 30:
Ac-EDDSWIDWYGADTD-NH₂
SEQ ID NO: 31:
Ac-ETEFI(Cya)WGPTGDED-NH₂
SEQ ID NO: 32:
Ac-EDDD(Oetyr)IDWYDSDDD-NH₂
SEQ ID NO: 33:
Ac-EDDD(Obtyr)IDWYDSDDD-NH₂
SEQ ID NO: 34:
Ac-EDDD(Oetyr) IDWGPTSDDD-NH₂
SEQ ID NO: 35:
Ac-EDDD(Obtyr)IDWGPTSDDD-NH₂
SEQ ID NO: 36:
Ac-RRRRRRRREDDDWIDWYDSDDD-NH₂
SEQ ID NO: 37:
Ac-RRRREDDDWIDWYDSDDD-NH₂
SEQ ID NO: 38:
Ac-RRREDDDWIDWYDSDDD-NH₂
SEQ ID NO: 39:
Ac-RRRRRRRREDDDWIDWGPTSDDD-NH₂
SEQ ID NO: 40:
Ac-RRRREDDDWIDWGPTSDDD-NH₂
SEQ ID NO: 41:
Ac-RRREDDDWIDWGPTSDDD-NH₂
SEQ ID NO: 42:
Ac-RRRREDDD(2Nala)IDWYDSDDD-NH₂
SEQ ID NO: 43:
Ac-RRRREDDD(2Nala)IDWGPTSDDD-NH₂

or a functional variant thereof, wherein the functional variant comprises 1 or 2 individual amino acid substitutions.
12. The peptide according to anyone of the preceding items, wherein the peptide has binding affinity (Kᵢ) to a sushi domain 1/2 (SD1/2) protein of 20 nM or less; and/or
   wherein the peptide has membrane permeability (CP₅₀) of 10 μM or less; and/or
   wherein the peptide has plasma stability (T_{1/2}) of 12 hours or more.
13. The peptide according to anyone of the preceding items, wherein the peptide is capable of inhibiting an interaction between a protein and a SD1/2 of GB1a at presynaptic neurons; and/or
   wherein the peptide is capable of inhibiting an interaction between:
   sushi domain 1 (SD1) and adherence-junction associated protein 1 (AJAP1),
   SD1 and PILRα-associated neural protein (PIANP), and/or
   SD1 and amyloid precursor protein (APP).
14. A peptide according to anyone of the preceding items for use as a medicament.
15. A peptide according to anyone of items 1 to 13 for use in the treatment of neurological and/or psychiatric disorders, such as wherein the neurological disorder is epilepsy, spasticity, schizophrenia, dystonia, neuropathic pain, narcolepsy, spasticity, cognitive deficits such as Mild cognitive impairment (MCI), autism spectrum disorder and/or substance-use disorders; and/or wherein the psychiatric disorder is depression, anxiety and/or Attention deficit hyperactivity disorder (ADHD).

### EXAMPLES

### Example 1. General Methods and Material

Except where otherwise stated, amino acids and reagents for SPPS were purchased from Iris Biotech or Gyros Protein Technologies. Reagents and solvents were purchased from commercial suppliers and used without further purification. Synthesized crude peptides were purified using a preparative reverse phase high performance liquid chromatography (RP-HPLC) system (Waters) with a reverse phase C18 column (Zorbax, 300 SB-C18, 21.2×250 mm) with a binary buffer system of H₂O:CH₃CN:TFA (A: 95:5:0.1; B: 5:95:0.1) at 20 mL/min. Mass spectra of peptides were characterized using the electron spray ionization (ESI) liquid chromatography-mass spectrometry (LC-MS) coupled to an Agilent 6410 Triple Quadrupole Mass with a reverse phase C18 column (Zorbax Eclipse XBD-C18, 4.6×50 mm) using a binary buffer system consisting of H₂O:CH₃CN:formic acid (A: 95:5:0.1; B: 5:95:0.1) at 0.75 mL/min. The purity of synthesized peptides were determined by at 214 nm UV absorbance on an analytical reverse phase ultra-performance liquid chromatography (RP-UPLC) (Waters) system with a reverse phase C18 column (Acquity UPLC BEH C18, 1.7 μm 2.1×50 mm) using a binary buffer system consisting of H₂O:CH₃CN:TFA (A: 95:5:0.1; B: 5:95:0.1) at 0.45 mL/min.

SPOT peptide array synthesis. μSPOT peptide arrays (CelluSpots, Intavis AG, Cologne, Germany) were synthesized using a RePepSL synthesizer (Intavis AG) on acid labile, amino functionalized, cellulose membrane discs (Intavis AG) containing 9-fluorenylmethyloxycarbonyl-β-alanine (Fmoc-β-Ala) linkers (minimum loading 1.0 μmol/cm). Synthesis was initiated by Fmoc deprotection using 20% piperidine in N-methylpyrrolidone (NMP) (1×2 and 1×4 μL, 3 and 5 min, respectively) followed by washing with dimethylformamide (DMF, 7×100 μL per disc) and ethanol (EtOH, 3×300 μL per disc).

Peptide chain elongation was achieved using 1.2 μL of coupling solution consisting of preactivated amino acids (AAs, 0.5 M) with ethyl 2-cyano-2-(hydroxyimino)acetate oxyma (1.5 M) and N,N′-diisopropylcarbodiimide (DIC, 1.1 M) in NMP (2:1:1, aa:oxyma:DIC). The couplings were carried out 6 times (10, 15, 25, 30, 30 and 30 min, respectively), and subsequently, the membrane was capped twice with capping mixture (5% acidic anhydride in NMP), followed by washes with DMF (7×100 μL per disc). After chain elongation, final Fmoc deprotection was performed with 20% piperidine in NMP (3×4 μL, 5 min each), followed by 6× washes with DMF, subsequent N-terminal acetylation with capping mixture (3×4 μL, 5 min each) and final washes with DMF (7×100 μL per disc) and EtOH (7×200 μL per disc). Dried cellulose membrane discs were transferred to 96 deep-well blocks and were treated with the side-chain deprotection solution consisting of 80% TFA, 12% DCM, 5% H₂O, and 3% TIPS (150 μL per well) for 1.5 h at room temperature (rt). Afterward, the deprotection solution was removed, and the discs were solubilized overnight at rt using a solvation mixture containing 88.5% TFA, 4% trifluoromethanesulfonic acid (TFMSA), 5% H₂O, and 2.5% TIPS (250 μL per well). The resulting peptide-cellulose conjugates were precipitated with ice-cold ether (1 mL per well) and spun down at 1000 rpm for 90 min, followed by an additional wash of the formed pellet with ice-cold ether. The resulting pellets were redissolved in DMSO (500 μL per well) to give final stocks, which were transferred to a 384-well plate and printed (in duplicates) on white coated CelluSpots blank slides (76×26 mm, Intavis AG) using a SlideSpotter robot (Intavis AG).

Quality control of SPOT. Cleavable peptides as synthesis quality controls were prepared following general SPOT synthesis procedure (see above). For peptide cleavage from the cellulose support Fmoc-Rink amide linker was coupled as a first building block (4 times at 10, 15, 25, and 30 min, respectively), followed by deprotection and standard SPOT peptide chain elongation procedure. The discs with synthesized peptides were transferred into Eppendorf tubes and treated with side chain deprotection solution 80% TFA, 12% DCM, 5% H₂O, and 3% TIPS (150 μL per well) for 1.5 h. Subsequently, the cleaved peptides were precipitated with ice-cold diethyl ether and spun down at 14 000×g (4° C., 20 min). The precipitate was dissolved in 100 μL of 50:50:0.1 (CH₃CN:H₂O:TFA), filtered (Fisherbrand™ Syringe Filters: PTFE Membrane, 0.2 μM) and analyzed by LC-MS and RP-UPLC.

SPOT peptide array screening based on slide imaging method. The DMSO stocks of SPOT synthesized peptide-cellulose conjugates were pipetted into a 384-well plate and then printed in duplicates onto the white coated CelluSpots slides (76×26 mm, Intavis AG) using a SlideSpotter robot (Intavis AG). After air drying overnight, the slides were rinsed gently with 1× phosphate buffered saline (PBS, pH 7.4), and then blocked in blocking solution (5% bovine serum albumin (BSA) in PBS) for >2 h at rt on an orbital shaker. Subsequently, the slides were incubated with 6×His-tagged SD1/2 protein in blocking solution at different concentrations (epitopes mapping: 0.25 μM, 0.01 μM and 0.05 μM for APP, AJAP-1 and PIANP, respectively. Deep mutational scans: 0.02 μM, 0.005 μM and 0.01 μM for APP, AJAP-1 and PIANP, respectively) for 1 h at rt. After briefly rinsing with washing solution (PBS-T: 0.05% Tween-20 in PBS buffer), the slides were washed 3 times by PBS-T for 2 min. Subsequently, slides were incubated with diluted HRP conjugated 6×His Tag antibody (1:10000, ab184607, abcam) for 30 min at rt. Finally, the slides were washed 5×2 min with washing solution and then visualized with SuperSignal West Femto Maximum Sensitivity Substrate (ThermoFisher Scientific) using Syngene P×i image system. The resulting SPOT slide images were analyzed using the Array Analyze Software (Active Motif) to quantify the relative SD1/2 binding of each peptide.

SPOT peptide array screening based on fluorescence polarization assay. FP assay was employed to explore the possibility of using SPOT synthesized peptide-cellulose conjugates to compete the binding between AJAP-1_{probe} (SEQ ID NO: 15) and SD1/2 in solution to generate reproducible dose-response curves in black flat bottom 384-well plates (Corning Life Science).

The DMSO stocks of all positional scan peptide-cellulose conjugates were diluted 10, 320 and 10 times (for APP, AJAP-1, PIANP-derived peptides, respectively) directly into 1×PBS (pH 7.4). All experiments were conducted following standard FP assay protocol by using 25 nM of AJAP-1_{probe} and 40 nM of recombinant SD1/2 protein (Dinamarca et al, 2019) in 100 mM PBS (pH 7.4) containing 1% bovine serum albumin (BSA) at rt. Subsequently, the fresh prepared diluted peptide-cellulose conjugates were pipetted into the flat bottom black 384-well plates (Corning Life Science) incubating with the pre-mixed SDs/AJAP-1_{probe}. The fluorescence polarization values were measured using excitation/emission wavelengths of 530/580 nm with a Safire2 plate reader (Tecan). The instrumental Z-factor was adjusted to obtain the maximum fluorescence intensity and the G-factor was calibrated to give an initial 20 mP (millipolarization value) of the reference wells. The obtained the polarization value (mP) from each well represents the relative binding affinity of the mutated peptide compared to the corresponding wild type peptides.

### Solution Array Synthesis.

Parallel synthesis in 96 format peptide synthesis was done using Intavis multipep RSi instrument. Fmoc-PAL resins was used (10 mg per well). Fmoc-amino acids (Fmoc-AA) were dissolved in dimethylformamide (DMF) containing 0.3 M OxymaPure. Throughout the synthesis Fmoc-AA were preactivated with diisopropylcarbodiimide (DIC) for 2 min before adding to the resin. Three couplings using 100 μL Fmoc-AA and 10 μL DIC (3 M in DMF) pr well were employed with coupling times of 5, 15 and 60 min. Removal of Fmoc was performed by washing twice with 120 μl per well of 25% piperidine in DMF for 2 and 8 min. Washing steps after deprotection with piperidine and after coupling steps included washing with DMF (150 μL per well) five times. After synthesis the resins were dried with ethanol and trifluoroacetic acid (TFA) was added using 92% TFA containing 4% triisopropylsilane (TIPS), 1% thioanisol, and 3% H₂O for 2 hours. This was done by adding 100 μL per well of the TFA cleavage cocktail over a period of 30 min adding up to a total volume of 1 mL to each well. TFA was then reduced in volume to approximately 100-150 μL by N₂ flow and peptides were precipitated by the addition 800 μL of diethylether to each well. The peptides were transferred to Waters solvinert plates and washed thoroughly five times with diethylether 500 μL per well. After washing, the peptides were dried and then dissolved in 80% DMSO. Characterization was performed by UPLC-MS on a setup consisting of a Waters Acquity UPLC system connected to an LCT Premier XE mass spectrometer from Micromass using solvent A 0.1% TFA in H₂O and solvent B 0.1% TFA in CH₃CN. The peptides were confirmed by MS and had an average purity of >80%.

Peptide synthesis. All peptides were synthesized by solid-phase peptide synthesis (SPPS) using the Fmoc-strategy on the H-Rink amide ChemMatrix resin (Sigma-Aldrich) ranging from 0.01 to 0.1 mmol scale. Standard amide condensation reactions were carried out at rt under agitation using the coupling mixture with the equivalent ratio of 1:4:4:8 (resin:protected AAs:HCTU:DIPEA) and were monitored using a Kaiser Test Kit (Sigma-Aldrich). Fmoc deprotection was performed 2 times and 2 min for each using 20% piperidine in DMF and followed by extensive washing with DMF. After completion of elongation, the N-terminal Fmoc was deprotected by 20% piperidine and then capped for 5 min using 5% acid anhydride in DMF at the presence of 8 equivalents of DIPEA and then the resin was washed with DMF and DCM before being dried under vacuum.

Automated peptide synthesis. Automated peptide synthesis using Fmoc-based SPPS was carried out either on a Prelude X or PurePep Chorus peptide synthesizers (Gyros Protein Technologies, Tucson, AZ, USA) with 10 mL (0.05 mmol scale) or 25 mL (0.1 mmol scale) glass reaction vessels using H-Rink amide ChemMatrix resin (Sigma-Aldrich).

For Prelude X, all reagents were prepared as DMF solutions: Fmoc-protected amino acids (0.2 M), HCTU (0.4 M), and DIPEA (1.0 M). Sequences were elongated as the following procedures: deprotection (2×2 min, rt, 300 rpm shaking) and coupling (2×5 min, 65° C., 300 rpm shaking, for Cys and His couplings 2×5 min, 50° C., 300 rpm shaking). Amino acids were double coupled using amino acid/HCTU/DIPEA (ratio=1:1:2.5) in 5-fold equivalent excess over the resin scale to ensure synthetic efficiency. For PurePep Chorus, all reagents were also prepared as solutions in DMF: Fmoc-protected amino acids (0.1 M), Oxyma Pure (0.4 M) and DIC (0.4 M). Sequences were elongated as the following procedures: deprotection (2×2 min, rt, 350 rpm shaking) and coupling (2×7 min, 65° C., 350 rpm shaking, for Cys and His couplings 2×7 min, 50° C., 350 rpm shaking). Amino acids were double coupled using amino acids/DIC/Oxyma pure (ratio=1:1.05:1.1) in 4-fold equivalent excess over the resin scale to ensure synthetic efficiency.

TAMRA labelling. For FP assay, the fluorescent peptides were synthesized on resin by coupling the fluorophore TAMRA (Anaspec Inc.) onto the free N-terminus of peptides. The reaction was performed at rt using a mixture of TAMRA/PyBOP/DIPEA (ratio=1.5:1.5:3) in NMP. For minimizing photobleaching, the agitated reaction vessels were covered by tinfoil. After about 16 h, the resin was washed extensive with DMF and DCM followed by drying under vacuum before the final cleavage.

Peptide cleavage and purification. The synthesized peptides were cleaved from the resin using the cleavage cocktail containing 92.5% TFA, 2.5% H₂O, 2.5% TIPS and 2.5% DODT for 2 h under agitation at rt. After cleavage, the peptides were precipitated with ice-cold diethyl ether and centrifuged at 4350 rpm for 10 min at 4° C. The resulting crude peptides were redissolved in 50:50:0.1 (H₂O:CH₃CN:TFA), analyzed using LC-MS and UPLC and lyophilized for purification (as described in General methods section). The collected fractions from purification were characterized by LC-MS. The purity of the fractions was determined at 214 nm using UPLC. The fractions with higher than 95% purity were pooled and lyophilized for further experiments.

Fluorescence polarization (FP). FP assay was employed to determine the binding affinity of peptides in flat bottom black 384-well plates (Corning Life Science) using a Safire2 plate reader (Tecan). All experiments were conducted in 1×PBS (pH 7.4) containing 1% bovine serum albumin (BSA) at rt. The fluorescence polarization values were measured using excitation/emission wavelengths of 530/580 nm. The instrumental Z-factor was adjusted to obtain the maximum fluorescence intensity and the G-factor was calibrated to give an initial millipolarization value of 20 mP. All FP assays including saturation or competition experiments were performed using 25 nM of TAMRA labeled peptides as probes. For saturation assay, the millipolarization values were measured after mixing of probes with 12 serial 1 to 1 diluted concentrations of Sushi domains (SD1/2) protein. All experiments were performed in triplicate and the data were plotted to the one-site binding model using GraphPad Prism 9 to calculate the K_{d} of probes. In the competition assay, pre-mixed of SDs/probes were outcompeted with unlabeled peptides at 12 serial 1 to 1 diluted concentrations ranging from 0.24 to 500 μM based on the expected affinity. The experiments were performed at least in triplicate and the data were fitted to the sigmoidal dose response curve using GraphPad Prism 9 to obtain the IC₅₀ of peptides. The Kᵢ values were calculated according to Nikolovska-Coleska et al³¹⁰.

### Results

Taken together, SPOT identified several peptide hits from each APP, AJAP-1 and PIANP (data not shown), which were combined to three 21-mer length SDs binding epitopes (APP₁₉₇₋₂₁₇, AJAP-1₁₇₃₋₁₉₃ and PIANP₉₃₋₁₁₃). Moreover, based on SPOT results the overlapping 9 residues length regions (²⁰³EDDSDVWWG²¹¹ SEQ ID NO: 4, ¹⁷⁹EFIAWGPTG¹⁸⁷ SEQ ID NO: 5, and ⁹⁹AIVWGPTVS¹⁰⁷ SEQ ID NO: 6) within APP, AJAP-1 and PIANP, respectively, were indicated representing the core binding regions to SD1/2. Additionally, alignment of all peptide hits to the previously reported two critical residues (WG) within the three proteins, the overlapped 5 amino acids, ²⁰⁷DVWWG²¹¹ SEQ ID NO: 7, ¹⁷⁹FIAWG¹⁸⁷ SEQ ID NO: 8 and ⁹⁹AIVWG¹⁰³ SEQ ID NO: 9 within APP, AJAP-1 and PIANP, respectively, were anticipated as the most essential residues for SD1/2 binding and highlighted in the red dashed frame.

High-affinity AJAP-1_{probe} facilitated determination of binding constants of peptides 11 (SEQ ID NO: 11) and 12 (SEQ ID NO: 12) (Kᵢ=5 and 1.3 μM, respectively). Thereafter, we successfully characterized the binding affinities of all synthesized SPOT hits by FP using the AJAP-1_{probe} (data not shown). AJAP-1 derived peptides exhibited the highest SD1/2 binding affinity with Kᵢ values all below 100 nM. Additionally, peptide 15 (SEQ ID NO: 15) again proved to be the highest binding affinity hit (Kᵢ=23 nM). In contrast, peptides derived from the other two proteins exhibited much lower binding affinity, with peptides 13 (EDDSDVWWGGADTDY, SEQ ID NO: 13) (581 nM) and 14 (PWAIVWGPTVSREDG, SEQ ID NO: 14) (465 nM) being the best SPOT-identified hits from APP and PIANP, respectively (Table 3).

### Example 2. Definition of the Optimal Binding Motif

We first selected three peptides (peptides 15 (SEQ ID NO: 15), 36 (SEQ ID NO: 15) and 14 (SEQ ID NO: 14), displaying the highest binding affinity towards SD1/2, as a starting point for definition of optimal and the shortest possible binding interface. Thus, we next focused on systematic truncation scan at both N- and C-terminus of all three SD-binding peptides.

### Results

The information form elongation and truncation scans (Tables 4, 5 and 6) was used to design, synthesize and evaluate, the shortest possible 14-mer binding epitopes-APP_{WT} (Ac-EDDSDVWWGGADTD-NH₂ (SEQ ID NO: 1)), AJAP-1_{WT} (Ac-ETEFIAWGPTGDED-NH₂ (SEQ ID NO: 2)) and PIANP_{WT} (Ac-PWAIVWGPTVSRED-NH₂ (SEQ ID NO: 3)). Calculated Kᵢ values from FP experiments suggested a rank order of SD1/2 binding affinities—AJAP-1 (31±1 nM)>>PIANP (476±19 nM)>APP (557±9 nM). Alignment of the three binding regions to the WG (positions 0 and 1, respectively) motif revealed overall similar structure consisting of a hydrophobic core (positions-3 to 2) surrounded by polar residues at both termini, apart from PIANP_{WT} containing a more hydrophobic N-terminal.

### Example 3. Alanine-Scan, D-Amino Acid Scan and N-Methylation Scan

An Ala scan on the identified APP_{WT}, AJAP-1_{WT} and PIANP_{WT} was performed to elucidate the importance of each residue for the molecular recognition. 39 peptide variants (13 from each binding motif) each containing a single alanine substitution were synthesized and subsequently evaluated in the FP assay for their binding to SD1/2.

Similarly, a D-amino acid scan and N-methylation scan were also conducted.

### Results

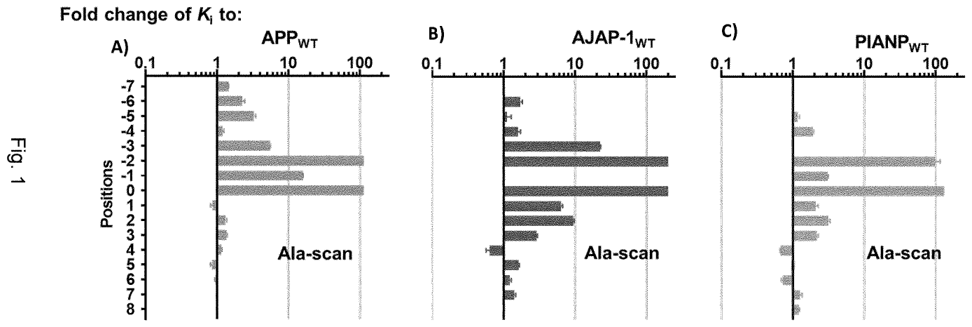

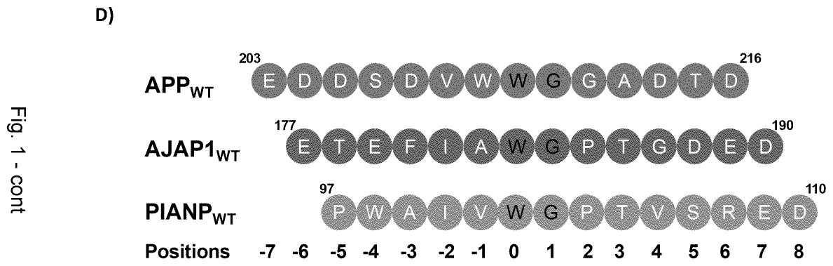

Comparison of the resulting binding affinities indicated similar tendencies as seen in truncation scan, underlining the importance of hydrophobic core (FIG. 1). More specifically, the most critical residues were identified within the core at positions-2 and 0 (corresponding to positions 6 and 8 in the peptide consensus of claim 1, SEQ ID NO: 64) and substitutions to Ala resulted in ≥100-fold, whereas the Ala mutations at the remaining positions showed to affect the affinity to lesser extent. The major differences between different peptides were seen when substituting residues at positions 1-3. APP_{WT} peptide Ala variants at these positions showed almost no changes in binding affinity, while AJAP-1_{WT} and PIANP_{WT} Ala scan peptides exhibited 2-10-fold lower binding affinity to SD1/2.

Similarly, D-amino acids scan revealed that the stereochemistry of the hydrophobic residues in the core (positions −2, −1 and 0, corresponding to positions 6 to 8 in the peptide consensus of claim 1, SEQ ID NO: 64) of peptides are critical for SD1/2 binding, resulting in peptides variants with 100-200-fold affinity decrease. Additionally, at none of the positions, incorporation of a D-amino acid resulted in an enhanced binding affinity of SD1/2.

Similarly, to the previous scans, amino acid substitutions with the corresponding N-Me amino acids again highlighted the importance of the core binding region. Specifically, N-methylation at positions −1 and 1 (corresponding to positions 7 and 9 in the peptide consensus of claim 1, SEQ ID NO: 64) exhibited the largest impact negative impact on the peptides binding to SD1/2 compared to the parent peptide variants. Interestingly, substitutions of position 2 amino acid (corresponding to position 10 in the peptide consensus of claim 1, SEQ ID NO: 64) with N-Me-Gly at APP_{WT} yielded a peptide variant showing 3-fold increased affinity. Both AJAP-1_{WT} and PIANP_{WT} peptides at position 2 (corresponding to position 10 in the peptide consensus of claim 1, SEQ ID NO: 64) have a Pro residue, which provides rigidity. N-Me-Gly substitution introduced in APP_{WT} likewise introduce conformational constrains, thus potentially leading to preferred binding conformation and increased binding affinity.

The binding affinity for all synthesised and tested peptides are shown in Tables 7, 8 and 9.

### Example 4. Deep Mutational Scan of APP_{WT}, AJAP-1_{WT} and PIANP_{WT} Peptides

We applied the deep mutational scanning method using canonical amino acids (cAAs) for peptides of APP_{WT}, AJAP-1_{WT} and PIANP_{WT}. To achieve this, we again employed SPOT technology and generated three arrays and each comprising 280 14-mer peptides (including 14 wild peptides as references).

We synthesized additional positional scanning array (termed solution array) of APP_{WT} using the conventional on-resin SPPS strategy in 96-well plates. This solution array contained 268 peptides (including two wild type peptides). Noteworthy, all peptides synthesized in this array showed around 90% crude purity. Taking advantage of the high purity of this peptide array, we generated six-point FP inhibition curve of each peptide and the obtained dose response curves enabled us to calculate the binding affinity (Kᵢ) of each peptide variant (data not shown).

### Results

The obtained Kᵢ (635 nM) using the crude APP_{WT} (Table 10) in the solution array is almost identical to the previously measured Kᵢ (557 nM) using the purified APP_{WT}. The solution array results revealed dramatically increased affinity of the two most interesting mutants of APP_{WT}, D207W and G211Y, which showed Kᵢ values of 78 nM and 167 nM, respectively. Substitution of D207 with bulkier hydrophobic amino acids including Phe, Tyr and Trp enabled additional contacts with hydrophobic residues at SD1.

FP-based screen of SPOT synthesized AJAP-1_{WT} (Table 11) positional scan array identified 7 residues within AJAP-1_{WT}, ¹⁷⁹EFIAWGP¹⁸⁵ (SEQ ID NO: 62), as the highly conserved core binding motif, which was consistent with the SPOT epitope mapping experiments. Multiple substitutions of the A182, located between two critical residues (I181 and W183), generated mutants exhibiting comparable or even higher binding affinity compared to AJAP-1_{WT}. Particularly, the mutant A182D (peptide 26, SEQ ID NO: 26) represented the most promising mutation of AJAP-1_{WT} with dramatically increased binding affinity (Ki=9 nM).

Two Arg residues are present in SD1 (R32 and R36) are in proximity to the A182. Therefore, A182D mutation might facilitate additional electrostatic interaction between these thereby resulting in improved binding affinity.

T178 could be mutated into hydrophobic amino acids such as Ile and Val with improved affinity. Furthermore, F180Y, showed a potential to improve affinity.

Deep mutational scanning of PIANP_{WT} (Table 12) revealed a highly conserved hydrophobic core binding motif composed of 6 residues (¹⁰⁰IVWGPT¹⁰⁵ (SEQ ID NO: 63)). Identified beneficial mutations of PIANP_{WT} were relating mostly to mutations of hydrophobic residues into acidic amino acids. However, just slight affinity improvements (less than 2-fold) compared to the PIANP_{WT} were observed.

### Example 5. NcAA Deep Mutation Scan of APP_{WT}, AJAP-1_{WT} and PIANP_{WT} Peptides

We performed computational ncAAs based deep mutational scanning of APP_{WT}, AJAP-1_{WT} and PIANP_{WT} using the generated random forest (RF) models to predict the relative SD1/2 binding affinity of each mutant with introduced single ncAA. Specifically, each residue within peptides was individually replaced by any of the available 67 ncAAs in the z-scales database, resulting in 938 variants having a single ncAA introduced in each wild type peptide.

### Results

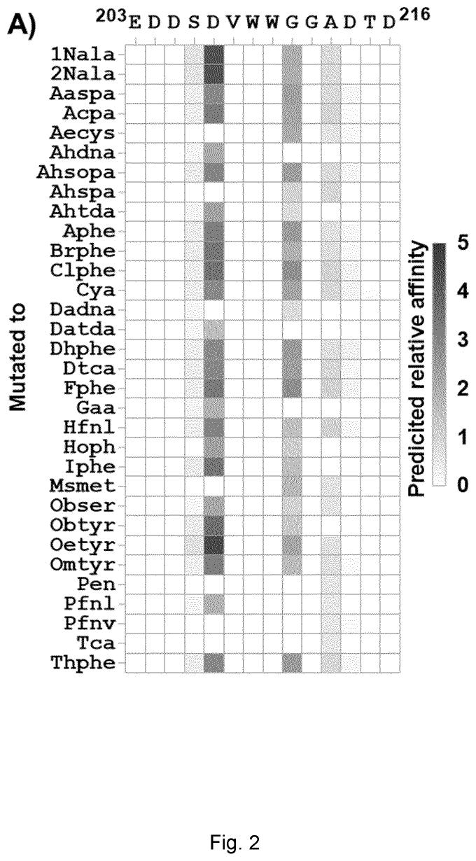

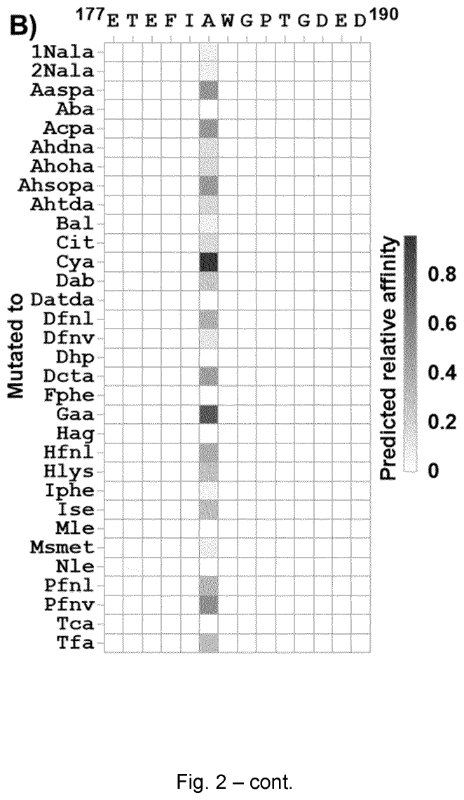

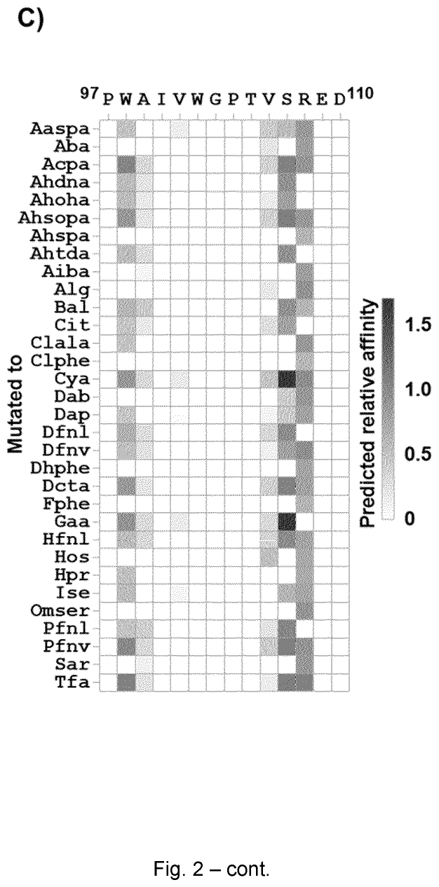

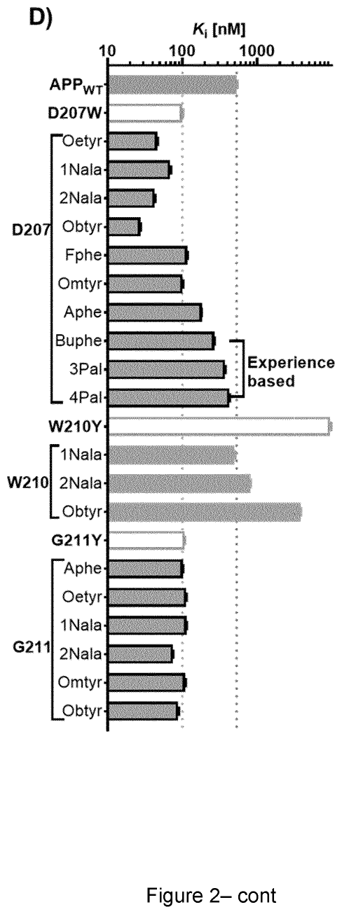

The beneficial ncAA mutations for APP_{WT} were mainly related to mutation of residues Asp207 and Gly211 into aromatic and/or hydrophobic ncAAs. The relative affinities of peptides with ncAAs were organized as three individual heat maps (FIG. 2). Based on the relative affinity, we selected and synthesized 17 peptides containing ncAAs and tested their binding affinity towards SD1/2 (Table 16). Overall, the computational prediction of the relative affinity showed a good correlation with the FP affinity data. Notably, all 7 mutations of the residue Asp207 within APP_{WT} exhibited improved affinity (2- to 19-fold) compared to APP_{WT} and 4 mutants exhibited even higher affinity (~2- to 4-fold) than the best cAA mutant Asp207Trp (Kᵢ=100 nM). Substitutions of Asp207 with 3-(2-naphthyl)-L-alanine (2-Nala) (Kᵢ=43 nM), O-benzyl-L-tyrosine (Obtyr) (Kᵢ=27.1 nM) and O-ethyl-L-tyrosine (Oetyr) (Kᵢ=46.3 nM) displayed 2-, 4- and 2-fold increased affinity compared to Asp207Trp, respectively. Furthermore, all 6 mutations of Gly211 also displayed greatly improved affinity (around 100 nM) compared to the wild type. For AJAP-1_{WT}, the Ala182Cya (Kᵢ=11.8 nM) mutation showed similar affinity as Ala182Asp (Kᵢ=9), which represented the best identified cAAs mutant of AJAP-1_{WT}, exhibiting 3-fold improved affinity relative to the wild type.

### Example 6. Affinity Maturation

Design of novel SD-specific high affinity peptides based on APP_{WT} and AJAP-1_{WT}. We first divided both wild type peptides into the N-terminal and C-terminal parts, separating them at positions 0 and 1. Then for the design of novel peptides one part (either N- or C-terminal) was taken from APP_{WT} and other part (either N- or C-terminal) from AJAP-1_{WT}, generating two hybrid peptides X (Ac-EDDSDVWWGPTGDED-NH₂ SEQ ID NO: 48) and Y (Ac-ETEFIAWGGADTD-NH₂ SEQ ID NO: 49). The hybrid peptide X was obtained by merging APP_{WT} N-terminal part and AJAP-1_{WT} C-terminal part and displayed similar affinity as AJAP-1_{WT} that is almost 10-fold higher than APP_{WT}. In contrast, the hybrid peptide Y containing AJAP-1_{WT} N-terminal part and C-terminal part of APP_{WT} exhibited comparable affinity as APP_{WT} (Table 1). Next, to further optimize binding properties we introduced the best C-terminal mutation G211Y of APP_{WT} into peptide Y generating a chameleon peptide 37 (Ac-ETEFIAWYGADTD-NH₂ SEQ ID NO: 50), which showed improved affinity comparable to peptides X and AJAP-1_{WT} (Table 2). Then, we substituted the Pro in the AJAP-1_{WT}, generating peptide 38 (Ac-ETEFIAWG (N-Me-G)TGDED-NH₂ SEQ ID NO: 51), which exhibited the same affinity as AJAP-1_{WT} indicating that the N-Me-Gly can mimic peptide binding conformation introduced by Pro (Table 1). After exploring contributions of single modifications, we moved forward with incorporation of multiple beneficial mutations into native or hybrid peptide variants. First, we investigated the effect of introducing double modification, namely D207W and V2081 into peptide X.

This led to peptide 27 (Ac-EDDSWIWWGPTGDED-NH₂ SEQ ID NO: 27) showing 3.8-fold better affinity than 9. With this information we combined D207W and V2081 with G211Y mutations and incorporated them to APP_{WT}, resulting peptide 28 (Ac-EDDSWIWWYGADTD-NH₂ SEQ ID NO: 28) displayed Kᵢ value almost identical to peptide X (Table 1). Encouraged by synergistic effect of multiple mutations on the peptides binding affinity we in introduced the best mutation (A182D) of AJAP-1_{WT} into peptides 27 and 28 at the corresponding positions, generating peptides 29 and 30 with further improved affinity (Table 1). Additionally, we introduced other identified favourable mutations of APP_{WT} including S206D, G212D, T215D and A213S into peptide 30. Additionally, affinity improving mutations (G187S and E189D) from AJAP-1_{WT} SAR study were introduced into the AJAP-1_{WT} derived C-terminal part of peptide 29, together with S206D. Satisfyingly, all incorporated mutations into peptides APP_{WT} and X were compatible and generated peptides 16 (Ac-EDDDWIDWYDSDDD-NH₂ SEQ ID NO: 16) and 17 (Ac-EDDDWIDWGPTSDDD-NH₂ SEQ ID NO: 17) with low nanomolar affinity. Similarly, optimized versions of AJAP-1_{WT} and peptide Y, resulted in two high affinity peptides 18 (Ac-EDDYIDWGPTSDDD-NH₂ SEQ ID NO: 18) and 19 (Ac-EDDYIDWYDSDDD-NH₂ SEQ ID NO: 19) (Table 1).

The 3 the most promising hydrophobic ncAAs were Obtyr, 2Nala and Oetyr, which showed the greatest improvement to the affinity of APP_{WT} by substituting the D207 residue. We incorporated these ncAAs into corresponding positions within peptides 16 and 17. The FP data showed that all but one of resulting peptides (peptides 32-35, SEQ ID NOs: 36 to 39) exhibited slightly higher affinity compared to peptides 16 and 17. In contrast, introduction of the 2Nala into peptide 16 further increased affinity to 1.5 nM (peptide 20). (Table 1). The full list of the synthesised and tested peptides for affinity maturation can be found in Table 13.

TABLE 1
Overview of synthesized peptides for affinity
maturation. Data are displayed as calculated Kᵢ
values (mean ± SEM, n ≥ 3). All peptides have N-
terminal acetylation and C-terminal amidation.

| Peptide | Sequences | Kᵢ (nM) |
|---|---|---|
| APP_{WT} | EDDSDVWWGGADTD | 557 ± 9 |
| X | EDDSDVWWGPTGDED | 62 ± 1 |
| Y | ETEFIAWGGADTD | 354 ± 9 |
| AJAP-1_{WT} | ETEFIAWGPTGDED | 31 ± 1 |
| 37 | ETEFIAWYGADTD | 95 ± 5 |
| 38 | ETEFIAWG(N-Me-G)TGDED | 32 ± 2 |
| 27 | EDDSWIWWGPTGDED | 16 ± 1 |
| 28 | EDDSWIWWYGADTD | 18 ± 1 |
| 29 | EDDSWIDWGPTGDED | 5.6 ± 0.7 |
| 30 | EDDSWIDWYGADTD | 11 ± 0.1 |
| 16 | EDDDWIDWYDSDDD | 3.2 ± 0.2 |
| 17 | EDDDWIDWGPTSDDD | 4.0 ± 0.3 |
| 18 | EDDYIDWYDSDDD | 4.6 ± 0.3 |
| 19 | EDDYIDWGPTSDDD | 3.8 ± 0.2 |
| 20 | EDDD(2Nala)IDWYDSDDD | 1.5 ± 0.2 |
| Scr-20 | (2Nala)EDSDDYDDIDDDW | 31.000 ± 1.000 |
| 33 | EDDD(Obtyr)IDWYDSDDD | 9.3 ± 0.2 |
| 32 | EDDD(Oetyr)IDWYDSDDD | 5.3 ± 0.2 |
| 21 | EDDD(2Nala)IDWGPTSDDD | 5.3 ± 0.2 |
| 35 | EDDD(Obtyr)IDWGPTSDDD | 9.5 ± 0.3 |
| 34 | EDDD(Oetyr)IDWGPTSDDD | 6.5 ± 0.4 |

### Example 7 Pull-Down Experiments

The aim of these experiments was to examine whether the most potent peptide 20 could bind to native SD and examine the selectivity towards the SD.

Covalent immobilization of peptides. Peptides AJAP-1_{WT}, 17 and 20 comprising a C-terminal Cys and a PEG2 linker, were loaded covalently to Dynabeads M-270 Epoxy beads (Thermo Fischer Scientific) according to the protocol of the manufacturer. 25 μL DMSO stocks of peptides (10 mM) were diluted 15 times into 350 μL coupling buffer (100 mM PBS buffer containing 2 M (NH₄)SO₄ and 10 mM TCEP, PH=7.4) and agitated overnight at 37° C. with Dynabeads M-270 Epoxy beads (5 mg, ~3.3×10⁸ beads), which had been washed and equilibrated with coupling buffer before. After removing excess peptides, the Dynabeads M-270 Epoxy beads were washed three times and equilibrated with 100 mM PBS buffer, pH=7.4 and stored at 4° C.

Preparation of whole-brain lysates. Dissected adult mouse brains were placed in ice-cold homogenization buffer (320 mM sucrose, 4 mM HEPES, PH 7.5, 1 mM EDTA, 1 mM EGTA, supplemented with complete EDTA-free protease inhibitor mixture (Roche), 1 mL per 100 mg of brain) and homogenized with a glass-Teflon homogenizer (40 passes) on ice. The homogenized material was cleared by centrifugation at 500×g (4° C., 15 min). The supernatant was carefully removed and ultracentrifuged at 48 000×g (4° C., 45 min) to isolate membrane-enriched fraction (pallet), which was solubilized for 3 h at 4° C. in NETN buffer (100 mM NaCl, 1 mM EDTA, 0.5% Nonidet P-40, 20 mM Tris-HCl, pH 7.4, supplemented with a protease inhibitor mixture). The solubilized membrane-enriched fraction was cleared by ultracentrifugation at 100 000×g (4° C., 45 min). The final brain lysates (at 2 mg/mL protein concentration) were directly used for immunoblot analysis (input lanes) or for pull-down experiments.

Pull-down experiments. The covalently immobilized peptides AJAP-1_{WT}, 16 and 20 were washed with 3×1 mL of 100 mM PBS and 1 mL of 100 mM PBS containing 0.1% Tween-20 and directly incubated with freshly prepared whole-brain lysate (2 mg/ml of total protein) overnight on a spring wheel at 4° C. The next day, the beads were washed with 2×1 mL of 100 mM PBS and 2×1 mL of 100 mM PBS containing 0.1% Tween-20, and then the pulled down proteins were eluted with 4× sample loading buffer containing 200 mM DTT (2×10 μL). Proteins were resolved using standard one-dimensional SDS-PAGE on 10% polyacrylamide gels (for 45 min at 70 mV, followed by an additional 1.5 h at 120 mV). For immunoblotting analysis, proteins were transferred to nitrocellulose membrane (0.2-μm pore size) (iBlot™ 2 Transfer Stacks, nitrocellulose, mini) using iBlot2 (invitrogen) and then probed with the primary antibodies mouse anti-SD clone 43N12 (1:250) or mouse monoclonal to GABA_{B1} subunit (abcam, 1:500) in combination with secondary peroxidase-coupled sheep anti-mouse IgG antibody (Cytiva, 1:5000).

### Results

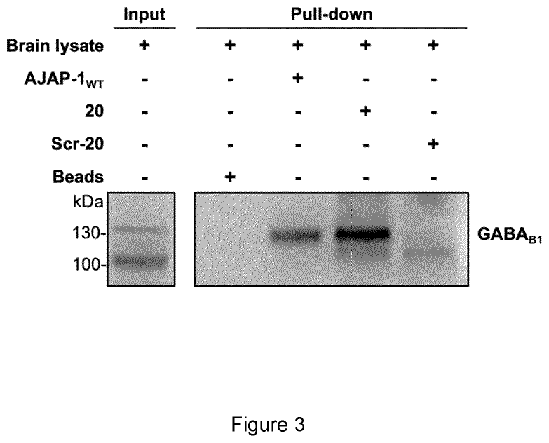

The pull-down experiments demonstrated that both AJAP-1_{WT} and peptide 20 showed the ability to bind and enrich full-length GB1a subunit in a complex native environment, whereas the negative control peptide Scr-20 and beads alone did not interact with the GB1a subunit (FIG. 3).

### Example 8. In Vitro Plasma Stability

For time-course assay, 360 μL human plasma was pre-warmed up at 37° C. for 15 min and then spiked with 40 μL of tested peptides (1 mM in 1×PBS). After 0, 2, 6, 12, 24 and 48 h incubation at 37° C., for each time point 45 μL sample was collected and pre-treated with 50 μL 6 M urea for 10 min at 0° C. Subsequently, 100 μL acetonitrile was added into and then samples were incubated overnight at −20° C. After that, all samples were centrifuged at 13,400 rpm for 10 min and the supernatant was filtered and then analyzed by LC-MS and UPLC (Waters). The area under the curve (AUC) was determined and normalized to the value of the first time point. The half-life (T_{1/2}) was obtained by fitting the data to the one-phase decay equation in GraphPad Prism 9 and expressed as the mean of three individual experiments. For end-point assay, 108 μL human plasma was pre-warmed up at 37° C. for 15 min and then spiked with 12 μL of tested peptides (1 mM in 1×PBS). After 0 and 48 h incubation at 37° C., 45 μL sample was collected and then treated as following the same procedure described in time-course assay above. The area under the curve (AUC) of end-point (48 h incubation) was normalized to the value of the first time point and then the stability was expressed as compound remaining (%).

### Results

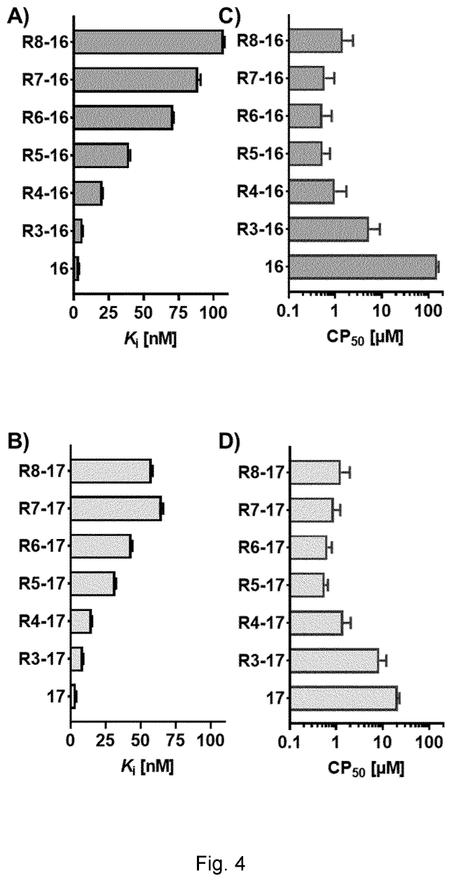

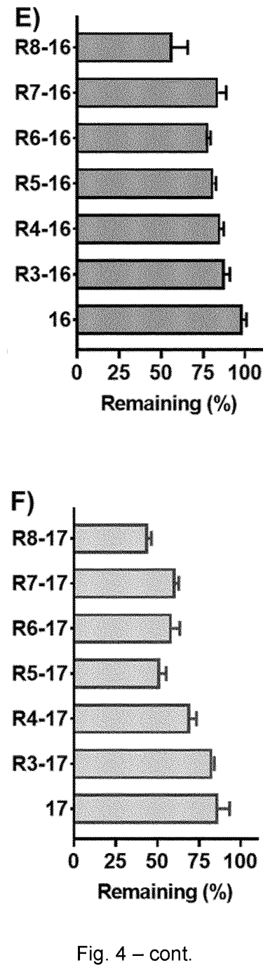

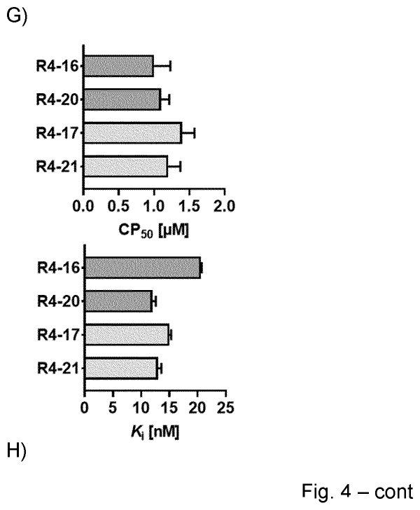

It was found that even the native protein-derived wild type peptides (APP_{WT}, AJAP-1_{WT} and PIANP_{WT}) exhibited excellent stability in human plasma with half-life times of >48, >48 and 16 h, respectively. Similarly, the half life of all four optimized peptides composed of cAAs (16-19) were >48 hours and no apparent degradation was observed after two days incubation with 90% of intact peptide remaining. Moreover, we also investigated the impact of polyR tagging on the plasma stability of peptides. The results demonstrated that R8-tagged peptides (R8-16 and R8-17) showed excellent stability as well with about 50% remaining after 48 hours incubation. Interestingly, truncation of the poly-R from 8R to 3R further improved plasma stability (R3-16 and R3-17), and the amount of the remaining peptide increased from 50% to about 80% after 48 hours incubation (FIG. 4).

Additionally, we evaluated the influence of incorporating the ncAA (2Nala) into 4R tagged version of peptides by characterization of their affinity, permeability, and plasma stability. We found that 4R-20 exhibited about 2-fold higher affinity than 4R-16, while 4R-21 showed almost identical affinity as 4R-17 (FIG. 4). Moreover, the two 4R tagged peptides containing 2Nala (4R-20 and 4R-21) displayed excellent plasma stability (>48 hours half-life) and cell membrane permeability (~1 μM CP50) (FIG. 4 and Table 2).

In conclusion, it was demonstrated that truncating the polyR moieties of our CPP tagged peptides to 4R resulted in excellent membrane permeability, while displaying much less negative impact on the affinity, exhibiting higher human plasma stability compared to 8R tagged peptides.

The full list of the synthesised and tested peptides in combination with a CPP can be found in Table 14.

### Example 9. Chloroalkane Penetration Assay (CAPA)

The HeLa cell line used for CAPA assay was generated by Chenoweth and co-workers and provided by Kritzer and co-workers. The cells stably express a HaloTag-GFP fusion protein anchored through a mitochondria targeting peptide exposed to the cytosol. For CAPA, Hela cells were seeded using DMEM supplemented with FBS in a 96-well plate one day before the experiment at a density of 4×10⁴ cells/well. Just before the assay, the growth media was aspirated off and replaced with Opti-MEM assay media (100 μL/well). DMSO stocks of peptides (10 mM) were diluted in Opti-MEM to prepare serial dilutions of peptides in a separate 96-well PCR plate ensuring the final concentration of DMSO was kept consistent and below 1%. Subsequently, 25 μL of peptide solution was added into each well and the plate was incubated for 4 h at 37° C. with 5% CO₂. After aspirating the assay media off, cells were washed with fresh Opti-MEM (80 μL/well) for 15 min at rt. Following removal of the washing media, cells were incubated with 5 μM CA-TAMRA in Opti-MEM (50 μL/well) for 15 min. Non CATAMRA control wells were incubated with Opti-MEM alone (50 μL/well). After removal of the dye containing media, cells were washed with fresh Opti-MEM (80 μL/well) for 30 min. After aspiration, cells were trypsinized using clear Trypsin (20 μL/well, 10 min at 37° C.), resuspended in PBS containing 2% FBS (180 L/well), and analyzed using a benchtop flow cytometer (Guava EasyCyte, EMD Millipore).

### Results

Due to high similarity, we chose peptides 16 and 17 as models for characterizing and potential optimizing their cell membrane permeability. As expected, CAPA results of CA-tagged peptides 16 and 20 showed only poor cell membrane permeability with CP₅₀ values around 150 μM indicating that it is difficult for highly negatively charged peptides to penetrate cell membranes. Strikingly, the other two similar peptides 17 and 21 showed 2- to 7-fold better membrane permeability compared to peptides 16 and 20, with 17 being the best in permeating the cell membrane (CP₅₀=21 μM) (Table 2).

The full list of the synthesised and tested CA-tagged peptides can be found in Table 15.

TABLE 0
Overview of sythesized CPP tagged peptides. Values of calculated SD1/2
binding affinity (Kᵢ), cell membrane permeability (CP₅₀) and plasma stability
(T_{1/2}) are expressed as mean ± SEM, n ≥ 3.

| | | | CP₅₀ | |
|---|---|---|---|---|
| Peptide | Sequence | Kᵢ (nM) | (μM) | T_{1/2} (h) |
| 16 | EDDDWIDWYDSDDD | 3.2 ± 0.2 | 153 ± 21 | >48 |
| 20 | EDDD(2Nala)IDWYDSDDD | 1.5 ± 0.2 | 146 ± 17 | >48 |
| 17 | EDDDWIDWGPTSDDD | 4.0 ± 0.3 | 21 ± 2 | >48 |
| 21 | EDDD(2Nala)IDWGPTSDDD | 5.3 ± 0.2 | 72 ± 6 | >48 |
| Tat-16 | YGRKKRRQRRREDDDWIDWYDSDDD | 83 ± 1 | 0.8 ± 0.1 | — |
| R8-16 | RRRRRRRREDDDWIDWYDSDDD | 107 ± 1 | 1.5 ± 0.9 | >48 |
| 16-Tat | DDDWIDWYDSDDDYGRKKRRQRRR | 71 ± 1 | 19 ± 0.4 | — |
| 16-R8 | EDDDWIDWYDSDDDRRRRRRRR | 65 ± 1 | 17 ± 0.2 | — |
| R8-RL-16 | RRRRRRRREAAAKEDDDWIDWYDSDDD | 314 ± 4 | — | — |
| R8-FL-16 | RRRRRRRRGGGGSEDDDWIDWYDSDDD | 280 ± 2 | — | — |
| 16-RL-R8 | EDDDWIDWYDSDDDEAAAKRRRRRRRR | 100 ± 4 | — | — |
| 16-FL-R8 | EDDDWIDWYDSDDDGGGGSRRRRRRRR | 118 ± 3 | — | — |
| Tat-17 | YGRKKRRQRRREDDDWIDWGPTSDDD | 50 ± 2 | 2.4 ± 1.0 | — |
| R8-17 | RRRRRRRREDDDWIDWGPTSDDD | 58 ± 3 | 1.3 ± 0.7 | >48 |
| 17-Tat | EDDDWIDWGPTSDDDYGRKKRRQRRR | 79 ± 1 | 6.1 ± 0.8 | — |
| 17-R8 | EDDDWIDWGPTSDDDRRRRRRRR | 123 ± 4 | 17 ± 2 | — |
| R8-RL-17 | RRRRRRRREAAAKEDDDWIDWGPTSDDD | 105 ± 2 | — | — |
| R8-FL-17 | RRRRRRRRGGGGSEDDDWIDWGPTSDDD | 140 ± 4 | — | — |
| 17-RL-R8 | EDDDWIDWGPTSDDDEAAAKRRRRRRRR | 139 ± 2 | — | — |
| 17-FL-R8 | EDDDWIDWGPTSDDDGGGGSRRRRRRRR | 147 ± 9 | — | — |
| R4-20 | RRRREDDD(2Nala)IDWYDSDDD | 12 ± 1 | 1.1 ± 0.2 | >48 |
| R4-21 | RRRREDDD(2Nala)IDWGPTSDDD | 13 ± 1 | 1.2 ± 0.3 | >48 |

To improve cell membrane permeability of the peptides 16 and 17, we decided to conjugate the two prototypical CPPs, Tat (YGRKKRRQRRR SEQ ID NO: 28) and polyR (SEQ ID NOs: 29 to 33) to the N-terminus. The CAPA results demonstrated that both Tat and R8 N-terminally attached to 16 and 17 (R8-16, Tat-16, R8-17 and Tat-17) significantly improved the conjugates permeability with CP₅₀ values around 1 μM (Table 2). However, we found that the positively charged CPPs dramatically influenced the affinity of the negatively charged SD1/2 binding peptides 16 and 17 resulting in 12- to 33-fold loss of affinity.

changing the position of CPPs from the N- to C-terminus had almost no effect of the affinity of the peptides. Moreover, changing in the CPP position resulted in 3- to 23-fold higher CP₅₀ values compared to the corresponding N-terminally tagged peptides (Table 2).

Comparing to CPP-tagged peptides without linkers, adding linkers between CPPs and peptides did not improve binding affinity but in contrast the linker-containing peptides exhibited lower affinity (R8-FL-16/17 and R8-RL-16/17 versus R8-16/17, respectively) (Table 2). The linkers tested were a rigid linker (RL) of sequence EAAAK (SEQ ID NO: 60) and a more flexible linker (FL) of sequence GGGGS (SEQ ID NO: 61).

The CAPA results revealed that truncation to 4R still maintained excellent cell membrane permeability similar to the R8-tagged version with a CP₅₀ around 1 μM. However, further reduction to 3R resulted in substantially decreased permeability (CP₅₀=~10 μM) (FIG. 4).

### Example 10. SRE-Luciferase Accumulation Assay

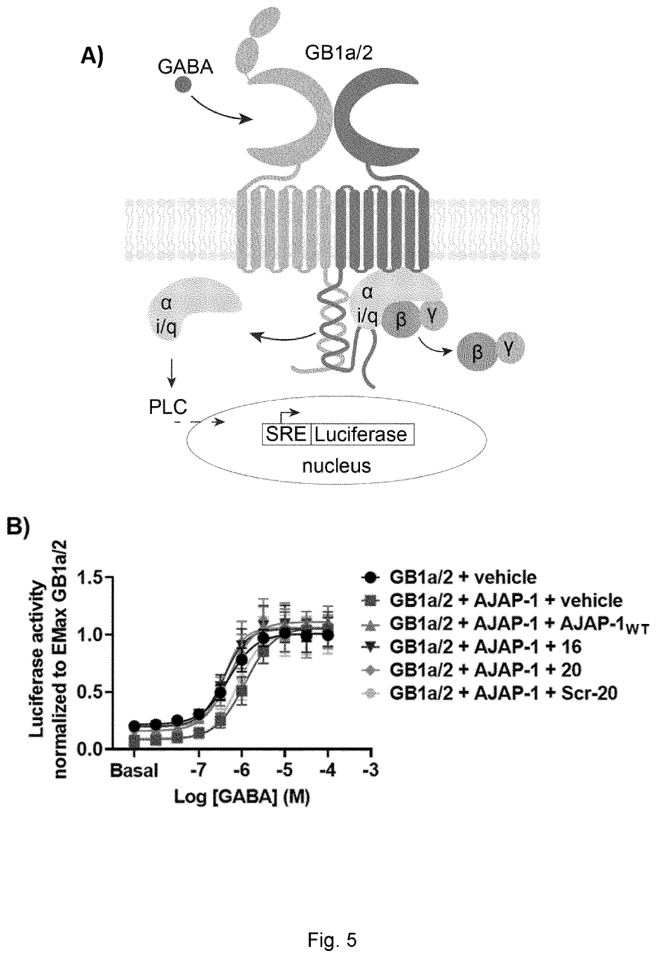

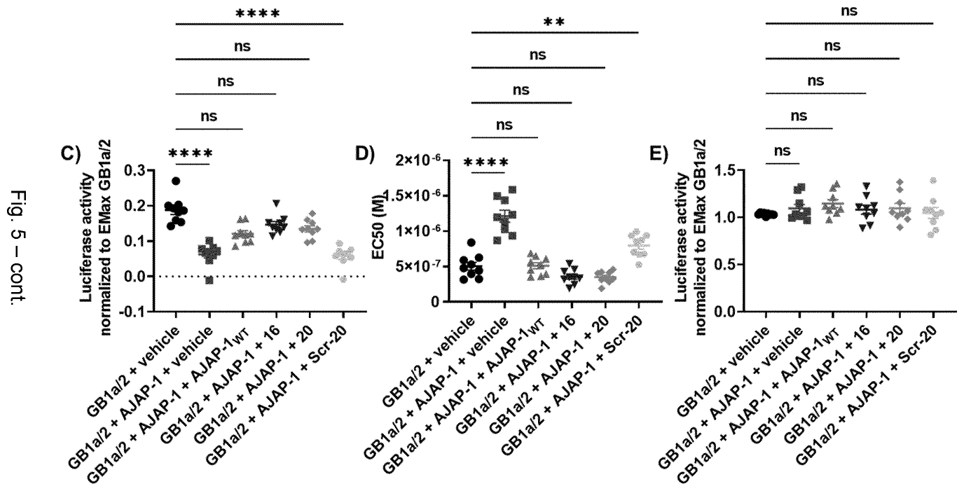

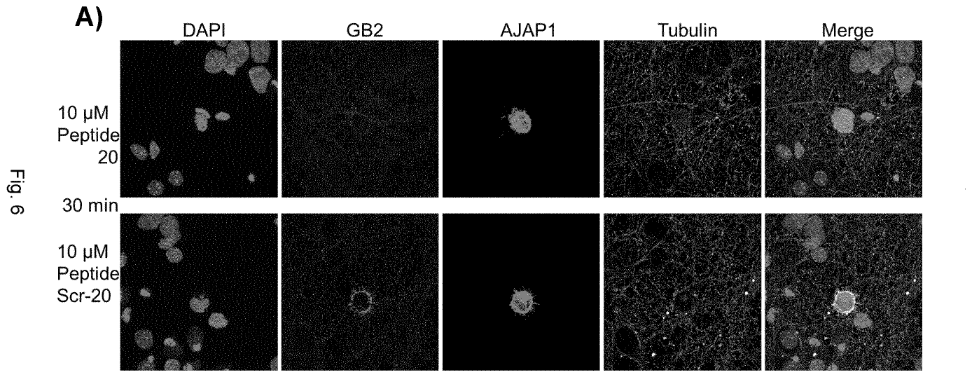

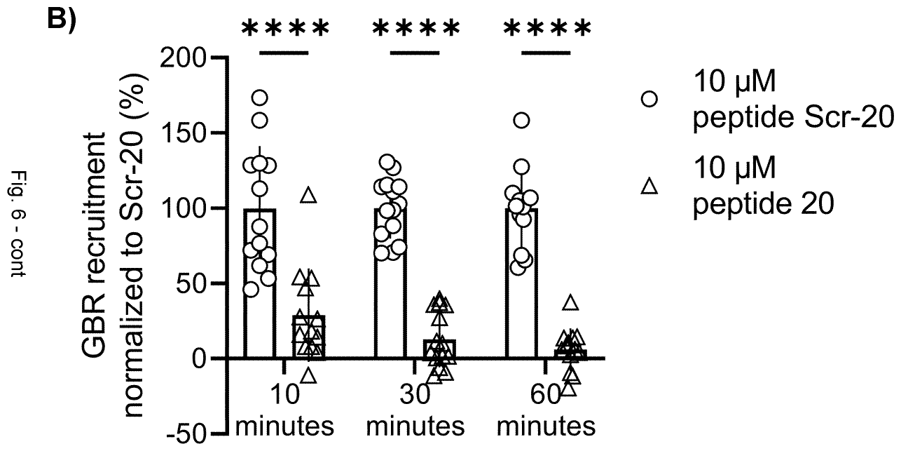

To elucidate whether our developed peptides AJAP-1_{WT}, 16 and 20 could interact and disrupt the trans-interaction between AJAP-1 and GB1a/2Rs, we used an established accumulation assay based on coupling GB1a/2Rs to phospholipase C (PLC) via chimeric Gα_{qi} (Conklin et al. 1993). PLC activity is then monitored with a serum responsive element-luciferase (SRE-Luciferase) reporter amplifying the GB1a/2Rs response (Yoo et al. 2017) (FIG. 5).

HEK293T cells stably expressing Gα_{q/i} were transiently transfected with Flag-GB1a, Flag-GB2 and SRE-FLuc with or without AJAP-1. In order to ensure GB1a/2 binding of AJAP-1 in trans, a pool of HEK293T-Gα_{q/i} cells expressing AJAP-1 was mixed with HEK293-Gα_{q/i} cells expressing Flag-GB1a, Flag-GB2 and SRE-FLuc. Transfected cells were distributed into 96-well microplates (Greiner Bio-One) at a density of 80,000 cells/well. After 24 h, the culture medium was replaced with Opti-MEM™-GlutaMAX™ Peptides were incubated in Opti-MEM™-GlutaMAX™ for 10 minutes. In presence of peptide, GB1a/2 receptors were activated with various concentrations of GABA for 6 h. FLuc activity in lysed cells was measured using the Luciferase® Assay Kit (Promega) and a Spark® microplate reader. Luminescence signals were adjusted by subtracting the luminescence obtained when expressing SRE-FLuc fusion proteins alone.

### Results

The presence of AJAP-1 protein in trans significantly reduced the constitutive activity of transcellular GB1a/2Rs and shifted the EC₅₀ towards a higher GABA concentration without affecting the maximum efficacy (Eₘₐₓ) (FIG. 5). The addition of 10 μM AJAP-1_{WT}, 16 or 20 in presence of AJAP-1 elevated basal activity of GB1a/2Rs (FIG. 5) and decreased EC₅₀ values (FIG. 5) of GABA activation at trans-cellular GB1a/2Rs to similar levels as receptors in absence of AJAP-1. Noteworthy, the developed low nanomolar affinity peptides (16 and 20) reduced the negative allosteric modulation effects of AJAP-1 at GB1a/2Rs in trans to a greater extent than AJAP-1_{WT} peptide, highlighting the importance of peptide optimization (FIG. 5). In comparison, when treated with a 10 μM solution of the scrambled control peptide Scr-20 in the presence of AJAP-1, trans-cellular GB1a/2Rs still showed significant decreased basal activity (FIG. 5) and increased EC₅₀ values of GABA at GB1a/2Rs (FIG. 5). Overall, all peptides (AJAP-1WT, 16, 20 and Scr-20) did not affect the Eₘₐₓ values of GABA-activated GB1a/2Rs in the absence of AJAP-1 (FIG. 5).

Peptides AJAP-1_{WT}, 16 and 20 specifically disrupt the transcellular AJAP-1/GB1a/2Rs interaction and reduce the negative allosteric modulation effects of AJAP-1 at GB1a/2Rs, and thus modulate the pharmacological profiles of GABA at GB1a/2Rs.

### Example 11. Synthesis of Cyclic Peptides

Linear peptides comprising two cysteine residues were solubilized in a solution consisting of 50% acetonitrile (ACN) and NH₄CO₃ buffer (pH=8.5) at a concentration of 1 mM. Subsequently, the linker, Xylylene dibromide, for cysteine cross-linking was dissolved in ACN at a concentration of 12 mM. The ACN solution of Xylylene dibromide was added dropwise into the peptide solution at room temperature under vigorous stirring until it reached a 1.2-fold excess relative to the quantity of peptides.

For linear peptides containing a single cysteine and chloroacetic acid-capped N-terminus, the cyclization reaction was initiated by dissolving these peptides in a solution comprising 50% ACN and NH₄CO₃ buffer (pH=8.5) at a concentration of 1 mM. Subsequently, the conversion of linear peptides into cyclic peptides was monitored using Ultra-Performance Liquid Chromatography (UPLC) and Liquid Chromatography-Mass Spectrometry (LC-MS). Upon completion of cyclization, the crude cyclized peptides were purified through Reversed-Phase High-Performance Liquid Chromatography (RP-HPLC).

Cyclic peptides generated are disclosed in table 17 of the present disclosure.

Sequence overview

SEQ ID NO: 4: EDDSDVWWG, is APP(203-211)
SEQ ID NO: 5: EFIAWGPTG, is AJAP-1(179-187)
SEQ ID NO: 6: AIVWGPTVS, is PIANP(99-107)
SEQ ID NO: 7: DVWWG, is APP(207-211)
SEQ ID NO: 8: FIAWG, is AJAP-1(180-184)
SEQ ID NO: 9: is AIVWG, is PIANP(99-103)
SEQ ID NO: 28: YGRKKRRQRRR is cell-penetrating peptide Tat
SEQ ID NO: 29: RRRRRRRR is cell-penetrating peptide polyR (R8)
SEQ ID NO: 30: RRRRRRR is cell-penetrating peptide polyR (R7)
SEQ ID NO: 31: RRRRRR is cell-penetrating peptide polyR (R6)
SEQ ID NO: 32: RRRRR is cell-penetrating peptide polyR (R5)
SEQ ID NO: 33: RRRR is cell-penetrating peptide polyR (R4)
SEQ ID NO: 60: Rigid Linker (RL) EAAAK
SEQ ID NO: 61: Flexible Linker (FL): GGGGS
SEQ ID NO: 62: EFIAWGP
SEQ ID NO: 63: IVWGPT
E X₁ X₂ X₃ X₄ X₅ X₆ W X₇ X₈ X₉ X₁₀ D X₁₁ D (SEQ ID NO: 64)

All peptides listed in any one of Table 3 to table 16 are N-terminally acetylated and C-terminally amidated.

TABLE 3
SPOT hit (epitopes mapping) validation and
previously reported peptides

| | Kᵢ (nM) | |
|---|---|---|
| Sequence | mean ± SEM | SEQ ID NO: |
| DDSDVWWGG | 2627 ± 97 | 65 |
| DDSDVWWGGADTDYADG | 687 ± 17 | 66 |
| EEDDSDVWWGGADTDYAD | 244 ± 13 | 67 |
| DSADAEEDDSDVWWG | 5030 ± 171 | 68 |
| SADAEEDDSDVWWGG | 1302 ± 28 | 69 |
| ADAEEDDSDVWWGGA | 1528 ± 46 | 70 |
| EDDSDVWWGGADTDY | 581 ± 21 | 71 |
| TTETEFIAWGPTGDE | 73 ± 1 | 72 |
| TETEFIAWGPTGDED | 23 ± 1 | 73 |
| ETEFIAWGPTGDEDA | 39 ± 1 | 74 |
| PWAIVWGPTVSREDG | 465 ± 16 | 75 |
| WAIVWGPTVSREDGG | 1347 ± 49 | 76 |
| AIVWGPTVSREDGGD | 637 ± 45 | 77 |

TABLE 4
Truncation and elongation scans of APP epitope

| | Kᵢ (nM) | SEQ |
|---|---|---|
| Sequence | mean ± SEM | ID NO: |
| DSADAEEDDSDVWWGGADTDYAD | 513 ± 17 | 78 |
| SADAEEDDSDVWWGGADTDYAD | 284 ± 5 | 79 |
| ADAEEDDSDVWWGGADTDYAD | 500 ± 16 | 80 |
| DAEEDDSDVWWGGADTDYAD | 247 ± 7 | 81 |
| AEEDDSDVWWGGADTDYAD | 237 ± 8 | 82 |
| EEDDSDVWWGGADTDYAD | 244 ± 13 | 83 |
| EEDDSDVWWGGADTDYADG | 343 ± 17 | 84 |
| EEDDSDVWWGGADTDYADGG | 341 ± 6 | 85 |
| EEDDSDVWWGGADTDYADGGE | 351 ± 26 | 86 |
| EEDDSDVWWGGADTDYADGGED | 356 ± 11 | 87 |
| EEDDSDVWWGGADTDYADGGEDK | 392 ± 14 | 88 |
| GGADTDYAD | >62500 | 89 |
| WGGADTDYAD | >62500 | 90 |
| WWGGADTDYAD | >62500 | 91 |
| VWWGGADTDYAD | 60837 ± 5215 | 92 |
| DVWWGGADTDYAD | 6105 ± 307 | 93 |
| SDVWWGGADTDYAD | 2950 ± 82 | 94 |
| DSDVWWGGADTDYAD | 1497 ± 66 | 95 |
| DDSDVWWGGADTDYAD | 517 ± 16 | 96 |
| EDDSDVWWGGADTDYAD | 281 ± 4 | 97 |
| EEDDSDVWWGGADTDYAD | 233 ± 7 | 98 |
| EEDDSDVWWGGADTDYA | 299 ± 8 | 99 |
| EEDDSDVWWGGADTDY | 301 ± 7 | 100 |
| EEDDSDVWWGGADTD | 341 ± 10 | 101 |
| EEDDSDVWWGGADT | 585 ± 10 | 102 |
| EEDDSDVWWGGAD | 534 ± 13 | 103 |
| EEDDSDVWWGGA | 1021 ± 18 | 104 |
| EEDDSDVWWGG | 1754 ± 7 | 105 |
| EEDDSDVWWG | 12802 ± 415 | 106 |
| EEDDSDVWW | 1786 ± 16 | 107 |
| EEDDSDVW | >62500 | 108 |

TABLE 5
Elongation and Truncation scan of AJAP-1 binding
epitope

| | Kᵢ (nM) | |
|---|---|---|
| Sequence | mean ± SEM | SEQ ID NO: |
| RPTTETEFIAWGPTGDED | 58 ± 0.4 | 109 |
| PTTETEFIAWGPTGDED | 30 ± 0.5 | 110 |
| TTETEFIAWGPTGDED | 32 ± 1.4 | 111 |
| TETEFIAWGPTGDED | 26 ± 0.3 | 112 |
| TETEFIAWGPTGDEDA | 36 ± 1.7 | 113 |
| TETEFIAWGPTGDEDAL | 47 ± 2.0 | 114 |
| AWGPTGDED | >125000 | 115 |
| IAWGPTGDED | 38000 ± 430 | 116 |
| FIAWGPTGDED | 118 ± 1 | 117 |
| EFIAWGPTGDED | 112 ± 1 | 118 |
| TEFIAWGPTGDED | 102 ± 4 | 119 |
| ETEFIAWGPTGDED | 32 ± 1 | 120 |
| TETEFIAWGPTGDED | 26 ± 1 | 121 |
| TETEFIAWGPTGDE | 69 ± 1 | 122 |
| TETEFIAWGPTGD | 97 ± 7 | 123 |
| TETEFIAWGPTG | 248 ± 7 | 124 |
| TETEFIAWGPT | 236 ± 9 | 125 |
| TETEFIAWGP | 574 ± 7 | 126 |
| TETEFIAWG | 2470 ± 170 | 127 |
| TETEFIAW | 1010 ± 67 | 128 |

TABLE 6
Elongation and Truncation scan of PIANP binding
epitope

| | Kᵢ (nM) | SEQ |
|---|---|---|
| Sequence | mean ± SEM | ID NO: |
| PSSQYPWAIVWGPTVSREDG | >62500 | 129 |
| SSQYPWAIVWGPTVSREDG | 29900 ± 2000 | 130 |
| SQYPWAIVWGPTVSREDG | 762 ± 13 | 131 |
| QYPWAIVWGPTVSREDG | 650 ± 9 | 132 |
| YPWAIVWGPTVSREDG | 433 ± 2 | 133 |
| PWAIVWGPTVSREDG | 463 ± 10 | 134 |
| PWAIVWGPTVSREDGG | 561 ± 21 | 135 |
| PWAIVWGPTVSREDGGD | 716 ± 6 | 136 |
| PWAIVWGPTVSREDGGDP | 597 ± 7 | 137 |
| PWAIVWGPTVSREDGGDPN | 579 ± 21 | 138 |
| PWAIVWGPTVSREDGGDPNS | 706 ± 16 | 139 |
| VWGPTVSREDG | 1740 ± 62 | 140 |
| IVWGPTVSREDG | 15700 ± 210 | 141 |
| AIVWGPTVSREDG | 5550 ± 11 | 142 |
| WAIVWGPTVSREDG | >62500 | 143 |
| PWAIVWGPTVSREDG | 29900 ± 2000 | 144 |
| PWAIVWGPTVSRED | 762 ± 13 | 145 |
| PWAIVWGPTVSRE | 650 ± 9 | 146 |
| PWAIVWGPTVSR | 433 ± 2 | 147 |
| PWAIVWGPTVS | 463 ± 10 | 148 |
| PWAIVWGPTV | 561 ± 21 | 149 |
| PWAIVWGPT | 716 ± 6 | 150 |
| PWAIVWGP | 597 ± 7 | 151 |
| PWAIVWG | 579 ± 21 | 152 |
| PWAIVW | 706 ± 16 | 153 |

TABLE 7
Ala-, D-amino acids and N-methylation
scans of APPWT

| | Kᵢ (nM) | SEQ ID |
|---|---|---|
| Sequence | mean ± SEM | NO: |
| ADDSDVWWGGADTD | 803 ± 5 | 154 |
| EADSDVWWGGADTD | 1238 ± 65 | 155 |
| EDASDVWWGGADTD | 1832 ± 54 | 156 |
| EDDADVWWGGADTD | 660 ± 21 | 157 |
| EDDSAVWWGGADTD | 3076 ± 22 | 158 |
| EDDSDAWWGGADTD | >62.5 μM | 159 |
| EDDSDVAWGGADTD | 8786 ± 92 | 160 |
| EDDSDVWAGGADTD | >62.5 μM | 161 |
| EDDSDVWWAGADTD | 485 ± 19 | 162 |
| EDDSDVWWGAADTD | 731 ± 17 | 163 |
| EDDSDVWWGGAATD | 653 ± 25 | 164 |
| EDDSDVWWGGADAD | 480 ± 8 | 165 |
| EDDSDVWWGGADTA | 786 ± 29 | 166 |
| eDDSDVWWGGADTD | 483 ± 4 | 167 |
| EdDSDVWWGGADTD | 567 ± 21 | 168 |
| EDdSDVWWGGADTD | 736 ± 15 | 169 |
| EDDsDVWWGGADTD | 752 ± 30 | 170 |
| EDDSdVWWGGADTD | 1632 ± 21 | 171 |
| EDDSDvWWGGADTD | >62.5 μM | 172 |
| EDDSDVwWGGADTD | >62.5 μM | 173 |
| EDDSDVWwGGADTD | >62.5 μM | 174 |
| EDDSDVWWaGADTD | 8703 ± 532 | 175 |
| EDDSDVWWGaADTD | 1213 ± 18 | 176 |
| EDDSDVWWGGaDTD | 755 ± 9 | 177 |
| EDDSDVWWGGAdTD | 637 ± 10 | 178 |
| EDDSDVWWGGADtD | 469 ± 11 | 179 |
| EDDSDVWWGGADTd | 529 ± 8 | 180 |
| (N-Me-E)DDSDVWWGGADTD | 670 ± 6 | 181 |
| E(N-Me-D)DSDVWWGGADTD | 655 ± 24 | 182 |
| ED(N-Me-D)SDVWWGGADTD | 671 ± 27 | 183 |
| EDD(N-Me-S)DVWWGGADTD | 1671 ± 32 | 184 |
| EDDS(N-Me-D)VWWGGADTD | 3759 ± 64 | 185 |
| EDDSD(N-Me-V)WWGGADTD | 12255 ± 375 | 186 |
| EDDSDV(N-Me-W)WGGADTD | 85559 ± 10133 | 187 |
| EDDSDVW(N-Me-W)GGADTD | 1382 ± 16 | 188 |
| EDDSDVWW(N-Me-G)GADTD | 18855 ± 554 | 189 |
| EDDSDVWWG(N-Me-G)ADTD | 161 ± 2 | 190 |
| EDDSDVWWGG(N-Me-A)DTD | 704 ± 11 | 191 |
| EDDSDVWWGGA(N-Me-D)TD | 403 ± 14 | 192 |
| EDDSDVWWGGAD(N-Me-T)D | 503 ± 13 | 193 |
| EDDSDVWWGGADT(N-Me-D) | 565 ± 23 | 194 |

TABLE 8
Ala-, D-amino acids and N-methylation
scans of AJAP-1_{WT}

| | Kᵢ (nM) | SEQ ID |
|---|---|---|
| Sequence | mean ± SEM | NO: |
| ATEFIAWGPTGDED | 53 ± 2 | 195 |
| EAEFIAWGPTGDED | 34 ± 3 | 196 |
| ETAFIAWGPTGDED | 49 ± 2 | 197 |
| ETEAIAWGPTGDED | 688 ± 11 | 198 |
| ETEFAAWGPTGDED | >6.25 μM | 199 |
| ETEFIAAGPTGDED | >6.25 μM | 200 |
| ETEFIAWAPTGDED | 196 ± 6 | 201 |
| ETEFIAWGATGDED | 291 ± 6 | 202 |
| ETEFIAWGPAGDED | 89 ± 2 | 203 |
| ETEFIAWGPTADED | 20 ± 1 | 204 |
| ETEFIAWGPTGAED | 50 ± 1 | 205 |
| ETEFIAWGPTGDAD | 38 ± 1 | 206 |
| ETEFIAWGPTGDEA | 44 ± 1 | 207 |
| eTEFIAWGPTGDED | 46 ± 2 | 208 |
| EtEFIAWGPTGDED | 56 ± 1 | 209 |
| ETeFIAWGPTGDED | 178 ± 8 | 210 |
| ETEfIAWGPTGDED | 930 ± 47 | 211 |
| ETEFIAWGPTGDED | >6.25 μM | 212 |
| ETEFlaWGPTGDED | >6.25 μM | 213 |
| ETEFIAwGPTGDED | >6.25 μM | 214 |
| ETEFIAWaPTGDED | 5402 ± 1117 | 215 |
| ETEFIAWGpTGDED | 352 ± 4 | 216 |
| ETEFIAWGPIGDED | 48 ± 2 | 217 |
| ETEFIAWGPTaDED | 54 ± 4 | 218 |
| ETEFIAWGPTGdED | 29 ± 1 | 219 |
| ETEFIAWGPTGDeD | 44 ± 1 | 220 |
| ETEFIAWGPTGDEd | 39 ± 1 | 221 |
| (N-Me-E)TEFIAWGPTGDED | 35 ± 1 | 222 |
| E(N-Me-T)EFIAWGPTGDED | 37 ± 2 | 223 |
| ET(N-Me-E)FIAWGPTGDED | 159 ± 3 | 224 |
| ETE(N-Me-F)IAWGPTGDED | 252 ± 5 | 225 |
| ETEF(N-Me-I)AWGPTGDED | 33 ± 1 | 226 |
| ETEFI(N-Me-A)WGPTGDED | >6.25 μM | 227 |
| ETEFIA(N-Me-W)GPTGDED | 46 ± 2 | 228 |
| ETEFIAW(N-Me-G)PTGDED | >6.25 μM | 229 |
| ETEFIAWG(N-Me-G)TGDED | 32 ± 2 | 230 |
| ETEFIAWGP(N-Me-T)GDED | 77 ± 3 | 231 |
| ETEFIAWGPT(N-Me-G)DED | 132 ± 5 | 232 |
| ETEFIAWGPTG(N-Me-D)ED | 45 ± 2 | 233 |
| ETEFIAWGPTGD(N-Me-E)D | 31 ± 1 | 234 |
| ETEFIAWGPTGDE(N-Me-D) | 29 ± 1 | 235 |

TABLE 9
Ala-, D-amino acids and N-methylation
scans of PIANPWT

| | Kᵢ (nM) | SEQ ID |
|---|---|---|
| Sequence | mean ± SEM | NO: |
| AWAIVWGPTVSRED | 552 ± 20 | 236 |
| PAAIVWGPTVSRED | 911 ± 13 | 237 |
| PWAAVWGPTVSRED | 46641 ± 4835 | 238 |
| PWAIAWGPTVSRED | 1462 ± 14 | 239 |
| PWAIVAGPTVSRED | >62.5 μM | 240 |
| PWAIVWAPTVSRED | 877 ± 19 | 241 |
| PWAIVWGATVSRED | 1505 ± 34 | 242 |
| PWAIVWGPAVSRED | 1029 ± 26 | 243 |
| PWAIVWGPTASRED | 318 ± 3 | 244 |
| PWAIVWGPTVARED | 480 ± 4 | 245 |
| PWAIVWGPTVSAED | 341 ± 7 | 246 |
| PWAIVWGPTVSRAD | 600 ± 22 | 247 |
| PWAIVWGPTVSREA | 572 ± 9 | 248 |
| pWAIVWGPTVSRED | 555 ± 27 | 249 |
| PwAIVWGPTVSRED | 1232 ± 73 | 250 |
| PWaIVWGPTVSRED | 14102 ± 124 | 251 |
| PWAIVWGPTVSRED | >62.5 μM | 252 |
| PWAIvWGPTVSRED | >62.5 μM | 253 |
| PWAIVwGPTVSRED | >62.5 μM | 254 |
| PWAIVWaPTVSRED | 18118 ± 62 | 255 |
| PWAIVWGpTVSRED | 6858 ± 185 | 256 |
| PWAIVWGPtVSRED | 1300 ± 10 | 257 |
| PWAIVWGPTvSRED | 686 ± 19 | 258 |
| PWAIVWGPTVsRED | 552 ± 4 | 259 |
| PWAIVWGPTVSrED | 605 ± 15 | 260 |
| PWAIVWGPTVSReD | 760 ± 25 | 261 |
| PWAIVWGPTVSREd | 725 ± 3 | 262 |
| P(N-Me-W)AIVWGPTVSRED | 833 ± 12 | 263 |
| PW(N-Me-A)IVWGPTVSRED | 7233 ± 130 | 264 |
| PWA(N-Me-I)VWGPTVSRED | 370 ± 5 | 265 |
| PWAI(N-Me-V)WGPTVSRED | 6999 ± 100 | 266 |
| PWAIV(N-Me-W)GPTVSRED | 636 ± 6 | 267 |
| PWAIVW(N-Me-G)PTVSRED | 58309 ± 4414 | 268 |
| PWAIVWG(N-Me-G)TVSRED | 525 ± 22 | 269 |
| PWAIVWGP(N-Me-T)VSRED | 1265 ± 27 | 270 |
| PWAIVWGPT(N-Me-V)SRED | 1778 ± 87 | 271 |
| PWAIVWGPTV(N-Me-S)RED | 1045 ± 1 | 272 |
| PWAIVWGPTVS(N-Me-R)ED | 502 ± 15 | 273 |
| PWAIVWGPTVSR(N-Me-E)D | 523 ± 13 | 274 |
| PWAIVWGPTVSRE(N-Me-D) | 434 ± 10 | 275 |

TABLE 10
Deep mutational scanning validation
variants of APP_{WT}

| | Kᵢ (nM) | SEQ ID |
|---|---|---|
| Sequence | mean ± SEM | NO: |
| KDDSDVWWGGADTD | 1525 ± 53 | 276 |
| EDDWDVWWGGADTD | 257 ± 4 | 277 |
| EDDSFVWWGGADTD | 142 ± 6 | 278 |
| EDDSIVWWGGADTD | 243 ± 3 | 279 |
| EDDSYVWWGGADTD | 191 ± 10 | 280 |
| EDDSWVWWGGADTD | 100 ± 3 | 281 |
| EDDSDIWWGGADTD | 401 ± 7 | 282 |
| EDDSDVDWGGADTD | 4804 ± 43 | 283 |
| EDDSDVIWGGADTD | 951 ± 20 | 284 |
| EDDSDVYWGGADTD | 3601 ± 49 | 285 |
| EDDSDVWWDGADTD | 429 ± 7 | 286 |
| EDDSDVWWHGADTD | 270 ± 4 | 287 |
| EDDSDVWWYGADTD | 108 ± 1 | 288 |
| EDDSDVWWGPADTD | 332 ± 13 | 289 |
| EDDSDVWWGGHDTD | 346 ± 6 | 290 |
| EDDSDVWWGGSDTD | 204 ± 4 | 291 |
| EDDSDVWWGGYDTD | 255 ± 11 | 292 |
| EDDSDVWWGGADED | 337 ± 10 | 293 |
| EDDSDVWWGGADHD | 739 ± 9 | 294 |
| EDDSDVWWGGADWD | 459 ± 21 | 295 |

TABLE 11
Deep mutational
scanning validation
variants of AJAP-1_{WT}

| | Kᵢ (nM) | SEQ ID |
|---|---|---|
| | mean ± SEM | NO: |
| Sequence | scanning | variants |
| RTEFIAWGPTGDED | 87 ± 3 | 296 |
| NTEFIAWGPTGDED | 62 ± 3 | 297 |
| VTEFIAWGPTGDED | 67 ± 1 | 298 |
| EIEFIAWGPTGDED | 22 ± 1 | 299 |
| EVEFIAWGPTGDED | 28 ± 1 | 300 |
| ETDFIAWGPTGDED | 26 ± 1 | 301 |
| ETHFIAWGPTGDED | 58 ± 4 | 302 |
| ETSFIAWGPTGDED | 34 ± 1 | 303 |
| ETEWIAWGPTGDED | 35 ± 2 | 304 |
| ETEYIAWGPTGDED | 18 ± 1 | 305 |
| ETEKIAWGPTGDED | >6.25 μM | 306 |
| ETEFIPWGPTGDED | >6.25 μM | 307 |
| ETEFIDWGPTGDED | 9 ± 1 | 308 |
| ETEFIHWGPTGDED | 24 ± 1 | 309 |
| ETEFIVWGPTGDED | 30 ± 1 | 310 |
| ETEFIAWHPTGDED | 240 ± 7 | 311 |
| ETEFIAWYPTGDED | 199 ± 10 | 312 |
| ETEFIAWGPFGDED | 40 ± 1 | 313 |
| ETEFIAWGPHGDED | 38 ± 1 | 314 |
| ETEFIAWGPSGDED | 32 ± 1 | 315 |
| ETEFIAWGPTFDED | 46 ± 2 | 316 |
| ETEFIAWGPTHDED | 30 ± 1 | 317 |
| ETEFIAWGPTRDED | 32 ± 1 | 318 |
| ETEFIAWGPTGHED | 60 ± 4 | 319 |
| ETEFIAWGPTGYED | 41 ± 1 | 320 |
| ETEFIAWGPTGDDD | 27 ± 2 | 321 |
| ETEFIAWGPTGDFD | 41 ± 2 | 322 |
| ETEFIAWGPTGDHD | 39 ± 2 | 323 |
| ETEFIAWGPTGDND | 39 ± 1 | 324 |
| ETEFIAWGPTGDPD | 34 ± 2 | 325 |
| ETEFIAWGPTGDRD | 54 ± 2 | 326 |
| ETEFIAWGPTGDVD | 39 ± 1 | 327 |
| ETEFIAWGPTGDWD | 54 ± 1 | 328 |
| ETEFIAWGPTGDYD | 40 ± 1 | 329 |
| ETEFIAWGPTGDEF | 48 ± 1 | 330 |
| ETEFIAWGPTGDEH | 39 ± 2 | 331 |
| ETEFIAWGPTGDEN | 43 ± 2 | 332 |
| ETEFIAWGPTGDEP | 41 ± 2 | 333 |
| ETEFIAWGPTGDEQ | 37 ± 2 | 334 |
| ETEFIAWGPTGDEV | 40 ± 2 | 335 |
| ETEFIAWGPTGDEW | 54 ± 2 | 336 |
| ETEFIAWGPTGDEY | 39 ± 1 | 337 |

TABLE 12
Deep mutational scanning validation
variants of PIANP_{WT}

| | | SEQ | Pep- |
|---|---|---|---|
| | Kᵢ (nM) | ID | tide |
| Sequence | mean ± SEM | NO: | no.: |
| VWAIVWGPTVSRED | 412 ± 20 | 338 | |
| DWAIVWGPTVSRED | 338 ± 1 | 339 | |
| PDAIVWGPTVSRED | 673 ± 7 | 340 | |
| DAIVWGPTVSRED | 715 ± 15 | 341 | |
| DAIVWGPTVSRED | 208 ± 10 | 342 | |
| PHAIVWGPTVSRED | 1503 ± 50 | 343 | |
| PSAIVWGPTVSRED | 906 ± 20 | 344 | |
| PFAIVWGPTVSRED | 792 ± 11 | 345 | |
| PWFIVWGPTVSRED | 461 ± 10 | 346 | |
| PWIIVWGPTVSRED | 339 ± 8 | 347 | |
| PWVIVWGPTVSRED | 302 ± 4 | 348 | |
| PWYIVWGPTVSRED | 336 ± 2 | 349 | |
| PWFIVWGPTVSDED | 192 ± 4 | 350 | |
| PWAIDWGPTVSRED | 1302 ± 16 | 351 | |
| PWAIVWGPTSSRED | 383 ± 3 | 352 | |
| PWAIVWGPTDSRED | 417 ± 6 | 353 | |
| PWAIVWGPTVDRED | 379 ± 9 | 354 | |
| PWAIVWGPTVSDED | 327 ± 6 | 355 | |
| AIVWGPTVSRED | 683 ± 41 | 356 | |
| AIVWGPTVSRED* | 773 ± 11 | 357 | |
| FIVWGPTVSRED | 367 ± 18 | 358 | |
| FIVWGPTVSRED* | 503 ± 9 | 359 | |

TABLE 13
Peptides for affinity maturation

| | | SEQ |
|---|---|---|
| | Kᵢ (nM) | ID |
| Sequence | mean ± SEM | NO: |
| ETEFIAWGPTVSRED | 148 ± 6 | 360 |
| PWAIVWGPTGDED | 222 ± 1 | 361 |
| PWAIVWG(N-Me-G)TVSRED | 525 ± 22 | 362 |
| EDDSWVWWG(N-Me-G)ADTD | 36 ± 1 | 363 |
| EDDSDVWWGPTGDED | 62 ± 1 | 364 |
| ETEFIAWGGADTD | 345 ± 9 | 365 |
| ETEFIAWYGADTD | 95 ± 5 | 366 |
| ETEFIAWYPADTD | 190 ± 3 | 367 |
| ETEFIAWYGPADTD | 182 ± 2 | 368 |
| ETEFIAWYPTGDED | 199 ± 10 | 369 |
| ETEFIAWYGPTGDED | 195 ± 7 | 370 |
| ETEFIAWG(N-Me-G)TGDED | 32 ± 2 | 371 |
| EDDSDVWWG(N-Me-G)ADTD | 161 ± 2 | 372 |
| EDDSDVWWY(N-Me-G)ADTD | 201 ± 6 | 373 |
| EDDSDVWWYG(N-Me-G)ADTD | 164 ± 2 | 374 |
| EDDSDVWWY(N-Me-G)TGDED | 142 ± 3 | 375 |
| EDDSDVWWYG(N-Me-G)TGDED | 138 ± 5 | 376 |
| EDDSWIWWYGADTD | 18 ± 1 | 377 |
| EDDSWIWWGPTGDED | 16 ± 1 | 378 |
| EDDSWIDWYGADTD | 11 ± 0.1 | 379 |
| EDDSWIDWGPTGDED | 5.6 ± 0.7 | 380 |
| EDDDWIDWYDSDDD | 3.2 ± 0.2 | 381 |
| EDDDWIDWGPTSDDD | 4.0 ± 0.3 | 382 |
| EDDYIDWYDSDDD | 4.6 ± 0.3 | 383 |
| EDDYIDWGPTSDDD | 3.8 ± 0.2 | 384 |

TABLE 14
CPPs tagged peptides

| | Ki (nM) | SEQ ID |
|---|---|---|
| Sequence | mean ± SEM | NO: |
| RRRRRRRREDDDWIDWYDSDDD | 107.3 ± 1 | 385 |
| EDDDWIDWYDSDDDRRRRRRRR | 65.3 ± 1 | 386 |
| YGRKKRRQRRREDDDWIDWYDSDDD | 83.4 ± 1 | 387 |
| EDDDWIDWYDSDDDYGRKKRRQRRR | 70.5 ± 0.3 | 388 |
| RRRRRRRREDDDWIDWGPTSDDD | 58.1 ± 3 | 389 |
| EDDDWIDWGPTSDDDRRRRRRRR | 123.4 ± 4 | 390 |
| YGRKKRRQRRREDDDWIDWGPTSDDD | 49.9 ± 2 | 391 |
| EDDDWIDWGPTSDDDYGRKKRRQRRR | 78.5 ± 1 | 392 |
| RRRRRRRREAAAKEDDDWIDWYDSDDD | 314 ± 4 | 393 |
| RRRRRRRRGGGGSEDDDWIDWYDSDDD | 280 ± 2 | 394 |
| RRRRRRRREAAAKEDDDWIDWGPTSDDD | 105 ± 2 | 395 |
| RRRRRRRRGGGGSEDDDWIDWGPTSDDD | 140 ± 4 | 396 |
| EDDDWIDWGPTSDDDEAAAKRRRRRRRR | 139 ± 2 | 397 |
| EDDDWIDWGPTSDDDGGGGSRRRRRRRR | 147 ± 9 | 398 |
| EDDDWIDWYDSDDDEAAAKRRRRRRRR | 99.5 ± 4 | 399 |
| EDDDWIDWYDSDDDGGGGSRRRRRRRR | 117.9 ± 3 | 400 |
| RRRRRRRREDDDWIDWYDSDDD | 107.3 ± 2 | 401 |
| RRRRRRREDDDWIDWYDSDDD | 89.1 ± 3 | 402 |
| RRRRRREDDDWIDWYDSDDD | 70.9 ± 1 | 403 |
| RRRRREDDDWIDWYDSDDD | 39.5 ± 2 | 404 |
| RRRREDDDWIDWYDSDDD | 20.6 ± 0.2 | 405 |
| RRREDDDWIDWYDSDDD | 6.4 ± 0.1 | 406 |
| RRRRRRRREDDDWIDWGPTSDDD | 58.1 ± 3 | 407 |
| RRRRRRREDDDWIDWGPTSDDD | 65 ± 2 | 408 |
| RRRRRREDDDWIDWGPTSDDD | 43.4 ± 1 | 409 |
| RRRRREDDDWIDWGPTSDDD | 32 ± 1 | 410 |
| RRRREDDDWIDWGPTSDDD | 15 ± 1 | 411 |
| RRREDDDWIDWGPTSDDD | 9 ± 0.2 | 412 |
| RRRREDDD(2Nala)IDWYDSDDD | 15 ± 1 | 413 |
| RRRREDDD(2Nala)IDWGPTSDDD | 13 ± 1 | 414 |
| RRRR(2Nala)EDSDDYDDIDDDW | 142537 ± 2504 | 415 |

TABLE 15
CA-tagged peptides for CAPA

| | Kᵢ (nM) | SEQ ID |
|---|---|---|
| Sequence | mean ± SEM | NO: |
| CA-ETEFIAWGPTGDED | 45 ± 4 | 416 |
| CA-EDDDWIDWYDSDDD | 153 ± 21 | 417 |
| CA-EDDDWIDWGPTSDDD | 21 ± 2 | 418 |
| CA-EDDD(2Nala)IDWYDSDDD | 146 ± 7 | 419 |
| CA-EDDD(2Nala)IDWGPTSDDD | 72 ± 6 | 420 |
| CA-(2Nala)EDSDDYDDIDDDW | 118 ± 15 | 421 |
| CA-RRRR(2Nala)EDSDDYDDIDDDW | 0.6 ± 0.2 | 422 |
| CA-RRRREDDD(2Nala)IDWYDSDDD | 1.1 ± 0.2 | 423 |
| CA-RRRREDDD(2Nala)IDWGPTSDDD | 1.2 ± 0.3 | 424 |
| CA-RRRRRRRREDDDWIDWGPTSDDD | 1.3 ± 0.7 | 425 |
| CA-RRRRRRRREDDDWIDWYDSDDD | 1.5 ± 0.9 | 426 |
| CA-EDDDWIDWGPTSDDDRRRRRRRR | 17 ± 1.6 | 427 |
| CA-EDDDWIDWYDSDDDRRRRRRRR | 17 ± 0.2 | 428 |
| CA-YGRKKRRQRRREDDDWIDWGPTSDDD | 2.4 ± 1.0 | 429 |
| CA-YGRKKRRQRRREDDDWIDWYDSDDD | 0.8 ± 0.1 | 430 |
| CA-EDDDWIDWYDSDDDYGRKKRRQRRR | 19 ± 0.4 | 431 |
| CA-EDDDWIDWGPTSDDDYGRKKRRQRRR | 6.1 ± 0.8 | 432 |
| CA-EDDDWIDWYDSDDDEAAAKRRRRRRRR | 10 ± 1.5 | 433 |
| CA-EDDDWIDWYDSDDDGGGGSRRRRRRRR | 9.0 ± 1.2 | 434 |
| CA-RRRRRRRREDDDWIDWYDSDDD | 1.5 ± 0.9 | 435 |
| CA-RRRRRRREDDDWIDWYDSDDD | 0.6 ± 0.4 | 436 |
| CA-RRRRRREDDDWIDWYDSDDD | 0.5 ± 0.3 | 437 |
| CA-RRRRREDDDWIDWYDSDDD | 0.5 ± 0.2 | 438 |
| CA-RRRREDDDWIDWYDSDDD | 1.0 ± 0.4 | 439 |
| CA-RRREDDDWIDWYDSDDD | 5.3 ± 2.2 | 440 |
| CA-RRRRRRRREDDDWIDWGPTSDDD | 1.3 ± 0.7 | 441 |
| CA-RRRRRRREDDDWIDWGPTSDDD | 0.9 ± 0.3 | 442 |
| CA-RRRRRREDDDWIDWGPTSDDD | 0.6 ± 0.1 | 443 |
| CA-RRRRREDDDWIDWGPTSDDD | 0.6 ± 0.1 | 444 |
| CA-RRRREDDDWIDWGPTSDDD | 1.4 ± 0.3 | 445 |
| CA-RRREDDDWIDWGPTSDDD | 8.3 ± 2.0 | 446 |

TABLE 16
ncAAs incorporated peptides

| | Kᵢ (nM) | SEQ ID |
|---|---|---|
| Sequence | mean ± SEM | NO: |
| EDDS(1Nala)VWWGGADTD | 69 ± 3 | 447 |
| EDDS(2Nala)VWWGGADTD | 43 ± 1 | 448 |
| EDDS(Obtyr)VWWGGADTD | 28 ± 1 | 449 |
| EDDS(Oetyr)VWWGGADTD | 46 ± 2 | 450 |
| EDDS(Omtyr)VWWGGADTD | 101 ± 4 | 451 |
| EDDS(Fphe)VWWGGADTD | 117 ± 2 | 452 |
| EDDS(Aphe)VWWGGADTD | 184 ± 3 | 453 |
| EDDS(Buphe)VWWGGADTD | 269 ± 3 | 454 |
| EDDS(3Pal)VWWGGADTD | 373 ± 7 | 455 |
| EDDS(4Pal)VWWGGADTD | 424 ± 13 | 456 |
| EDDSDVWW(1Nala)GADTD | 114 ± 1 | 457 |
| EDDSDVWW(2Nala)GADTD | 75 ± 1 | 458 |
| EDDSDVWW(Obtyr)GADTD | 88 ± 4 | 459 |
| EDDSDVWW(Oetyr)GADTD | 113 ± 2 | 460 |
| EDDSDVWW(Omtyr)GADTD | 109 ± 4 | 461 |
| EDDSDVWW(Aphe)GADTD | 102 ± 1 | 462 |
| EDDSDVW(1Nala)GGADTD | 499 ± 17 | 463 |
| EDDSDVW(2Nala)GGADTD | 814 ± 10 | 464 |
| EDDSDVW(Obtyr)GGADTD | 3863 ± 62 | 465 |
| ETEFI(Cya)WGPTGDED | 12 ± 0.3 | 466 |
| (2Nala)EDSDDYDDIDDDW | 31222 ± 989 | 467 |
| EDDD(2Nala)IDWYDSDDD | 1.5 ± 0.2 | 468 |
| EDDD(2Nala)IDWGPTSDDD | 5.3 ± 0.2 | 469 |
| EDDD(Obtyr)IDWYDSDDD | 9.3 ± 0.2 | 470 |
| EDDD(Obtyr)IDWGPTSDDD | 9.5 ± 0.3 | 471 |
| EDDD(Oetyr)IDWYDSDDD | 5.3 ± 0.2 | 472 |
| EDDD(Oetyr)IDWGPTSDDD | 6.5 ± 0.4 | 473 |

SEQ ID NO: 1:
Ac-EDDSDVWWGGADTD-NH₂, is (APP_{WT})
SEQ ID NO: 2:
Ac-ETEFIAWGPTGDED-NH₂, is (AJAP-1_{WT})
SEQ ID NO: 3:
Ac-PWAIVWGPTVSRED-NH₂, is (PIANP_{WT})
SEQ ID NO: 10:
TAMRA-TETEFIAWGPTGDED is AJAP-1_{probe}
SEQ ID NO: 11:
DSADAEEDDSDVWWG
SEQ ID NO: 12:
SADAEEDDSDVWWGG
SEQ ID NO: 13:
EDDSDVWWGGADTDY
SEQ ID NO: 15:
Ac-TETEFIAWGPTGDED-NH₂
SEQ ID NO: 16:
Ac-EDDDWIDWYDSDDD-NH₂
SEQ ID NO: 17:
Ac-EDDDWIDWGPTSDDD-NH₂
SEQ ID NO: 18:
Ac-EDDYIDWYDSDDD-NH₂
SEQ ID NO: 19:
Ac-EDDYIDWGPTSDDD-NH₂
SEQ ID NO: 20:
Ac-EDDD(2Nala)IDWYDSDDD-NH₂
SEQ ID NO: 21:
Ac-EDDD(2Nala)IDWGPTSDDD-NH₂
SEQ ID NO: 22:
Ac-EDDYTDWYDSDDD-NH₂
SEQ ID NO: 23:
Ac-EDDYTDWGPTSDDD-NH₂
SEQ ID NO: 24:
Ac-ETEFIAWGPTADED-NH₂
SEQ ID NO: 25:
Ac-ETEYIAWGPTGDED-NH₂
SEQ ID NO: 26:
Ac-ETEFIDWGPTGDED-NH₂
SEQ ID NO: 27:
Ac-EDDSWIWWGPTGDED-NH₂
SEQ ID NO: 28:
Ac-EDDSWIWWYGADTD-NH₂
SEQ ID NO: 29:
Ac-EDDSWIDWGPTGDED-NH₂
SEQ ID NO: 30:
Ac-EDDSWIDWYGADTD-NH₂
SEQ ID NO: 31:
Ac-ETEFI(Cya)WGPTGDED-NH₂
SEQ ID NO: 32:
Ac-EDDD(Oetyr)IDWYDSDDD-NH₂
SEQ ID NO: 33:
Ac-EDDD(Obtyr)IDWYDSDDD-NH₂
SEQ ID NO: 34:
Ac-EDDD(Oetyr)IDWGPTSDDD-NH₂
SEQ ID NO: 35:
Ac-EDDD(Obtyr)IDWGPTSDDD-NH₂
SEQ ID NO: 36:
Ac-RRRRRRRREDDDWIDWYDSDDD-NH₂
SEQ ID NO: 37:
Ac-RRRREDDDWIDWYDSDDD-NH₂
SEQ ID NO: 38:
Ac-RRREDDDWIDWYDSDDD-NH₂
SEQ ID NO: 39:
Ac-RRRRRRRREDDDWIDWGPTSDDD-NH₂
SEQ ID NO: 40:
Ac-RRRREDDDWIDWGPTSDDD-NH₂
SEQ ID NO: 41:
Ac-RRREDDDWIDWGPTSDDD-NH₂
SEQ ID NO: 42:
Ac-RRRREDDD(2Nala)IDWYDSDDD-NH₂
SEQ ID NO: 43:
Ac-RRRREDDD(2Nala)IDWGPTSDDD-NH₂
SEQ ID NO: 22:
Ac-EDDYTDWYDSDDD-NH₂
SEQ ID NO: 23:
Ac-EDDYTDWGPTSDDD-NH₂
SEQ ID NO: 27:
Ac-EDDSWIWWGPTGDED-NH₂
SEQ ID NO: 28:
Ac-EDDSWIWWYGADTD-NH₂
SEQ ID NO: 48:
Ac-EDDSDVWWGPTGDED-NH₂ (Peptide X)
SEQ ID NO: 49:
Ac-ETEFIAWGGADTD-NH₂ (Peptide Y)
SEQ ID NO: 50:
Ac-ETEFIAWYGADTD-NH₂ (Peptide 37)
SEQ ID NO: 51:
Ac-ETEFIAWG(N-Me-G)TGDED-NH₂ (Peptide 38)
Tat-16 Ac-YGRKKRRQRRREDDDWIDWYDSDDD-NH₂
16-Tat Ac-DDDWIDWYDSDDDYGRKKRRQRRR-NH₂
SEQ ID NO: 55:
Ac-EEDDSDVWWGGADTDYAD-NH₂ (Peptide 36)

TABLE 17
Overview of synthesized cyclic peptides. Data are displayed
as calculated Kᵢ values (mean + SEM, n ≥ 3).

| | | Affinity, | SEQ |
|---|---|---|---|
| No. | Sequence (structure) | (Kᵢ) | ID no |
| ZC509 | 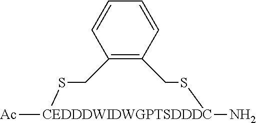 | 140 ± 2.3 | 475 |
| ZC510 | 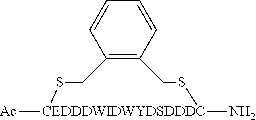 | 100 ± 1.7 | 476 |
| ZC513 | 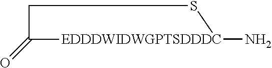 | 19 ± 0.5 | 477 |
| ZC514 | 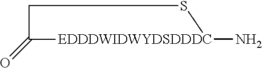 | 4.3 ± 0.3 | 478 |

### REFERENCES

Heaney, C. F.; Kinney, J. W., Role of GABA (B) receptors in learning and memory and neurological disorders. Neurosci Biobehav Rev 2016, 63, 1-28.
Bowery, N. G.; Bettler, B.; Froestl, W.; Gallagher, J. P.; Marshall, F.; Raiteri, M.; Bonner, T. I.; Enna, S. J., International Union of Pharmacology. XXXIII. Mammalian γ-aminobutyric acid_{B} receptors: structure and function. Pharmacol. Rev. 2002, 54 (2), 247-264.
Wedin, G. P.; Hornfeldt, C. S.; Ylitalo, L. M., The clinical development of γ-hydroxybutyrate (GHB). Curr. Drug Saf. 2006, 1 (1), 99-106.
Bay, T.; Eghorn, L. F.; Klein, A. B.; Wellendorph, P., GHB receptor targets in the CNS: Focus on high-affinity binding sites. Biochem Pharmacol 2014, 87 (2), 220-228.
Gassmann, M.; Bettler, B., Regulation of neuronal GABA_{B} receptor functions by subunit composition. Nat. Rev. Neurosci. 2012, 13 (6), 380-394.
Fritzius, T.; Bettler, B., The organizing principle of GABA_{B} receptor complexes: Physiological and pharmacological implications. Basic Clin. Pharmacol. Toxicol. 2019.
Bettler, B.; Tiao, J. Y., Molecular diversity, trafficking and subcellular localization of GABA_{B} receptors. Pharmacol. Ther. 2006, 110 (3), 533-543.
Bettler, B.; Kaupmann, K.; Mosbacher, J.; Gassmann, M., Molecular structure and physiological functions of GABA_{B} receptors. Physiol. Rev. 2004, 84, 835-867.
Vigot, R.; Barbieri, S.; Brauner-Osborne, H.; Turecek, R.; Shigemoto, R.; Zhang, Y. P.; Lujan, R.; Jacobson, L. H.; Biermann, B.; Fritschy, J. M.; Vacher, C. M.; Muller, M.; Sansig, G.; Guetg, N.; Cryan, J. F.; Kaupmann, K.; Gassmann, M.; Oertner, T. G.; Bettler, B., Differential compartmentalization and distinct functions of GABA(B) receptor variants. Neuron 2006, 50 (4), 589-601.
Blein, S.; Ginham, R.; Uhrin, D.; Smith, B. O.; Soares, D. C.; Veltel, S.; McIlhinney, R. A. J.; White, J. H.; Barlow, P. N., Structural analysis of the complement control protein (CCP) modules of GABA_{B} receptor 1a J. Biol. Chem. 2004, 279 (46), 48292-48306.
Ulrich, D.; Bettler, B., GABA_{B} receptors: synaptic functions and mechanisms of diversity. Curr. Opin. Neurobiol. 2007, 17 (3), 298-303.
Perez-Garci, E.; Gassmann, M.; Bettler, B.; Larkum, M. E., The GABA_{B1b} isoform mediates long-lasting inhibition of dendritic Ca²⁺ spikes in layer 5 somatosensory pyramidal neurons. Neuron 2006, 50 (4), 603-616.
Schwenk, J.; Perez-Garci, E.; Schneider, A.; Kollewe, A.; Gauthier-Kemper, A.; Fritzius, T.; Raveh, A.; Dinamarca, M. C.; Hanuschkin, A.; Bildl, W.; Klingauf, J.; Gassmann, M.; Schulte, U.; Bettler, B.; Fakler, B., Modular composition and dynamics of native GABA_{B} receptors identified by high-resolution proteomics. Nat. Neurosci. 2016, 19 (2), 233-242.
Dinamarca, M. C.; Raveh, A.; Schneider, A.; Fritzius, T.; Fruh, S.; Rem, P. D.; Stawarski, M.; Lalanne, T.; Turecek, R.; Choo, M.; Besseyrias, V.; Bildl, W.; Bentrop, D.; Staufenbiel, M.; Gassmann, M.; Fakler, B.; Schwenk, J.; Bettler, B., Complex formation of APP with GABA_{B} receptors links axonal trafficking to amyloidogenic processing. Nat. Commun. 2019, 10, 1331.
Rice, H. C.; De Malmazet, D.; Schreurs, A.; Frere, S.; Van Molle, I.; Volkov, A. N.; Creemers, E.; Vertkin, I.; Nys, J.; Ranaivoson, F. M.; Comoletti, D.; Savas, J. N.; Remaut, H.; Balschun, D.; Wierda, K. D.; Slutsky, I.; Farrow, K.; De Strooper, B.; De Wit, J., Secreted amyloid-β precursor protein functions as a GABA_{B}R1a ligand to modulate synaptic transmission. Science 2019, 363 (6423), eaao4827.
Fritzius, T.; Bettler, B., The organizing principle of GABA_{B} receptor complexes: Physiological and pharmacological implications. Basic Clin. Pharmacol. Toxicol. 2019.
Conklin, B. R.; Farfel, Z.; Lustig, K. D.; Julius, D.; Bourne, H. R., Substitution of three amino acids switches receptor specificity of Gq alpha to that of Gi alpha. Nature 1993, 363 (6426), 274-6.
Yoo, Y.; Jung, J.; Lee, Y. N.; Lee, Y.; Cho, H.; Na, E.; Hong, J.; Kim, E.; Lee, J. S.; Lee, J. S.; Hong, C.; Park, S. Y.; Wie, J.; Miller, K.; Shur, N.; Clow, C.; Ebel, R. S.; DeBrosse, S. D.; Henderson, L. B.; Willaert, R.; Castaldi, C.; Tikhonova, I.; Bilguvar, K.; Mane, S.; Kim, K. J.; Hwang, Y. S.; Lee, S. G.; So, I.; Lim, B. C.; Choi, H. J.; Seong, J. Y.; Shin, Y. B.; Jung, H.; Chae, J. H.; Choi, M., GABBR2 mutations determine phenotype in rett syndrome and epileptic encephalopathy. Annals of Neurology 2017, 82 (3), 466-478.
Garbutt J C, Approval of Baclofen for Alcohol Use Disorders in France: A Perspective From the United States. Alcohol and Alcoholism. 2020, 55 (1) 48.

1. A peptide comprising or consisting of the sequence 1 2 3 4 5 6 7 8 9 10 11 12 13 14 15
(SEQ ID NO: 64)
E X₁ X₂ X₃ X₄ X₅ X₆ W X₇ X₈ X₉ X₁₀ D X₁₁ D
wherein
X₁ is Aspartic acid (D) or Threonine (T),
X₂ is Aspartic acid (D) or Glutamic acid (E),
X₃ is S, Aspartic acid (D), Tyrosine (Y) or Phenylalanine (F),
X₄ is absent, Tryptophan (W), 3-(2-naphthyl)-L-alanine (2Nala), O-ethyl-L-tyrosine (Oetyr), O-benzyl-L-tyrosine (Obtyr), or L-Cysteic acid (Cya),
X₅ is Isoleucine (I) or Threonine (T),
X₆ is Aspartic acid (D), Tryptophan (W) or Alanine (A),
X₇ is Tyrosine (Y) or Glycine (G),
X₈ is Glycine (G), Proline (P) or Aspartic acid (D),
X₉ is Alanine (A), Threonine (T) or Serine(S),
X₁₀ is absent, Glycine (G), Serine(S) or Alanine (A),
X₁₁ is Aspartic acid (D), Threonine (T) or Glutamic acid (E),
or a functional variant thereof, wherein the functional variant comprises 1 or 2 individual amino acid substitutions

2. The peptide according to claim 1, wherein the functional variant comprises 1 or 2 individual amino acid substitutions, such as 2 individual amino acid substitutions, such as 1 individual amino acid substitution.

3. The peptide according to anyone of the preceding claims, wherein said peptide is an inhibitor of GB1a/2Rs (heterodimeric gamma-amino butyric acid (GABA)_{B1a}/GABA_{B2} receptor).

4. The peptide according to anyone of the preceding claims, wherein said peptide is an inhibitor of protein-protein interactions at GB1a/2Rs.

5. The peptide according to anyone of the preceding claims, wherein X₁ is D.

6. The peptide according to anyone of the preceding claims, wherein X₂ is D.

7. The peptide according to anyone of the preceding claims, wherein X₃ is S, D or Y, such as wherein X₃ is D or Y, such as wherein X₃ is D.

8. The peptide according to anyone of the preceding claims, wherein X₄ is absent, W, 2-Nal, O-Ey-Y or Obtyr.

9. The peptide according to anyone of the preceding claims, wherein X₄ is absent.

10. The peptide according to anyone of the preceding claims, wherein X₄ is W, 2-Nal, O-Ey-Y or Obtyr, such as wherein X₄ is W or 2-Nal.

11. The peptide according to anyone of the preceding claims, wherein X₅ is I or T.

12. The peptide according to anyone of the preceding claims, wherein X₆ is D.

13. The peptide according to anyone of the preceding claims, wherein X₈ is P or D.

14. The peptide according to anyone of the preceding claims, wherein X₉ is A or S, and wherein X₁₀ is absent.

15. The peptide according to anyone of the preceding claims, wherein X₉ is T and X₁₀ is G, S.

16. The peptide according to anyone of the preceding claims, wherein X₁₀ is G, S or absent, such as wherein X₁₀ is G or S, such as wherein X₁₀ is absent.

17. The peptide according to anyone of the preceding claims, wherein X₁₀ is absent and wherein X₁₁ is D or T, such as wherein X₁₀ is absent and wherein X₁₁ is D.

18. The peptide according to anyone of the preceding claims, wherein X₁₁ is D or E and wherein X₁₀ is G, S.

19. The peptide according to anyone of the preceding claims, wherein the peptide or functional variant thereof comprises at least 8 amino acid residues, such as 9 amino acid residues, such as 10 amino acid residues, such as 11 amino acid residues, such as 12 amino acid residues, such as 13 amino acid residues, such as 14 amino acid residues, such as 15 amino acid residues, such as 16 amino acid residues, such as 17 amino acid residues, such as 18 amino acid residues, such as 19 amino acid residues, such as 20 amino acid residues, such as 21 amino acid residues, such as 22 amino acid residues, such as 23 amino acid residues, such as 24 amino acid residues, such as 25 amino acid residues, such as 26 amino acid residues, such as 27 amino acid residues.

20. The peptide according to anyone of the preceding claims, wherein the peptide or functional variant thereof comprises between 8 and 35 amino acid residues, such as between 8 and 30 amino acid residues, such as between 8 and 27 amino acid residues, such as between 8 and 23 amino acid residues.

21. The peptide according to anyone of the preceding claims, wherein the peptide is selected from the group consisting of:
SEQ ID NO: 15: TETEFIAWGPTGDED
SEQ ID NO: 16: EDDDWIDWYDSDDD
SEQ ID NO: 17: EDDDWIDWGPTSDDD
SEQ ID NO: 18: EDDYIDWYDSDDD
SEQ ID NO: 19: EDDYIDWGPTSDDD
SEQ ID NO: 20: EDDD(2Nala)IDWYDSDDD
SEQ ID NO: 21: EDDD(2Nala)IDWGPTSDDD
SEQ ID NO: 22: EDDYTDWYDSDDD
SEQ ID NO: 23: EDDYTDWGPTSDDD
SEQ ID NO: 24: ETEFIAWGPTADED
SEQ ID NO: 25: ETEYIAWGPTGDED
SEQ ID NO: 26: ETEFIDWGPTGDED
SEQ ID NO: 27: EDDSWIWWGPTGDED
SEQ ID NO: 28: EDDSWIWWYGADTD
SEQ ID NO: 29: EDDSWIDWGPTGDED
SEQ ID NO: 30: EDDSWIDWYGADTD
SEQ ID NO: 31: ETEFI(Cya)WGPTGDED
SEQ ID NO: 32: EDDD(Oetyr)IDWYDSDDD
SEQ ID NO: 33: EDDD(Obtyr)IDWYDSDDD
SEQ ID NO: 34: EDDD(Oetyr)IDWGPTSDDD
SEQ ID NO: 35: EDDD(Obtyr)IDWGPTSDDD
or a functional variant thereof, wherein the functional variant comprises 1 or 2 individual amino acid substitutions.

22. The peptide according to anyone of the preceding claims, wherein the peptide is N-terminally acetylated (Ac—N-terminus).

23. The peptide according to anyone of the preceding claims, wherein the peptide is C-terminally amidated (—NH₂) or wherein the C-terminus is a carboxylic acid.

24. The peptide according to anyone of the preceding claims, wherein the peptide is conjugated to a cell-penetrating peptides (CPP).

25. The peptide according to anyone of the preceding claims, wherein the CPP is conjugated to the N-terminus or to the C-terminus of the peptide.

26. The peptide according to anyone of the preceding claims, wherein the CPP is conjugated to the peptide via a linker, such as via a peptidic linker.

27. The peptide according to anyone of the preceding claims, wherein the CPP is
Tat,
(SEQ ID NO: 55)
YGRKKRRQRRR,
R8,
(SEQ ID NO: 56)
RRRRRRRR,
R7,
(SEQ ID NO: 57)
RRRRRRR,
R6,
(SEQ ID NO: 58)
RRRRRR,
R5,
(SEQ ID NO: 59)
RRRRR,
R4,
(SEQ ID NO: 60)
RRRR,
R3, RRR,
or
R2, RR.

28. The peptide according to anyone of the preceding claims, wherein the CPP is
R8,
(SEQ ID NO: 56)
RRRRRRRR
or
R4,
(SEQ ID NO: 60)
RRRR.

29. The peptide according to anyone of the preceding claims, wherein the peptide is selected from the group consisting of:
SEQ ID NO: 15: Ac-TETEFIAWGPTGDED-NH₂
SEQ ID NO: 16: Ac-EDDDWIDWYDSDDD-NH₂
SEQ ID NO: 17: Ac-EDDDWIDWGPTSDDD-NH₂
SEQ ID NO: 18: Ac-EDDYIDWYDSDDD-NH₂
SEQ ID NO: 19: Ac-EDDYIDWGPTSDDD-NH₂
SEQ ID NO: 20: Ac-EDDD(2Nala)IDWYDSDDD-NH₂
SEQ ID NO: 21: Ac-EDDD(2Nala)IDWGPTSDDD-NH₂
SEQ ID NO: 22: Ac-EDDYTDWYDSDDD-NH₂
SEQ ID NO: 23: Ac-EDDYTDWGPTSDDD-NH₂
SEQ ID NO: 24: Ac-ETEFIAWGPTADED-NH₂
SEQ ID NO: 25: Ac-ETEYIAWGPTGDED-NH₂
SEQ ID NO: 26: Ac-ETEFIDWGPTGDED-NH₂
SEQ ID NO: 27: Ac-EDDSWIWWGPTGDED-NH₂
SEQ ID NO: 28: Ac-EDDSWIWWYGADTD-NH₂
SEQ ID NO: 29: Ac-EDDSWIDWGPTGDED-NH₂
SEQ ID NO: 30: Ac-EDDSWIDWYGADTD-NH₂
SEQ ID NO: 31: Ac-ETEFI(Cya)WGPTGDED-NH₂
SEQ ID NO: 32: Ac-EDDD(Oetyr)IDWYDSDDD-NH₂
SEQ ID NO: 33: Ac-EDDD(Obtyr)IDWYDSDDD-NH₂
SEQ ID NO: 34: Ac-EDDD(Oetyr)IDWGPTSDDD-NH₂
SEQ ID NO: 35: Ac-EDDD(Obtyr)IDWGPTSDDD-NH₂
SEQ ID NO: 36: Ac-RRRRRRRREDDDWIDWYDSDDD-NH₂
SEQ ID NO: 37: Ac-RRRREDDDWIDWYDSDDD-NH₂
SEQ ID NO: 38: Ac-RRREDDDWIDWYDSDDD-NH₂
SEQ ID NO: 39: Ac-RRRRRRRREDDDWIDWGPTSDDD-NH₂
SEQ ID NO: 40: Ac-RRRREDDDWIDWGPTSDDD-NH₂
SEQ ID NO: 41: Ac-RRREDDDWIDWGPTSDDD-NH₂
SEQ ID NO: 42: Ac-RRRREDDD(2Nala)IDWYDSDDD-NH₂
SEQ ID NO: 43: Ac-RRRREDDD(2Nala)IDWGPTSDDD-NH₂
or a functional variant thereof, wherein the functional variant comprises 1 or 2 individual amino acid substitutions.

30. The peptide according to anyone of the preceding claims, wherein the peptide is a cyclic polypeptide.

31. The peptide according to anyone of the preceding claims, wherein the peptide is “head-to-tail” cyclized.

32. The peptide according to anyone of the preceding claims, wherein the peptide is “side chain-to-side chain” cyclized.

33. The peptide according to anyone of the preceding claims, wherein the peptide is “tail-to-side chain” cyclized.

34. The peptide according to anyone of the preceding claims, wherein the peptide is “side chain-to-head” cyclized.

35. The peptide according to anyone of the preceding claims, wherein the peptide is back bone cyclized.

36. The peptide according to anyone of the preceding claims, wherein the N-terminal amino acid residue is linked to the C-terminal amino acid residue via a peptide bond.

37. The peptide according to anyone of the preceding claims, wherein the N-terminal amino acid residue is linked to the C-terminal amino acid residue via a non-peptide chemical linker.

38. The peptide according to anyone of the preceding claims, wherein the peptides further comprises:
a. two cysteines linked via a non-peptide chemical linker; and/or
b. a cysteine linked to the N-terminal amino acid residue of the peptide.

39. The peptide according to anyone of the preceding claims, wherein the peptide further comprises two cysteines.

40. The peptide according to anyone of the preceding claims, wherein the peptide further comprise a cysteine at the N-terminal.

41. The peptide according to anyone of the preceding claims, wherein the peptide further comprises a cysteine at the C-terminal.

42. The peptide according to anyone of the preceding claims, wherein the peptide further comprises a cysteine at the N-terminal and a cysteine at the C-terminal.

43. The peptide according to anyone of the preceding claims, wherein a linker connects two cysteines within the peptide of the present disclosure.

44. The peptide according to anyone of the preceding claims, wherein a linker connects a cysteine at the N-terminal amino acid residue and a cysteine at the C-terminal amino acid residue of the peptide.

45. The peptide according to anyone of the preceding claims, wherein the non-peptide chemical linker is:

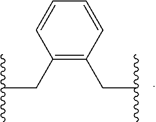 [SMILES: *Cc1ccccc1C*]

46. The peptide according to anyone of the preceding claims, wherein the peptide is selected from the group consisting of:

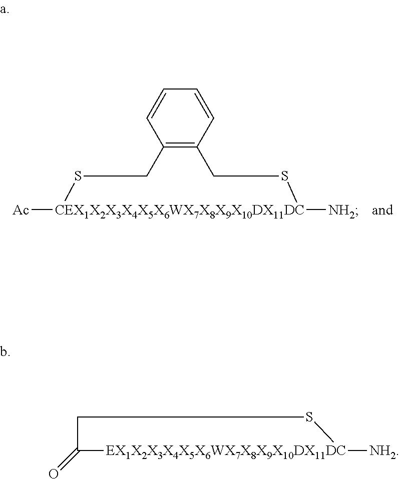

47. The peptide according to anyone of the preceding claims, wherein the peptide is

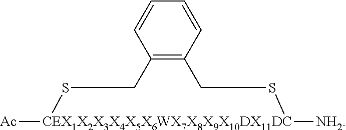

48. The peptide according to anyone of the preceding claims, wherein the peptide is

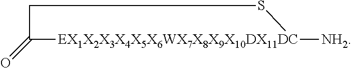

49. The peptide according to anyone of the preceding claims, wherein the peptide is

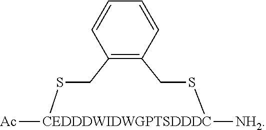

50. The peptide according to anyone of the preceding claims, wherein the peptide is

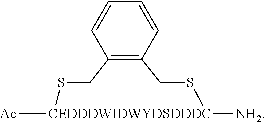

51. The peptide according to anyone of the preceding claims, wherein the peptide is

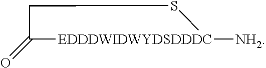

52. The peptide according to anyone of the preceding claims, wherein the peptide is

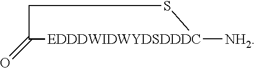

53. The peptide according to anyone of the preceding claims, wherein the peptide is conjugated to an additional moiety.

54. The peptide according to anyone of the preceding claims, wherein the peptide has binding affinity (Kᵢ) to sushi domain (SD1/2) protein of 20 nM or less.

55. The peptide according to anyone of the preceding claims, wherein the peptide has membrane permeability (CP₅₀) of 30 μM or less, such as of 10 μM or less.

56. The peptide according to anyone of the preceding claims, wherein the peptide has plasma stability (T_{1/2}) of 12 hours or more, such as of 16 hours or more, such as of 20 hours or more, such as of 24 hours or more, such as of 30 hours or more, such as of 36 hours or more, such as of 40 hours or more, such as of 48 hours or more.

57. The peptide according to anyone of the preceding claims, wherein the peptide is capable of inhibiting an interaction between a protein and a sushi domain (SD) of GB1a at presynaptic neurons.

58. The peptide according to anyone of the preceding claims, wherein the peptide is capable of inhibiting an interaction between:
sushi domain (SD) and adherence-junction associated protein 1 (AJAP1),
SD and PILRα-associated neural protein (PIANP), and/or
SD and amyloid precursor protein (APP).

59. A peptide according to anyone of the preceding claims for use as a medicament.

60. A peptide according to anyone of the preceding claims for use in the treatment of neurological and/or psychiatric disorders.

61. A peptide for use according to anyone of the preceding claims, wherein the neurological disorder is epilepsy, spasticity, schizophrenia, dystonia, neuropathic pain, narcolepsy, spasticity, cognitive deficits such as Mild cognitive impairment (MCI), autism spectrum disorder and/or substance-use disorders.

62. A peptide for use according to anyone of the preceding claims, wherein the psychiatric disorder is depression, anxiety and/or Attention deficit hyperactivity disorder (ADHD).

63. A method for treatment of neurological and/or psychiatric disorders comprising administration of a peptide according to anyone of the preceding claims to an individual in need thereof.

64. Use of a peptide according to anyone of the preceding claims for the manufacture of a medicament for the treatment of neurological and/or psychiatric disorders.

65. A medicament for use in the treatment or prevention of neurological and/or psychiatric disorders comprising a peptide according to anyone of the preceding claims.


SEQUENCE LISTING (480 sequences)

SEQ ID NO: 1 - protein, 14 aa, synthetic construct
  MOD_RES 1: note = ACETYLATION
  MOD_RES 14: note = AMIDATION
EDDSDVWWGGADTD

SEQ ID NO: 2 - protein, 14 aa, synthetic construct
  MOD_RES 1: note = ACETYLATION
  MOD_RES 14: note = AMIDATION
ETEFIAWGPTGDED

SEQ ID NO: 3 - protein, 14 aa, synthetic construct
  MOD_RES 1: note = ACETYLATION
  MOD_RES 14: note = AMIDATION
PWAIVWGPTVSRED

SEQ ID NO: 4 - protein, 9 aa, synthetic construct
EDDSDVWWG

SEQ ID NO: 5 - protein, 9 aa, synthetic construct
EFIAWGPTG

SEQ ID NO: 6 - protein, 9 aa, synthetic construct
AIVWGPTVS

SEQ ID NO: 7 - protein, 5 aa, synthetic construct
DVWWG

SEQ ID NO: 8 - protein, 5 aa, synthetic construct
FIAWG

SEQ ID NO: 9 - protein, 5 aa, synthetic construct
AIVWG

SEQ ID NO: 10 - protein, 15 aa, synthetic construct
  SITE 1: note = tetramethylrhodamine (TAMRA) attached to residue 1
TETEFIAWGPTGDED

SEQ ID NO: 11 - protein, 15 aa, synthetic construct
  MOD_RES 1: note = ACETYLATION
  MOD_RES 15: note = AMIDATION
DSADAEEDDSDVWWG

SEQ ID NO: 12 - protein, 15 aa, synthetic construct
  MOD_RES 1: note = ACETYLATION
  MOD_RES 15: note = AMIDATION
SADAEEDDSDVWWGG

SEQ ID NO: 13 - protein, 15 aa, synthetic construct
  MOD_RES 1: note = ACETYLATION
  MOD_RES 15: note = AMIDATION
EDDSDVWWGGADTDY

SEQ ID NO: 14 - protein, 15 aa, synthetic construct
  MOD_RES 1: note = ACETYLATION
  MOD_RES 15: note = AMIDATION
PWAIVWGPTVSREDG

SEQ ID NO: 15 - protein, 15 aa, synthetic construct
  MOD_RES 1: note = ACETYLATION
  MOD_RES 15: note = AMIDATION
TETEFIAWGPTGDED

SEQ ID NO: 16 - protein, 14 aa, synthetic construct
  MOD_RES 1: note = ACETYLATION
  MOD_RES 14: note = AMIDATION
EDDDWIDWYDSDDD

SEQ ID NO: 17 - protein, 15 aa, synthetic construct
  MOD_RES 1: note = ACETYLATION
  MOD_RES 15: note = AMIDATION
EDDDWIDWGPTSDDD

SEQ ID NO: 18 - protein, 13 aa, synthetic construct
  MOD_RES 1: note = ACETYLATION
  MOD_RES 13: note = AMIDATION
EDDYIDWYDSDDD

SEQ ID NO: 19 - protein, 14 aa, synthetic construct
  MOD_RES 1: note = ACETYLATION
  MOD_RES 14: note = AMIDATION
EDDYIDWGPTSDDD

SEQ ID NO: 20 - protein, 14 aa, synthetic construct
  MOD_RES 1: note = ACETYLATION
  MOD_RES 14: note = AMIDATION
  SITE 5: note = 3-(2-NAPHTHYL)-L-ALANINE
EDDDAIDWYDSDDD

SEQ ID NO: 21 - protein, 15 aa, synthetic construct
  MOD_RES 1: note = ACETYLATION
  MOD_RES 15: note = AMIDATION
  SITE 5: note = 3-(2-NAPHTHYL)-L-ALANINE
EDDDAIDWGPTSDDD

SEQ ID NO: 22 - protein, 13 aa, synthetic construct
  MOD_RES 1: note = ACETYLATION
  MOD_RES 13: note = AMIDATION
EDDYTDWYDSDDD

SEQ ID NO: 23 - protein, 14 aa, synthetic construct
  MOD_RES 1: note = ACETYLATION
  MOD_RES 14: note = AMIDATION
EDDYTDWGPTSDDD

SEQ ID NO: 24 - protein, 14 aa, synthetic construct
  MOD_RES 1: note = ACETYLATION
  MOD_RES 14: note = AMIDATION
ETEFIAWGPTADED

SEQ ID NO: 25 - protein, 14 aa, synthetic construct
  MOD_RES 1: note = ACETYLATION
  MOD_RES 14: note = AMIDATION
ETEYIAWGPTGDED

SEQ ID NO: 26 - protein, 14 aa, synthetic construct
  MOD_RES 1: note = ACETYLATION
  MOD_RES 14: note = AMIDATION
ETEFIDWGPTGDED

SEQ ID NO: 27 - protein, 15 aa, synthetic construct
  MOD_RES 1: note = ACETYLATION
  MOD_RES 15: note = AMIDATION
EDDSWIWWGPTGDED

SEQ ID NO: 28 - protein, 14 aa, synthetic construct
  MOD_RES 1: note = ACETYLATION
  MOD_RES 14: note = AMIDATION
EDDSWIWWYGADTD

SEQ ID NO: 29 - protein, 15 aa, synthetic construct
  MOD_RES 1: note = ACETYLATION
  MOD_RES 15: note = AMIDATION
EDDSWIDWGPTGDED

SEQ ID NO: 30 - protein, 14 aa, synthetic construct
  MOD_RES 1: note = ACETYLATION
  MOD_RES 14: note = AMIDATION
EDDSWIDWYGADTD

SEQ ID NO: 31 - protein, 14 aa, synthetic construct
  MOD_RES 1: note = ACETYLATION
  MOD_RES 14: note = AMIDATION
  SITE 6: note = L-CYSTEIC ACID
ETEFICWGPTGDED

SEQ ID NO: 32 - protein, 14 aa, synthetic construct
  MOD_RES 1: note = ACETYLATION
  MOD_RES 14: note = AMIDATION
  SITE 5: note = O-ETHYL-L-TYROSINE
EDDDYIDWYDSDDD

SEQ ID NO: 33 - protein, 14 aa, synthetic construct
  MOD_RES 1: note = ACETYLATION
  MOD_RES 14: note = AMIDATION
  SITE 5: note = O-BENZYL-L-TYROSINE
EDDDYIDWYDSDDD

SEQ ID NO: 34 - protein, 15 aa, synthetic construct
  MOD_RES 1: note = ACETYLATION
  MOD_RES 15: note = AMIDATION
  SITE 5: note = O-ETHYL-L-TYROSINE
EDDDYIDWGPTSDDD

SEQ ID NO: 35 - protein, 15 aa, synthetic construct
  MOD_RES 1: note = ACETYLATION
  MOD_RES 15: note = AMIDATION
  SITE 5: note = O-BENZYL-L-TYROSINE
EDDDYIDWGPTSDDD

SEQ ID NO: 36 - protein, 22 aa, synthetic construct
  MOD_RES 1: note = ACETYLATION
  MOD_RES 22: note = AMIDATION
RRRRRRRREDDDWIDWYDSDDD

SEQ ID NO: 37 - protein, 18 aa, synthetic construct
  MOD_RES 1: note = ACETYLATION
  MOD_RES 18: note = AMIDATION
RRRREDDDWIDWYDSDDD

SEQ ID NO: 38 - protein, 17 aa, synthetic construct
  MOD_RES 1: note = ACETYLATION
  MOD_RES 17: note = AMIDATION
RRREDDDWIDWYDSDDD

SEQ ID NO: 39 - protein, 23 aa, synthetic construct
  MOD_RES 1: note = ACETYLATION
  MOD_RES 23: note = AMIDATION
RRRRRRRREDDDWIDWGPTSDDD

SEQ ID NO: 40 - protein, 19 aa, synthetic construct
  MOD_RES 1: note = ACETYLATION
  MOD_RES 19: note = AMIDATION
RRRREDDDWIDWGPTSDDD

SEQ ID NO: 41 - protein, 18 aa, synthetic construct
  MOD_RES 1: note = ACETYLATION
  MOD_RES 18: note = AMIDATION
RRREDDDWIDWGPTSDDD

SEQ ID NO: 42 - protein, 18 aa, synthetic construct
  MOD_RES 1: note = ACETYLATION
  MOD_RES 18: note = AMIDATION
  SITE 9: note = 3-(2-NAPHTHYL)-L-ALANINE
RRRREDDDAIDWYDSDDD

SEQ ID NO: 43 - protein, 19 aa, synthetic construct
  MOD_RES 1: note = ACETYLATION
  MOD_RES 19: note = AMIDATION
  SITE 9: note = 3-(2-NAPHTHYL)-L-ALANINE
RRRREDDDAIDWGPTSDDD

SEQ ID NO: 44 - protein, 13 aa, synthetic construct
  MOD_RES 1: note = ACETYLATION
  MOD_RES 13: note = AMIDATION
EDDYTDWYDSDDD

SEQ ID NO: 45 - protein, 14 aa, synthetic construct
  MOD_RES 1: note = ACETYLATION
  MOD_RES 14: note = AMIDATION
EDDYTDWGPTSDDD

SEQ ID NO: 46 - protein, 15 aa, synthetic construct
  MOD_RES 1: note = ACETYLATION
  MOD_RES 15: note = AMIDATION
EDDSWIWWGPTGDED

SEQ ID NO: 47 - protein, 14 aa, synthetic construct
  MOD_RES 1: note = ACETYLATION
  MOD_RES 14: note = AMIDATION
EDDSWIWWYGADTD

SEQ ID NO: 48 - protein, 15 aa, synthetic construct
  MOD_RES 1: note = ACETYLATION
  MOD_RES 15: note = AMIDATION
EDDSDVWWGPTGDED

SEQ ID NO: 49 - protein, 13 aa, synthetic construct
  MOD_RES 1: note = ACETYLATION
  MOD_RES 13: note = AMIDATION
ETEFIAWGGADTD

SEQ ID NO: 50 - protein, 13 aa, synthetic construct
  MOD_RES 1: note = ACETYLATION
  MOD_RES 13: note = AMIDATION
ETEFIAWYGADTD

SEQ ID NO: 51 - protein, 14 aa, synthetic construct
  MOD_RES 1: note = ACETYLATION
  MOD_RES 14: note = AMIDATION
  MOD_RES 9: note = MeGly
ETEFIAWGGTGDED

SEQ ID NO: 52 - protein, 25 aa, synthetic construct
  MOD_RES 1: note = ACETYLATION
  MOD_RES 25: note = AMIDATION
YGRKKRRQRRREDDDWIDWYDSDDD

SEQ ID NO: 53 - protein, 24 aa, synthetic construct
  MOD_RES 1: note = ACETYLATION
  MOD_RES 24: note = AMIDATION
DDDWIDWYDSDDDYGRKKRRQRRR

SEQ ID NO: 54 - protein, 18 aa, synthetic construct
  MOD_RES 1: note = ACETYLATION
  MOD_RES 18: note = AMIDATION
EEDDSDVWWGGADTDYAD

SEQ ID NO: 55 - protein, 11 aa, synthetic construct
YGRKKRRQRRR

SEQ ID NO: 56 - protein, 8 aa, synthetic construct
RRRRRRRR

SEQ ID NO: 57 - protein, 7 aa, synthetic construct
RRRRRRR

SEQ ID NO: 58 - protein, 6 aa, synthetic construct
RRRRRR

SEQ ID NO: 59 - protein, 4 aa, synthetic construct
RRRR

SEQ ID NO: 60 - protein, 5 aa, synthetic construct
EAAAK

SEQ ID NO: 61 - protein, 5 aa, synthetic construct
GGGGS

SEQ ID NO: 62 - protein, 7 aa, synthetic construct
EFIAWGP

SEQ ID NO: 63 - protein, 6 aa, synthetic construct
IVWGPT

SEQ ID NO: 64 - protein, 15 aa, synthetic construct
  VARIANT 2: note = ASPARTIC ACID OR THREONINE
  VARIANT 3: note = ASPARTIC ACID OR GLUTAMIC ACID
  VARIANT 4: note = SERINE, ASPARTIC ACID, TYROSINE OR PHENYLALANINE
  VARIANT 5: note = MAY BE DELETED OR TRYPTOPHAN, 3-(2-NAPHTHYL)-L-ALANINE, O-ETHYL-L-TYROSINE, O-BENZYL-L-TYROSINE OR L-CYSTEIC ACID
  VARIANT 6: note = ISOLEUCINE OR THREONINE
  VARIANT 7: note = ASPARTIC ACID, TRYPTOPHAN OR ALANINE
  VARIANT 9: note = TYROSINE OR GLYCINE
  VARIANT 10: note = GLYCINE, PROLINE OR ASPARTIC ACID
  VARIANT 11: note = ALANINE, THREONINE OR SERINE
  VARIANT 12: note = MAY BE DELETED OR GLYCINE, SERINE OR ALANINE
  VARIANT 14: note = ASPARTIC ACID, THREONINE OR GLUTAMIC ACID
EXXXXXXWXXXXDXD

SEQ ID NO: 65 - protein, 9 aa, synthetic construct
  MOD_RES 1: note = ACETYLATED
  MOD_RES 9: note = AMINATED
DDSDVWWGG

SEQ ID NO: 66 - protein, 17 aa, synthetic construct
  MOD_RES 1: note = ACETYLATION
  MOD_RES 17: note = AMIDATION
DDSDVWWGGADTDYADG

SEQ ID NO: 67 - protein, 18 aa, synthetic construct
  MOD_RES 1: note = ACETYLATION
  MOD_RES 18: note = AMIDATION
EEDDSDVWWGGADTDYAD

SEQ ID NO: 68 - protein, 15 aa, synthetic construct
  MOD_RES 1: note = ACETYLATION
  MOD_RES 15: note = AMIDATION
DSADAEEDDSDVWWG

SEQ ID NO: 69 - protein, 15 aa, synthetic construct
  MOD_RES 1: note = ACETYLATION
  MOD_RES 15: note = AMIDATION
SADAEEDDSDVWWGG

SEQ ID NO: 70 - protein, 15 aa, synthetic construct
  MOD_RES 1: note = ACETYLATION
  MOD_RES 15: note = AMIDATION
ADAEEDDSDVWWGGA

SEQ ID NO: 71 - protein, 15 aa, synthetic construct
  MOD_RES 1: note = ACETYLATION
  MOD_RES 15: note = AMIDATION
EDDSDVWWGGADTDY

SEQ ID NO: 72 - protein, 15 aa, synthetic construct
  MOD_RES 1: note = ACETYLATION
  MOD_RES 15: note = AMIDATION
TTETEFIAWGPTGDE

SEQ ID NO: 73 - protein, 15 aa, synthetic construct
  MOD_RES 1: note = ACETYLATION
  MOD_RES 15: note = AMIDATION
TETEFIAWGPTGDED

SEQ ID NO: 74 - protein, 15 aa, synthetic construct
  MOD_RES 1: note = ACETYLATION
  MOD_RES 15: note = AMIDATION
ETEFIAWGPTGDEDA

SEQ ID NO: 75 - protein, 15 aa, synthetic construct
  MOD_RES 1: note = ACETYLATION
  MOD_RES 15: note = AMIDATION
PWAIVWGPTVSREDG

SEQ ID NO: 76 - protein, 15 aa, synthetic construct
  MOD_RES 1: note = ACETYLATION
  MOD_RES 15: note = AMIDATION
WAIVWGPTVSREDGG

SEQ ID NO: 77 - protein, 15 aa, synthetic construct
  MOD_RES 1: note = ACETYLATION
  MOD_RES 15: note = AMIDATION
AIVWGPTVSREDGGD

SEQ ID NO: 78 - protein, 23 aa, synthetic construct
  MOD_RES 1: note = ACETYLATION
  MOD_RES 23: note = AMIDATION
DSADAEEDDSDVWWGGADTDYAD

SEQ ID NO: 79 - protein, 22 aa, synthetic construct
  MOD_RES 1: note = ACETYLATION
  MOD_RES 22: note = AMIDATION
SADAEEDDSDVWWGGADTDYAD

SEQ ID NO: 80 - protein, 21 aa, synthetic construct
  MOD_RES 1: note = ACETYLATION
  MOD_RES 21: note = AMIDATION
ADAEEDDSDVWWGGADTDYAD

SEQ ID NO: 81 - protein, 20 aa, synthetic construct
  MOD_RES 1: note = ACETYLATION
  MOD_RES 20: note = AMIDATION
DAEEDDSDVWWGGADTDYAD

SEQ ID NO: 82 - protein, 19 aa, synthetic construct
  MOD_RES 1: note = ACETYLATION
  MOD_RES 19: note = AMIDATION
AEEDDSDVWWGGADTDYAD

SEQ ID NO: 83 - protein, 18 aa, synthetic construct
  MOD_RES 1: note = ACETYLATION
  MOD_RES 18: note = AMIDATION
EEDDSDVWWGGADTDYAD

SEQ ID NO: 84 - protein, 19 aa, synthetic construct
  MOD_RES 1: note = ACETYLATION
  MOD_RES 19: note = AMIDATION
EEDDSDVWWGGADTDYADG

SEQ ID NO: 85 - protein, 20 aa, synthetic construct
  MOD_RES 1: note = ACETYLATION
  MOD_RES 20: note = AMIDATION
EEDDSDVWWGGADTDYADGG

SEQ ID NO: 86 - protein, 21 aa, synthetic construct
  MOD_RES 1: note = ACETYLATION
  MOD_RES 21: note = AMIDATION
EEDDSDVWWGGADTDYADGGE

SEQ ID NO: 87 - protein, 22 aa, synthetic construct
  MOD_RES 1: note = ACETYLATION
  MOD_RES 22: note = AMIDATION
EEDDSDVWWGGADTDYADGGED

SEQ ID NO: 88 - protein, 23 aa, synthetic construct
  MOD_RES 1: note = ACETYLATION
  MOD_RES 23: note = AMIDATION
EEDDSDVWWGGADTDYADGGEDK

SEQ ID NO: 89 - protein, 9 aa, synthetic construct
  MOD_RES 1: note = ACETYLATION
  MOD_RES 9: note = AMIDATION
GGADTDYAD

SEQ ID NO: 90 - protein, 10 aa, synthetic construct
  MOD_RES 1: note = ACETYLATION
  MOD_RES 10: note = AMIDATION
WGGADTDYAD

SEQ ID NO: 91 - protein, 11 aa, synthetic construct
  MOD_RES 1: note = ACETYLATION
  MOD_RES 11: note = AMIDATION
WWGGADTDYAD

SEQ ID NO: 92 - protein, 12 aa, synthetic construct
  MOD_RES 1: note = ACETYLATION
  MOD_RES 12: note = AMIDATION
VWWGGADTDYAD

SEQ ID NO: 93 - protein, 13 aa, synthetic construct
  MOD_RES 1: note = ACETYLATION
  MOD_RES 13: note = AMIDATION
DVWWGGADTDYAD

SEQ ID NO: 94 - protein, 14 aa, synthetic construct
  MOD_RES 1: note = ACETYLATION
  MOD_RES 14: note = AMIDATION
SDVWWGGADTDYAD

SEQ ID NO: 95 - protein, 15 aa, synthetic construct
  MOD_RES 1: note = ACETYLATION
  MOD_RES 15: note = AMIDATION
DSDVWWGGADTDYAD

SEQ ID NO: 96 - protein, 16 aa, synthetic construct
  MOD_RES 1: note = ACETYLATION
  MOD_RES 16: note = AMIDATION
DDSDVWWGGADTDYAD

SEQ ID NO: 97 - protein, 17 aa, synthetic construct
  MOD_RES 1: note = ACETYLATION
  MOD_RES 17: note = AMIDATION
EDDSDVWWGGADTDYAD

SEQ ID NO: 98 - protein, 18 aa, synthetic construct
  MOD_RES 1: note = ACETYLATION
  MOD_RES 18: note = AMIDATION
EEDDSDVWWGGADTDYAD

SEQ ID NO: 99 - protein, 17 aa, synthetic construct
  MOD_RES 1: note = ACETYLATION
  MOD_RES 17: note = AMIDATION
EEDDSDVWWGGADTDYA

SEQ ID NO: 100 - protein, 16 aa, synthetic construct
  MOD_RES 1: note = ACETYLATION
  MOD_RES 16: note = AMIDATION
EEDDSDVWWGGADTDY

SEQ ID NO: 101 - protein, 15 aa, synthetic construct
  MOD_RES 1: note = ACETYLATION
  MOD_RES 15: note = AMIDATION
EEDDSDVWWGGADTD

SEQ ID NO: 102 - protein, 14 aa, synthetic construct
  MOD_RES 1: note = ACETYLATION
  MOD_RES 14: note = AMIDATION
EEDDSDVWWGGADT

SEQ ID NO: 103 - protein, 13 aa, synthetic construct
  MOD_RES 1: note = ACETYLATION
  MOD_RES 13: note = AMIDATION
EEDDSDVWWGGAD

SEQ ID NO: 104 - protein, 12 aa, synthetic construct
  MOD_RES 1: note = ACETYLATION
  MOD_RES 12: note = AMIDATION
EEDDSDVWWGGA

SEQ ID NO: 105 - protein, 11 aa, synthetic construct
  MOD_RES 1: note = ACETYLATION
  MOD_RES 11: note = AMIDATION
EEDDSDVWWGG

SEQ ID NO: 106 - protein, 10 aa, synthetic construct
  MOD_RES 1: note = ACETYLATION
  MOD_RES 10: note = AMIDATION
EEDDSDVWWG

SEQ ID NO: 107 - protein, 9 aa, synthetic construct
  MOD_RES 1: note = ACETYLATION
  MOD_RES 9: note = AMIDATION
EEDDSDVWW

SEQ ID NO: 108 - protein, 8 aa, synthetic construct
  MOD_RES 1: note = ACETYLATION
  MOD_RES 8: note = AMIDATION
EEDDSDVW

SEQ ID NO: 109 - protein, 18 aa, synthetic construct
  MOD_RES 1: note = ACETYLATION
  MOD_RES 18: note = AMIDATION
RPTTETEFIAWGPTGDED

SEQ ID NO: 110 - protein, 17 aa, synthetic construct
  MOD_RES 1: note = ACETYLATION
  MOD_RES 17: note = AMIDATION
PTTETEFIAWGPTGDED

SEQ ID NO: 111 - protein, 16 aa, synthetic construct
  MOD_RES 1: note = ACETYLATION
  MOD_RES 16: note = AMIDATION
TTETEFIAWGPTGDED

SEQ ID NO: 112 - protein, 15 aa, synthetic construct
  MOD_RES 1: note = ACETYLATION
  MOD_RES 15: note = AMIDATION
TETEFIAWGPTGDED

SEQ ID NO: 113 - protein, 16 aa, synthetic construct
  MOD_RES 1: note = ACETYLATION
  MOD_RES 16: note = AMIDATION
TETEFIAWGPTGDEDA

SEQ ID NO: 114 - protein, 17 aa, synthetic construct
  MOD_RES 1: note = ACETYLATION
  MOD_RES 17: note = AMIDATION
TETEFIAWGPTGDEDAL

SEQ ID NO: 115 - protein, 9 aa, synthetic construct
  MOD_RES 1: note = ACETYLATION
  MOD_RES 9: note = AMIDATION
AWGPTGDED

SEQ ID NO: 116 - protein, 10 aa, synthetic construct
  MOD_RES 1: note = ACETYLATION
  MOD_RES 10: note = AMIDATION
IAWGPTGDED

SEQ ID NO: 117 - protein, 11 aa, synthetic construct
  MOD_RES 1: note = ACETYLATION
  MOD_RES 11: note = AMIDATION
FIAWGPTGDED

SEQ ID NO: 118 - protein, 12 aa, synthetic construct
  MOD_RES 1: note = ACETYLATION
  MOD_RES 12: note = AMIDATION
EFIAWGPTGDED

SEQ ID NO: 119 - protein, 13 aa, synthetic construct
  MOD_RES 1: note = ACETYLATION
  MOD_RES 13: note = AMIDATION
TEFIAWGPTGDED

SEQ ID NO: 120 - protein, 14 aa, synthetic construct
  MOD_RES 1: note = ACETYLATION
  MOD_RES 14: note = AMIDATION
ETEFIAWGPTGDED

SEQ ID NO: 121 - protein, 15 aa, synthetic construct
  MOD_RES 1: note = ACETYLATION
  MOD_RES 15: note = AMIDATION
TETEFIAWGPTGDED

SEQ ID NO: 122 - protein, 14 aa, synthetic construct
  MOD_RES 1: note = ACETYLATION
  MOD_RES 14: note = AMIDATION
TETEFIAWGPTGDE

SEQ ID NO: 123 - protein, 13 aa, synthetic construct
  MOD_RES 1: note = ACETYLATION
  MOD_RES 13: note = AMIDATION
TETEFIAWGPTGD

SEQ ID NO: 124 - protein, 12 aa, synthetic construct
  MOD_RES 1: note = ACETYLATION
  MOD_RES 12: note = AMIDATION
TETEFIAWGPTG

SEQ ID NO: 125 - protein, 11 aa, synthetic construct
  MOD_RES 1: note = ACETYLATION
  MOD_RES 11: note = AMIDATION
TETEFIAWGPT

SEQ ID NO: 126 - protein, 10 aa, synthetic construct
  MOD_RES 1: note = ACETYLATION
  MOD_RES 10: note = AMIDATION
TETEFIAWGP

SEQ ID NO: 127 - protein, 9 aa, synthetic construct
  MOD_RES 1: note = ACETYLATION
  MOD_RES 9: note = AMIDATION
TETEFIAWG

SEQ ID NO: 128 - protein, 8 aa, synthetic construct
  MOD_RES 1: note = ACETYLATION
  MOD_RES 8: note = AMIDATION
TETEFIAW

SEQ ID NO: 129 - protein, 20 aa, synthetic construct
PSSQYPWAIVWGPTVSREDG

SEQ ID NO: 130 - protein, 19 aa, synthetic construct
SSQYPWAIVWGPTVSREDG

SEQ ID NO: 131 - protein, 18 aa, synthetic construct
SQYPWAIVWGPTVSREDG

SEQ ID NO: 132 - protein, 17 aa, synthetic construct
QYPWAIVWGPTVSREDG

SEQ ID NO: 133 - protein, 16 aa, synthetic construct
YPWAIVWGPTVSREDG

SEQ ID NO: 134 - protein, 15 aa, synthetic construct
PWAIVWGPTVSREDG

SEQ ID NO: 135 - protein, 16 aa, synthetic construct
PWAIVWGPTVSREDGG

SEQ ID NO: 136 - protein, 17 aa, synthetic construct
PWAIVWGPTVSREDGGD

SEQ ID NO: 137 - protein, 18 aa, synthetic construct
PWAIVWGPTVSREDGGDP

SEQ ID NO: 138 - protein, 19 aa, synthetic construct
PWAIVWGPTVSREDGGDPN

SEQ ID NO: 139 - protein, 20 aa, synthetic construct
PWAIVWGPTVSREDGGDPNS

SEQ ID NO: 140 - protein, 11 aa, synthetic construct
VWGPTVSREDG

SEQ ID NO: 141 - protein, 12 aa, synthetic construct
IVWGPTVSREDG

SEQ ID NO: 142 - protein, 13 aa, synthetic construct
AIVWGPTVSREDG

SEQ ID NO: 143 - protein, 14 aa, synthetic construct
WAIVWGPTVSREDG

SEQ ID NO: 144 - protein, 15 aa, synthetic construct
PWAIVWGPTVSREDG

SEQ ID NO: 145 - protein, 14 aa, synthetic construct
PWAIVWGPTVSRED

SEQ ID NO: 146 - protein, 13 aa, synthetic construct
PWAIVWGPTVSRE

SEQ ID NO: 147 - protein, 12 aa, synthetic construct
PWAIVWGPTVSR

SEQ ID NO: 148 - protein, 11 aa, synthetic construct
PWAIVWGPTVS

SEQ ID NO: 149 - protein, 10 aa, synthetic construct
PWAIVWGPTV

SEQ ID NO: 150 - protein, 9 aa, synthetic construct
PWAIVWGPT

SEQ ID NO: 151 - protein, 8 aa, synthetic construct
PWAIVWGP

SEQ ID NO: 152 - protein, 7 aa, synthetic construct
PWAIVWG

SEQ ID NO: 153 - protein, 6 aa, synthetic construct
PWAIVW

SEQ ID NO: 154 - protein, 14 aa, synthetic construct
ADDSDVWWGGADTD

SEQ ID NO: 155 - protein, 14 aa, synthetic construct
EADSDVWWGGADTD

SEQ ID NO: 156 - protein, 14 aa, synthetic construct
EDASDVWWGGADTD

SEQ ID NO: 157 - protein, 14 aa, synthetic construct
EDDADVWWGGADTD

SEQ ID NO: 158 - protein, 14 aa, synthetic construct
EDDSAVWWGGADTD

SEQ ID NO: 159 - protein, 14 aa, synthetic construct
EDDSDAWWGGADTD

SEQ ID NO: 160 - protein, 14 aa, synthetic construct
EDDSDVAWGGADTD

SEQ ID NO: 161 - protein, 14 aa, synthetic construct
EDDSDVWAGGADTD

SEQ ID NO: 162 - protein, 14 aa, synthetic construct
EDDSDVWWAGADTD

SEQ ID NO: 163 - protein, 14 aa, synthetic construct
EDDSDVWWGAADTD

SEQ ID NO: 164 - protein, 14 aa, synthetic construct
EDDSDVWWGGAATD

SEQ ID NO: 165 - protein, 14 aa, synthetic construct
EDDSDVWWGGADAD

SEQ ID NO: 166 - protein, 14 aa, synthetic construct
EDDSDVWWGGADTA

SEQ ID NO: 167 - protein, 14 aa, synthetic construct
  SITE 1: note = D-GLUTAMIC ACID
EDDSDVWWGGADTD

SEQ ID NO: 168 - protein, 14 aa, synthetic construct
  SITE 2: note = D-ASPARTIC ACID
EDDSDVWWGGADTD

SEQ ID NO: 169 - protein, 14 aa, synthetic construct
  SITE 3: note = D-ASPARTIC ACID
EDDSDVWWGGADTD

SEQ ID NO: 170 - protein, 14 aa, synthetic construct
  SITE 4: note = D-SERINE
EDDSDVWWGGADTD

SEQ ID NO: 171 - protein, 14 aa, synthetic construct
  SITE 5: note = D-ASPARTIC ACID
EDDSDVWWGGADTD

SEQ ID NO: 172 - protein, 14 aa, synthetic construct
  SITE 6: note = D-VALINE
EDDSDVWWGGADTD

SEQ ID NO: 173 - protein, 14 aa, synthetic construct
  SITE 7: note = D-TRYPTOPHAN
EDDSDVWWGGADTD

SEQ ID NO: 174 - protein, 14 aa, synthetic construct
  SITE 8: note = D-TRYPTOPHAN
EDDSDVWWGGADTD

SEQ ID NO: 175 - protein, 14 aa, synthetic construct
  SITE 9: note = D-ALANINE
EDDSDVWWAGADTD

SEQ ID NO: 176 - protein, 14 aa, synthetic construct
  SITE 10: note = D-ALANINE
EDDSDVWWGAADTD

SEQ ID NO: 177 - protein, 14 aa, synthetic construct
  SITE 11: note = D-ALANINE
EDDSDVWWGGADTD

SEQ ID NO: 178 - protein, 14 aa, synthetic construct
  SITE 12: note = D-ASPARTIC ACID
EDDSDVWWGGADTD

SEQ ID NO: 179 - protein, 14 aa, synthetic construct
  SITE 13: note = D-THREONINE
EDDSDVWWGGADTD

SEQ ID NO: 180 - protein, 14 aa, synthetic construct
  SITE 14: note = D-ASPARTIC ACID
EDDSDVWWGGADTD

SEQ ID NO: 181 - protein, 14 aa, synthetic construct
  MOD_RES 1: note = N-METHYLATION
EDDSDVWWGGADTD

SEQ ID NO: 182 - protein, 14 aa, synthetic construct
  MOD_RES 2: note = N-METHYLATION
EDDSDVWWGGADTD

SEQ ID NO: 183 - protein, 14 aa, synthetic construct
  MOD_RES 3: note = N-METHYLATION
EDDSDVWWGGADTD

SEQ ID NO: 184 - protein, 14 aa, synthetic construct
  MOD_RES 4: note = N-METHYLATION
EDDSDVWWGGADTD

SEQ ID NO: 185 - protein, 14 aa, synthetic construct
  MOD_RES 5: note = N-METHYLATION
EDDSDVWWGGADTD

SEQ ID NO: 186 - protein, 14 aa, synthetic construct
  MOD_RES 6: note = N-METHYLATION
EDDSDVWWGGADTD

SEQ ID NO: 187 - protein, 14 aa, synthetic construct
  MOD_RES 7: note = N-METHYLATION
EDDSDVWWGGADTD

SEQ ID NO: 188 - protein, 14 aa, synthetic construct
  MOD_RES 8: note = N-METHYLATION
EDDSDVWWGGADTD

SEQ ID NO: 189 - protein, 14 aa, synthetic construct
  MOD_RES 9: note = MeGly
EDDSDVWWGGADTD

SEQ ID NO: 190 - protein, 14 aa, synthetic construct
  MOD_RES 10: note = MeGly
EDDSDVWWGGADTD

SEQ ID NO: 191 - protein, 14 aa, synthetic construct
  MOD_RES 11: note = N-METHYLATION
EDDSDVWWGGADTD

SEQ ID NO: 192 - protein, 14 aa, synthetic construct
  MOD_RES 12: note = N-METHYLATION
EDDSDVWWGGADTD

SEQ ID NO: 193 - protein, 14 aa, synthetic construct
  MOD_RES 13: note = N-METHYLATION
EDDSDVWWGGADTD

SEQ ID NO: 194 - protein, 14 aa, synthetic construct
  MOD_RES 14: note = N-METHYLATION
EDDSDVWWGGADTD

SEQ ID NO: 195 - protein, 14 aa, synthetic construct
ATEFIAWGPTGDED

SEQ ID NO: 196 - protein, 14 aa, synthetic construct
EAEFIAWGPTGDED

SEQ ID NO: 197 - protein, 14 aa, synthetic construct
ETAFIAWGPTGDED

SEQ ID NO: 198 - protein, 14 aa, synthetic construct
ETEAIAWGPTGDED

SEQ ID NO: 199 - protein, 14 aa, synthetic construct
ETEFAAWGPTGDED

SEQ ID NO: 200 - protein, 14 aa, synthetic construct
ETEFIAAGPTGDED

SEQ ID NO: 201 - protein, 14 aa, synthetic construct
ETEFIAWAPTGDED

SEQ ID NO: 202 - protein, 14 aa, synthetic construct
ETEFIAWGATGDED

SEQ ID NO: 203 - protein, 14 aa, synthetic construct
ETEFIAWGPAGDED

SEQ ID NO: 204 - protein, 14 aa, synthetic construct
ETEFIAWGPTADED

SEQ ID NO: 205 - protein, 14 aa, synthetic construct
ETEFIAWGPTGAED

SEQ ID NO: 206 - protein, 14 aa, synthetic construct
ETEFIAWGPTGDAD

SEQ ID NO: 207 - protein, 14 aa, synthetic construct
ETEFIAWGPTGDEA

SEQ ID NO: 208 - protein, 14 aa, synthetic construct
  SITE 1: note = D-GLUTAMIC ACID
ETEFIAWGPTGDED

SEQ ID NO: 209 - protein, 14 aa, synthetic construct
  SITE 2: note = D-THREONINE
ETEFIAWGPTGDED

SEQ ID NO: 210 - protein, 14 aa, synthetic construct
  SITE 3: note = D-GLUTAMIC ACID
ETEFIAWGPTGDED

SEQ ID NO: 211 - protein, 14 aa, synthetic construct
  SITE 4: note = D-PHENYLALANINE
ETEFIAWGPTGDED

SEQ ID NO: 212 - protein, 14 aa, synthetic construct
  SITE 5: note = D-ISOLEUCINE
ETEFIAWGPTGDED

SEQ ID NO: 213 - protein, 14 aa, synthetic construct
  SITE 6: note = D-ALANINE
ETEFIAWGPTGDED

SEQ ID NO: 214 - protein, 14 aa, synthetic construct
  SITE 7: note = D-TRYPTOPHAN
ETEFIAWGPTGDED

SEQ ID NO: 215 - protein, 14 aa, synthetic construct
  SITE 8: note = D-ALANINE
ETEFIAWAPTGDED

SEQ ID NO: 216 - protein, 14 aa, synthetic construct
  SITE 9: note = D-PROLINE
ETEFIAWGPTGDED

SEQ ID NO: 217 - protein, 14 aa, synthetic construct
  SITE 10: note = D-THREONINE
ETEFIAWGPTGDED

SEQ ID NO: 218 - protein, 14 aa, synthetic construct
  SITE 11: note = D-ALANINE
ETEFIAWGPTADED

SEQ ID NO: 219 - protein, 14 aa, synthetic construct
  SITE 12: note = D-ASPARTIC ACID
ETEFIAWGPTGDED

SEQ ID NO: 220 - protein, 14 aa, synthetic construct
  SITE 13: note = D-GLUTAMIC ACID
ETEFIAWGPTGDED

SEQ ID NO: 221 - protein, 14 aa, synthetic construct
  SITE 14: note = D-ASPARTIC ACID
ETEFIAWGPTGDED

SEQ ID NO: 222 - protein, 14 aa, synthetic construct
  MOD_RES 1: note = N-METHYLATION
ETEFIAWGPTGDED

SEQ ID NO: 223 - protein, 14 aa, synthetic construct
  MOD_RES 2: note = N-METHYLATION
ETEFIAWGPTGDED

SEQ ID NO: 224 - protein, 14 aa, synthetic construct
  MOD_RES 3: note = N-METHYLATION
ETEFIAWGPTGDED

SEQ ID NO: 225 - protein, 14 aa, synthetic construct
  MOD_RES 4: note = N-METHYLATION
ETEFIAWGPTGDED

SEQ ID NO: 226 - protein, 14 aa, synthetic construct
  MOD_RES 5: note = N-METHYLATION
ETEFIAWGPTGDED

SEQ ID NO: 227 - protein, 14 aa, synthetic construct
  MOD_RES 6: note = N-METHYLATION
ETEFIAWGPTGDED

SEQ ID NO: 228 - protein, 14 aa, synthetic construct
  MOD_RES 7: note = N-METHYLATION
ETEFIAWGPTGDED

SEQ ID NO: 229 - protein, 14 aa, synthetic construct
  MOD_RES 8: note = MeGly
ETEFIAWGPTGDED

SEQ ID NO: 230 - protein, 14 aa, synthetic construct
  MOD_RES 9: note = MeGly
ETEFIAWGGTGDED

SEQ ID NO: 231 - protein, 14 aa, synthetic construct
  MOD_RES 10: note = N-METHYLATION
ETEFIAWGPTGDED

SEQ ID NO: 232 - protein, 14 aa, synthetic construct
  MOD_RES 11: note = MeGly
ETEFIAWGPTGDED

SEQ ID NO: 233 - protein, 14 aa, synthetic construct
  MOD_RES 12: note = N-METHYLATION
ETEFIAWGPTGDED

SEQ ID NO: 234 - protein, 14 aa, synthetic construct
  MOD_RES 13: note = N-METHYLATION
ETEFIAWGPTGDED

SEQ ID NO: 235 - protein, 14 aa, synthetic construct
  MOD_RES 14: note = N-METHYLATION
ETEFIAWGPTGDED

SEQ ID NO: 236 - protein, 14 aa, synthetic construct
AWAIVWGPTVSRED

SEQ ID NO: 237 - protein, 14 aa, synthetic construct
PAAIVWGPTVSRED

SEQ ID NO: 238 - protein, 14 aa, synthetic construct
PWAAVWGPTVSRED

SEQ ID NO: 239 - protein, 14 aa, synthetic construct
PWAIAWGPTVSRED

SEQ ID NO: 240 - protein, 14 aa, synthetic construct
PWAIVAGPTVSRED

SEQ ID NO: 241 - protein, 14 aa, synthetic construct
PWAIVWAPTVSRED

SEQ ID NO: 242 - protein, 14 aa, synthetic construct
PWAIVWGATVSRED

SEQ ID NO: 243 - protein, 14 aa, synthetic construct
PWAIVWGPAVSRED

SEQ ID NO: 244 - protein, 14 aa, synthetic construct
PWAIVWGPTASRED

SEQ ID NO: 245 - protein, 14 aa, synthetic construct
PWAIVWGPTVARED

SEQ ID NO: 246 - protein, 14 aa, synthetic construct
PWAIVWGPTVSAED

SEQ ID NO: 247 - protein, 14 aa, synthetic construct
PWAIVWGPTVSRAD

SEQ ID NO: 248 - protein, 14 aa, synthetic construct
PWAIVWGPTVSREA

SEQ ID NO: 249 - protein, 14 aa, synthetic construct
  SITE 1: note = D-PROLINE
PWAIVWGPTVSRED

SEQ ID NO: 250 - protein, 14 aa, synthetic construct
  SITE 2: note = D-TRYPTOPHAN
PWAIVWGPTVSRED

SEQ ID NO: 251 - protein, 14 aa, synthetic construct
  SITE 3: note = D-ALANINE
PWAIVWGPTVSRED

SEQ ID NO: 252 - protein, 14 aa, synthetic construct
  SITE 4: note = D-ISOLEUCINE
PWAIVWGPTVSRED

SEQ ID NO: 253 - protein, 14 aa, synthetic construct
  SITE 5: note = D-VALINE
PWAIVWGPTVSRED

SEQ ID NO: 254 - protein, 14 aa, synthetic construct
  SITE 6: note = D-TRYPTOPHAN
PWAIVWGPTVSRED

SEQ ID NO: 255 - protein, 14 aa, synthetic construct
  MOD_RES 7: note = D-ALANINE
PWAIVWAPTVSRED

SEQ ID NO: 256 - protein, 14 aa, synthetic construct
  SITE 8: note = D-PROLINE
PWAIVWGPTVSRED

SEQ ID NO: 257 - protein, 14 aa, synthetic construct
  SITE 9: note = D-THREONINE
PWAIVWGPTVSRED

SEQ ID NO: 258 - protein, 14 aa, synthetic construct
  SITE 10: note = D-VALINE
PWAIVWGPTVSRED

SEQ ID NO: 259 - protein, 14 aa, synthetic construct
  SITE 11: note = D-SERINE
PWAIVWGPTVSRED

SEQ ID NO: 260 - protein, 14 aa, synthetic construct
  SITE 12: note = D-ARGININE
PWAIVWGPTVSRED

SEQ ID NO: 261 - protein, 14 aa, synthetic construct
  SITE 13: note = D-GLUTAMIC ACID
PWAIVWGPTVSRED

SEQ ID NO: 262 - protein, 14 aa, synthetic construct
  SITE 14: note = D-ASPARTIC ACID
PWAIVWGPTVSRED

SEQ ID NO: 263 - protein, 14 aa, synthetic construct
  MOD_RES 2: note = N-METHYLATION
PWAIVWGPTVSRED

SEQ ID NO: 264 - protein, 14 aa, synthetic construct
  MOD_RES 3: note = N-METHYLATION
PWAIVWGPTVSRED

SEQ ID NO: 265 - protein, 14 aa, synthetic construct
  MOD_RES 4: note = N-METHYLATION
PWAIVWGPTVSRED

SEQ ID NO: 266 - protein, 14 aa, synthetic construct
  MOD_RES 5: note = N-METHYLATION
PWAIVWGPTVSRED

SEQ ID NO: 267 - protein, 14 aa, synthetic construct
  MOD_RES 6: note = N-METHYLATION
PWAIVWGPTVSRED

SEQ ID NO: 268 - protein, 14 aa, synthetic construct
  MOD_RES 7: note = MeGly
PWAIVWGPTVSRED

SEQ ID NO: 269 - protein, 14 aa, synthetic construct
  MOD_RES 8: note = MeGly
PWAIVWGGTVSRED

SEQ ID NO: 270 - protein, 14 aa, synthetic construct
  MOD_RES 9: note = N-METHYLATION
PWAIVWGPTVSRED

SEQ ID NO: 271 - protein, 14 aa, synthetic construct
  MOD_RES 10: note = N-METHYLATION
PWAIVWGPTVSRED

SEQ ID NO: 272 - protein, 14 aa, synthetic construct
  MOD_RES 11: note = N-METHYLATION
PWAIVWGPTVSRED

SEQ ID NO: 273 - protein, 14 aa, synthetic construct
  MOD_RES 12: note = N-METHYLATION
PWAIVWGPTVSRED

SEQ ID NO: 274 - protein, 14 aa, synthetic construct
  MOD_RES 13: note = N-METHYLATION
PWAIVWGPTVSRED

SEQ ID NO: 275 - protein, 14 aa, synthetic construct
  MOD_RES 14: note = N-METHYLATION
PWAIVWGPTVSRED

SEQ ID NO: 276 - protein, 14 aa, synthetic construct
KDDSDVWWGGADTD

SEQ ID NO: 277 - protein, 14 aa, synthetic construct
EDDWDVWWGGADTD

SEQ ID NO: 278 - protein, 14 aa, synthetic construct
EDDSFVWWGGADTD

SEQ ID NO: 279 - protein, 14 aa, synthetic construct
EDDSIVWWGGADTD

SEQ ID NO: 280 - protein, 14 aa, synthetic construct
EDDSYVWWGGADTD

SEQ ID NO: 281 - protein, 14 aa, synthetic construct
EDDSWVWWGGADTD

SEQ ID NO: 282 - protein, 14 aa, synthetic construct
EDDSDIWWGGADTD

SEQ ID NO: 283 - protein, 14 aa, synthetic construct
EDDSDVDWGGADTD

SEQ ID NO: 284 - protein, 14 aa, synthetic construct
EDDSDVIWGGADTD

SEQ ID NO: 285 - protein, 14 aa, synthetic construct
EDDSDVYWGGADTD

SEQ ID NO: 286 - protein, 14 aa, synthetic construct
EDDSDVWWDGADTD

SEQ ID NO: 287 - protein, 14 aa, synthetic construct
EDDSDVWWHGADTD

SEQ ID NO: 288 - protein, 14 aa, synthetic construct
EDDSDVWWYGADTD

SEQ ID NO: 289 - protein, 14 aa, synthetic construct
EDDSDVWWGPADTD

SEQ ID NO: 290 - protein, 14 aa, synthetic construct
EDDSDVWWGGHDTD

SEQ ID NO: 291 - protein, 14 aa, synthetic construct
EDDSDVWWGGSDTD

SEQ ID NO: 292 - protein, 14 aa, synthetic construct
EDDSDVWWGGYDTD

SEQ ID NO: 293 - protein, 14 aa, synthetic construct
EDDSDVWWGGADED

SEQ ID NO: 294 - protein, 14 aa, synthetic construct
EDDSDVWWGGADHD

SEQ ID NO: 295 - protein, 14 aa, synthetic construct
EDDSDVWWGGADWD

SEQ ID NO: 296 - protein, 14 aa, synthetic construct
RTEFIAWGPTGDED

SEQ ID NO: 297 - protein, 14 aa, synthetic construct
NTEFIAWGPTGDED

SEQ ID NO: 298 - protein, 14 aa, synthetic construct
VTEFIAWGPTGDED

SEQ ID NO: 299 - protein, 14 aa, synthetic construct
EIEFIAWGPTGDED

SEQ ID NO: 300 - protein, 14 aa, synthetic construct
EVEFIAWGPTGDED

SEQ ID NO: 301 - protein, 14 aa, synthetic construct
ETDFIAWGPTGDED

SEQ ID NO: 302 - protein, 14 aa, synthetic construct
ETHFIAWGPTGDED

SEQ ID NO: 303 - protein, 14 aa, synthetic construct
ETSFIAWGPTGDED

SEQ ID NO: 304 - protein, 14 aa, synthetic construct
ETEWIAWGPTGDED

SEQ ID NO: 305 - protein, 14 aa, synthetic construct
ETEYIAWGPTGDED

SEQ ID NO: 306 - protein, 14 aa, synthetic construct
ETEKIAWGPTGDED

SEQ ID NO: 307 - protein, 14 aa, synthetic construct
ETEFIPWGPTGDED

SEQ ID NO: 308 - protein, 14 aa, synthetic construct
ETEFIDWGPTGDED

SEQ ID NO: 309 - protein, 14 aa, synthetic construct
ETEFIHWGPTGDED

SEQ ID NO: 310 - protein, 14 aa, synthetic construct
ETEFIVWGPTGDED

SEQ ID NO: 311 - protein, 14 aa, synthetic construct
ETEFIAWHPTGDED

SEQ ID NO: 312 - protein, 14 aa, synthetic construct
ETEFIAWYPTGDED

SEQ ID NO: 313 - protein, 14 aa, synthetic construct
ETEFIAWGPFGDED

SEQ ID NO: 314 - protein, 14 aa, synthetic construct
ETEFIAWGPHGDED

SEQ ID NO: 315 - protein, 14 aa, synthetic construct
ETEFIAWGPSGDED

SEQ ID NO: 316 - protein, 14 aa, synthetic construct
ETEFIAWGPTFDED

SEQ ID NO: 317 - protein, 14 aa, synthetic construct
ETEFIAWGPTHDED

SEQ ID NO: 318 - protein, 14 aa, synthetic construct
ETEFIAWGPTRDED

SEQ ID NO: 319 - protein, 14 aa, synthetic construct
ETEFIAWGPTGHED

SEQ ID NO: 320 - protein, 14 aa, synthetic construct
ETEFIAWGPTGYED

SEQ ID NO: 321 - protein, 14 aa, synthetic construct
ETEFIAWGPTGDDD

SEQ ID NO: 322 - protein, 14 aa, synthetic construct
ETEFIAWGPTGDFD

SEQ ID NO: 323 - protein, 14 aa, synthetic construct
ETEFIAWGPTGDHD

SEQ ID NO: 324 - protein, 14 aa, synthetic construct
ETEFIAWGPTGDND

SEQ ID NO: 325 - protein, 14 aa, synthetic construct
ETEFIAWGPTGDPD

SEQ ID NO: 326 - protein, 14 aa, synthetic construct
ETEFIAWGPTGDRD

SEQ ID NO: 327 - protein, 14 aa, synthetic construct
ETEFIAWGPTGDVD

SEQ ID NO: 328 - protein, 14 aa, synthetic construct
ETEFIAWGPTGDWD

SEQ ID NO: 329 - protein, 14 aa, synthetic construct
ETEFIAWGPTGDYD

SEQ ID NO: 330 - protein, 14 aa, synthetic construct
ETEFIAWGPTGDEF

SEQ ID NO: 331 - protein, 14 aa, synthetic construct
ETEFIAWGPTGDEH

SEQ ID NO: 332 - protein, 14 aa, synthetic construct
ETEFIAWGPTGDEN

SEQ ID NO: 333 - protein, 14 aa, synthetic construct
ETEFIAWGPTGDEP

SEQ ID NO: 334 - protein, 14 aa, synthetic construct
ETEFIAWGPTGDEQ

SEQ ID NO: 335 - protein, 14 aa, synthetic construct
ETEFIAWGPTGDEV

SEQ ID NO: 336 - protein, 14 aa, synthetic construct
ETEFIAWGPTGDEW

SEQ ID NO: 337 - protein, 14 aa, synthetic construct
ETEFIAWGPTGDEY

SEQ ID NO: 338 - protein, 14 aa, synthetic construct
VWAIVWGPTVSRED

SEQ ID NO: 339 - protein, 14 aa, synthetic construct
DWAIVWGPTVSRED

SEQ ID NO: 340 - protein, 14 aa, synthetic construct
PDAIVWGPTVSRED

SEQ ID NO: 341 - protein, 13 aa, synthetic construct
DAIVWGPTVSRED

SEQ ID NO: 342 - protein, 13 aa, synthetic construct
DAIVWGPTVSRED

SEQ ID NO: 343 - protein, 14 aa, synthetic construct
PHAIVWGPTVSRED

SEQ ID NO: 344 - protein, 14 aa, synthetic construct
PSAIVWGPTVSRED

SEQ ID NO: 345 - protein, 14 aa, synthetic construct
PFAIVWGPTVSRED

SEQ ID NO: 346 - protein, 14 aa, synthetic construct
PWFIVWGPTVSRED

SEQ ID NO: 347 - protein, 14 aa, synthetic construct
PWIIVWGPTVSRED

SEQ ID NO: 348 - protein, 14 aa, synthetic construct
PWVIVWGPTVSRED

SEQ ID NO: 349 - protein, 14 aa, synthetic construct
PWYIVWGPTVSRED

SEQ ID NO: 350 - protein, 14 aa, synthetic construct
PWFIVWGPTVSDED

SEQ ID NO: 351 - protein, 14 aa, synthetic construct
PWAIDWGPTVSRED

SEQ ID NO: 352 - protein, 14 aa, synthetic construct
PWAIVWGPTSSRED

SEQ ID NO: 353 - protein, 14 aa, synthetic construct
PWAIVWGPTDSRED

SEQ ID NO: 354 - protein, 14 aa, synthetic construct
PWAIVWGPTVDRED

SEQ ID NO: 355 - protein, 14 aa, synthetic construct
PWAIVWGPTVSDED

SEQ ID NO: 356 - protein, 12 aa, synthetic construct
AIVWGPTVSRED

SEQ ID NO: 357 - protein, 12 aa, synthetic construct
AIVWGPTVSRED

SEQ ID NO: 358 - protein, 12 aa, synthetic construct
FIVWGPTVSRED

SEQ ID NO: 359 - protein, 12 aa, synthetic construct
FIVWGPTVSRED

SEQ ID NO: 360 - protein, 15 aa, synthetic construct
ETEFIAWGPTVSRED

SEQ ID NO: 361 - protein, 13 aa, synthetic construct
PWAIVWGPTGDED

SEQ ID NO: 362 - protein, 14 aa, synthetic construct
  MOD_RES 8: note = MeGly
PWAIVWGGTVSRED

SEQ ID NO: 363 - protein, 14 aa, synthetic construct
  MOD_RES 10: note = MeGly
EDDSWVWWGGADTD

SEQ ID NO: 364 - protein, 15 aa, synthetic construct
EDDSDVWWGPTGDED

SEQ ID NO: 365 - protein, 13 aa, synthetic construct
ETEFIAWGGADTD

SEQ ID NO: 366 - protein, 13 aa, synthetic construct
ETEFIAWYGADTD

SEQ ID NO: 367 - protein, 13 aa, synthetic construct
ETEFIAWYPADTD

SEQ ID NO: 368 - protein, 14 aa, synthetic construct
ETEFIAWYGPADTD

SEQ ID NO: 369 - protein, 14 aa, synthetic construct
ETEFIAWYPTGDED

SEQ ID NO: 370 - protein, 15 aa, synthetic construct
ETEFIAWYGPTGDED

SEQ ID NO: 371 - protein, 14 aa, synthetic construct
  MOD_RES 9: note = MeGly
ETEFIAWGGTGDED

SEQ ID NO: 372 - protein, 14 aa, synthetic construct
  MOD_RES 10: note = MeGly
EDDSDVWWGGADTD

SEQ ID NO: 373 - protein, 14 aa, synthetic construct
  MOD_RES 10: note = MeGly
EDDSDVWWYGADTD

SEQ ID NO: 374 - protein, 15 aa, synthetic construct
  MOD_RES 11: note = MeGly
EDDSDVWWYGGADTD

SEQ ID NO: 375 - protein, 15 aa, synthetic construct
  MOD_RES 10: note = MeGly
EDDSDVWWYGTGDED

SEQ ID NO: 376 - protein, 16 aa, synthetic construct
  MOD_RES 11: note = MeGly
EDDSDVWWYGGTGDED

SEQ ID NO: 377 - protein, 14 aa, synthetic construct
EDDSWIWWYGADTD

SEQ ID NO: 378 - protein, 15 aa, synthetic construct
EDDSWIWWGPTGDED

SEQ ID NO: 379 - protein, 14 aa, synthetic construct
EDDSWIDWYGADTD

SEQ ID NO: 380 - protein, 15 aa, synthetic construct
EDDSWIDWGPTGDED

SEQ ID NO: 381 - protein, 14 aa, synthetic construct
EDDDWIDWYDSDDD

SEQ ID NO: 382 - protein, 15 aa, synthetic construct
EDDDWIDWGPTSDDD

SEQ ID NO: 383 - protein, 13 aa, synthetic construct
EDDYIDWYDSDDD

SEQ ID NO: 384 - protein, 14 aa, synthetic construct
EDDYIDWGPTSDDD

SEQ ID NO: 385 - protein, 22 aa, synthetic construct
RRRRRRRREDDDWIDWYDSDDD

SEQ ID NO: 386 - protein, 22 aa, synthetic construct
EDDDWIDWYDSDDDRRRRRRRR

SEQ ID NO: 387 - protein, 25 aa, synthetic construct
YGRKKRRQRRREDDDWIDWYDSDDD

SEQ ID NO: 388 - protein, 25 aa, synthetic construct
EDDDWIDWYDSDDDYGRKKRRQRRR

SEQ ID NO: 389 - protein, 23 aa, synthetic construct
RRRRRRRREDDDWIDWGPTSDDD

SEQ ID NO: 390 - protein, 23 aa, synthetic construct
EDDDWIDWGPTSDDDRRRRRRRR

SEQ ID NO: 391 - protein, 26 aa, synthetic construct
YGRKKRRQRRREDDDWIDWGPTSDDD

SEQ ID NO: 392 - protein, 26 aa, synthetic construct
EDDDWIDWGPTSDDDYGRKKRRQRRR

SEQ ID NO: 393 - protein, 27 aa, synthetic construct
RRRRRRRREAAAKEDDDWIDWYDSDDD

SEQ ID NO: 394 - protein, 27 aa, synthetic construct
RRRRRRRRGGGGSEDDDWIDWYDSDDD

SEQ ID NO: 395 - protein, 28 aa, synthetic construct
RRRRRRRREAAAKEDDDWIDWGPTSDDD

SEQ ID NO: 396 - protein, 28 aa, synthetic construct
RRRRRRRRGGGGSEDDDWIDWGPTSDDD

SEQ ID NO: 397 - protein, 28 aa, synthetic construct
EDDDWIDWGPTSDDDEAAAKRRRRRRRR

SEQ ID NO: 398 - protein, 28 aa, synthetic construct
EDDDWIDWGPTSDDDGGGGSRRRRRRRR

SEQ ID NO: 399 - protein, 27 aa, synthetic construct
EDDDWIDWYDSDDDEAAAKRRRRRRRR

SEQ ID NO: 400 - protein, 27 aa, synthetic construct
EDDDWIDWYDSDDDGGGGSRRRRRRRR

SEQ ID NO: 401 - protein, 22 aa, synthetic construct
RRRRRRRREDDDWIDWYDSDDD

SEQ ID NO: 402 - protein, 21 aa, synthetic construct
RRRRRRREDDDWIDWYDSDDD

SEQ ID NO: 403 - protein, 20 aa, synthetic construct
RRRRRREDDDWIDWYDSDDD

SEQ ID NO: 404 - protein, 19 aa, synthetic construct
RRRRREDDDWIDWYDSDDD

SEQ ID NO: 405 - protein, 18 aa, synthetic construct
RRRREDDDWIDWYDSDDD

SEQ ID NO: 406 - protein, 17 aa, synthetic construct
RRREDDDWIDWYDSDDD

SEQ ID NO: 407 - protein, 23 aa, synthetic construct
RRRRRRRREDDDWIDWGPTSDDD

SEQ ID NO: 408 - protein, 22 aa, synthetic construct
RRRRRRREDDDWIDWGPTSDDD

SEQ ID NO: 409 - protein, 21 aa, synthetic construct
RRRRRREDDDWIDWGPTSDDD

SEQ ID NO: 410 - protein, 20 aa, synthetic construct
RRRRREDDDWIDWGPTSDDD

SEQ ID NO: 411 - protein, 19 aa, synthetic construct
RRRREDDDWIDWGPTSDDD

SEQ ID NO: 412 - protein, 18 aa, synthetic construct
RRREDDDWIDWGPTSDDD

SEQ ID NO: 413 - protein, 18 aa, synthetic construct
  SITE 9: note = 3-(2-NAPHTHYL)-L-ALANINE
RRRREDDDAIDWYDSDDD

SEQ ID NO: 414 - protein, 19 aa, synthetic construct
  SITE 9: note = 3-(2-NAPHTHYL)-L-ALANINE
RRRREDDDAIDWGPTSDDD

SEQ ID NO: 415 - protein, 18 aa, synthetic construct
  SITE 5: note = 3-(2-NAPHTHYL)-L-ALANINE
RRRRAEDSDDYDDIDDDW

SEQ ID NO: 416 - protein, 14 aa, synthetic construct
ETEFIAWGPTGDED

SEQ ID NO: 417 - protein, 14 aa, synthetic construct
EDDDWIDWYDSDDD

SEQ ID NO: 418 - protein, 15 aa, synthetic construct
EDDDWIDWGPTSDDD

SEQ ID NO: 419 - protein, 14 aa, synthetic construct
  SITE 5: note = 3-(2-NAPHTHYL)-L-ALANINE
EDDDAIDWYDSDDD

SEQ ID NO: 420 - protein, 15 aa, synthetic construct
  SITE 5: note = 3-(2-NAPHTHYL)-L-ALANINE
EDDDAIDWGPTSDDD

SEQ ID NO: 421 - protein, 14 aa, synthetic construct
  SITE 1: note = 3-(2-NAPHTHYL)-L-ALANINE
AEDSDDYDDIDDDW

SEQ ID NO: 422 - protein, 18 aa, synthetic construct
  SITE 5: note = 3-(2-NAPHTHYL)-L-ALANINE
RRRRAEDSDDYDDIDDDW

SEQ ID NO: 423 - protein, 18 aa, synthetic construct
  SITE 9: note = 3-(2-NAPHTHYL)-L-ALANINE
RRRREDDDAIDWYDSDDD

SEQ ID NO: 424 - protein, 19 aa, synthetic construct
  SITE 9: note = 3-(2-NAPHTHYL)-L-ALANINE
RRRREDDDAIDWGPTSDDD

SEQ ID NO: 425 - protein, 23 aa, synthetic construct
RRRRRRRREDDDWIDWGPTSDDD

SEQ ID NO: 426 - protein, 22 aa, synthetic construct
RRRRRRRREDDDWIDWYDSDDD

SEQ ID NO: 427 - protein, 23 aa, synthetic construct
EDDDWIDWGPTSDDDRRRRRRRR

SEQ ID NO: 428 - protein, 22 aa, synthetic construct
EDDDWIDWYDSDDDRRRRRRRR

SEQ ID NO: 429 - protein, 26 aa, synthetic construct
YGRKKRRQRRREDDDWIDWGPTSDDD

SEQ ID NO: 430 - protein, 25 aa, synthetic construct
YGRKKRRQRRREDDDWIDWYDSDDD

SEQ ID NO: 431 - protein, 25 aa, synthetic construct
EDDDWIDWYDSDDDYGRKKRRQRRR

SEQ ID NO: 432 - protein, 26 aa, synthetic construct
EDDDWIDWGPTSDDDYGRKKRRQRRR

SEQ ID NO: 433 - protein, 27 aa, synthetic construct
EDDDWIDWYDSDDDEAAAKRRRRRRRR

SEQ ID NO: 434 - protein, 27 aa, synthetic construct
EDDDWIDWYDSDDDGGGGSRRRRRRRR

SEQ ID NO: 435 - protein, 22 aa, synthetic construct
RRRRRRRREDDDWIDWYDSDDD

SEQ ID NO: 436 - protein, 21 aa, synthetic construct
RRRRRRREDDDWIDWYDSDDD

SEQ ID NO: 437 - protein, 20 aa, synthetic construct
RRRRRREDDDWIDWYDSDDD

SEQ ID NO: 438 - protein, 19 aa, synthetic construct
RRRRREDDDWIDWYDSDDD

SEQ ID NO: 439 - protein, 18 aa, synthetic construct
RRRREDDDWIDWYDSDDD

SEQ ID NO: 440 - protein, 17 aa, synthetic construct
RRREDDDWIDWYDSDDD

SEQ ID NO: 441 - protein, 23 aa, synthetic construct
RRRRRRRREDDDWIDWGPTSDDD

SEQ ID NO: 442 - protein, 22 aa, synthetic construct
RRRRRRREDDDWIDWGPTSDDD

SEQ ID NO: 443 - protein, 21 aa, synthetic construct
RRRRRREDDDWIDWGPTSDDD

SEQ ID NO: 444 - protein, 20 aa, synthetic construct
RRRRREDDDWIDWGPTSDDD

SEQ ID NO: 445 - protein, 19 aa, synthetic construct
RRRREDDDWIDWGPTSDDD

SEQ ID NO: 446 - protein, 18 aa, synthetic construct
RRREDDDWIDWGPTSDDD

SEQ ID NO: 447 - protein, 14 aa, synthetic construct
  SITE 5: note = 3-(1-NAPHTHYL)-L-ALANINE
EDDSAVWWGGADTD

SEQ ID NO: 448 - protein, 14 aa, synthetic construct
  SITE 5: note = 3-(2-NAPHTHYL)-L-ALANINE
EDDSAVWWGGADTD

SEQ ID NO: 449 - protein, 14 aa, synthetic construct
  SITE 5: note = O-BENZYL-L-TYROSINE
EDDSYVWWGGADTD

SEQ ID NO: 450 - protein, 14 aa, synthetic construct
  SITE 5: note = O-ETHYL-L-TYROSINE
EDDSYVWWGGADTD

SEQ ID NO: 451 - protein, 14 aa, synthetic construct
  SITE 5: note = O-METHYL-L-TYROSINE
EDDSYVWWGGADTD

SEQ ID NO: 452 - protein, 14 aa, synthetic construct
  SITE 5: note = p-fluorophenylalanine
EDDSFVWWGGADTD

SEQ ID NO: 453 - protein, 14 aa, synthetic construct
  SITE 5: note = p-aminophenylalanine
EDDSFVWWGGADTD

SEQ ID NO: 454 - protein, 14 aa, synthetic construct
  SITE 5: note = 4-tert-Butyl-l-phenylalanine
EDDSFVWWGGADTD

SEQ ID NO: 455 - protein, 14 aa, synthetic construct
  SITE 5: note = 3-(3-pyridyl)-L-alanine
EDDSAVWWGGADTD

SEQ ID NO: 456 - protein, 14 aa, synthetic construct
  SITE 5: note = 3-(4 pyridyl)-L-alanine
EDDSAVWWGGADTD

SEQ ID NO: 457 - protein, 14 aa, synthetic construct
  SITE 9: note = 3-(1-NAPHTHYL)-L-ALANINE
EDDSDVWWAGADTD

SEQ ID NO: 458 - protein, 14 aa, synthetic construct
  SITE 9: note = 3-(2-NAPHTHYL)-L-ALANINE
EDDSDVWWAGADTD

SEQ ID NO: 459 - protein, 14 aa, synthetic construct
  SITE 9: note = O-BENZYL-L-TYROSINE
EDDSDVWWYGADTD

SEQ ID NO: 460 - protein, 14 aa, synthetic construct
  SITE 9: note = O-ETHYL-L-TYROSINE
EDDSDVWWYGADTD

SEQ ID NO: 461 - protein, 14 aa, synthetic construct
  SITE 9: note = O-METHYL-L-TYROSINE
EDDSDVWWYGADTD

SEQ ID NO: 462 - protein, 14 aa, synthetic construct
  SITE 9: note = p-aminophenylalanine
EDDSDVWWFGADTD

SEQ ID NO: 463 - protein, 14 aa, synthetic construct
  SITE 8: note = 3-(1-NAPHTHYL)-L-ALANINE
EDDSDVWAGGADTD

SEQ ID NO: 464 - protein, 14 aa, synthetic construct
  SITE 8: note = 3-(2-NAPHTHYL)-L-ALANINE
EDDSDVWAGGADTD

SEQ ID NO: 465 - protein, 14 aa, synthetic construct
  SITE 8: note = O-BENZYL-L-TYROSINE
EDDSDVWYGGADTD

SEQ ID NO: 466 - protein, 14 aa, synthetic construct
  SITE 6: note = L-CYSTEIC ACID
ETEFICWGPTGDED

SEQ ID NO: 467 - protein, 14 aa, synthetic construct
  SITE 1: note = 3-(2-NAPHTHYL)-L-ALANINE
AEDSDDYDDIDDDW

SEQ ID NO: 468 - protein, 14 aa, synthetic construct
  SITE 5: note = 3-(2-NAPHTHYL)-L-ALANINE
EDDDAIDWYDSDDD

SEQ ID NO: 469 - protein, 15 aa, synthetic construct
  SITE 5: note = 3-(2-NAPHTHYL)-L-ALANINE
EDDDAIDWGPTSDDD

SEQ ID NO: 470 - protein, 14 aa, synthetic construct
  SITE 5: note = O-BENZYL-L-TYROSINE
EDDDYIDWYDSDDD

SEQ ID NO: 471 - protein, 15 aa, synthetic construct
  SITE 5: note = O-BENZYL-L-TYROSINE
EDDDYIDWGPTSDDD

SEQ ID NO: 472 - protein, 14 aa, synthetic construct
  SITE 5: note = O-ETHYL-L-TYROSINE
EDDDYIDWYDSDDD

SEQ ID NO: 473 - protein, 15 aa, synthetic construct
  SITE 5: note = O-ETHYL-L-TYROSINE
EDDDYIDWGPTSDDD

SEQ ID NO: 474 - protein, 5 aa, synthetic construct
RRRRR

SEQ ID NO: 475 - protein, 17 aa, synthetic construct
  SITE 1: note = ACETYLATION
  SITE 17: note = AMIDATION
  CROSSLNK 1..17: note = two cysteines linked via a non-peptide chemical linker, wherein the non-peptide chemical linker is a o-xylylene linker
CEDDDWIDWGPTSDDDC

SEQ ID NO: 476 - protein, 16 aa, synthetic construct
  SITE 1: note = ACETYLATION
  SITE 16: note = AMIDATION
  CROSSLNK 1..16: note = two cysteines linked via a non-peptide chemical linker, wherein the non-peptide chemical linker is a o-xylylene linker
CEDDDWIDWYDSDDDC

SEQ ID NO: 477 - protein, 16 aa, synthetic construct
  CROSSLNK 1..16: note = cysteine linked to the N-terminal amino acid residue of the peptide
EDDDWIDWGPTSDDDC

SEQ ID NO: 478 - protein, 15 aa, synthetic construct
  CROSSLNK 1..15: note = cysteine linked to the N-terminal amino acid residue of the peptide
EDDDWIDWYDSDDDC

SEQ ID NO: 479 - protein, 17 aa, synthetic construct
  VARIANT 3: note = ASPARTIC ACID OR THREONINE
  VARIANT 4: note = ASPARTIC ACID OR GLUTAMIC ACID
  VARIANT 5: note = SERINE, ASPARTIC ACID, TYROSINE OR PHENYLALANINE
  VARIANT 6: note = MAY BE DELETED OR TRYPTOPHAN, 3-(2-NAPHTHYL)-L-ALANINE, O-ETHYL-L-TYROSINE, O-BENZYL-L-TYROSINE OR L-CYSTEIC ACID
  VARIANT 7: note = ISOLEUCINE OR THREONINE
  VARIANT 8: note = ASPARTIC ACID, TRYPTOPHAN OR ALANINE
  VARIANT 10: note = TYROSINE OR GLYCINE
  VARIANT 11: note = GLYCINE, PROLINE OR ASPARTIC ACID
  VARIANT 12: note = ALANINE, THREONINE OR SERINE
  VARIANT 13: note = MAY BE DELETED OR GLYCINE, SERINE OR ALANINE
  VARIANT 15: note = ASPARTIC ACID, THREONINE OR GLUTAMIC ACID
  SITE 1: note = ACETYLATION
  SITE 17: note = AMIDATION
  CROSSLNK 1..17: note = two cysteines linked via a non-peptide chemical linker, wherein the non-peptide chemical linker is a o-xylylene linker
CEXXXXXXWXXXXDXDC

SEQ ID NO: 480 - protein, 16 aa, synthetic construct
  VARIANT 2: note = ASPARTIC ACID OR THREONINE
  VARIANT 3: note = ASPARTIC ACID OR GLUTAMIC ACID
  VARIANT 4: note = SERINE, ASPARTIC ACID, TYROSINE OR PHENYLALANINE
  VARIANT 5: note = MAY BE DELETED OR TRYPTOPHAN, 3-(2-NAPHTHYL)-L-ALANINE, O-ETHYL-L-TYROSINE, O-BENZYL-L-TYROSINE OR L-CYSTEIC ACID
  VARIANT 6: note = ISOLEUCINE OR THREONINE
  VARIANT 7: note = ASPARTIC ACID, TRYPTOPHAN OR ALANINE
  VARIANT 9: note = TYROSINE OR GLYCINE
  VARIANT 10: note = GLYCINE, PROLINE OR ASPARTIC ACID
  VARIANT 11: note = ALANINE, THREONINE OR SERINE
  VARIANT 12: note = MAY BE DELETED OR GLYCINE, SERINE OR ALANINE
  VARIANT 14: note = ASPARTIC ACID, THREONINE OR GLUTAMIC ACID
  CROSSLNK 1..16: note = cysteine linked to the N-terminal amino acid residue of the peptide
EXXXXXXWXXXXDXDC